(19) United States
(12) Patent Application Publication
(10) Pub. No.: US 2025/0041096 A1
(43) Pub. Date: Feb. 6, 2025

(54) DEVICES AND METHODS FOR ANCHORING A SLEEVE IN A TISSUE CAVITY

(71) Applicant: AVERTO MEDICAL, INC., Fremont, CA (US)

(72) Inventors: Kenton D. FONG, Belmont, CA (US); Jeffrey W. ETTER, Hayward, CA (US)

(21) Appl. No.: 18/920,711

(22) Filed: Oct. 18, 2024

Related U.S. Application Data
(63) Continuation of application No. PCT/US2023/018931, filed on Apr. 18, 2023.
(60) Provisional application No. 63/332,166, filed on Apr. 18, 2022.

(51) Int. Cl. A61F 5/44; A61B 1/00; A61F 5/445; A61F 5/449; A61M 25/10

(52) U.S. Cl. CPC A61F 5/4408; A61F 5/449; A61M 25/1011; A61B 1/00128; A61F 2005/4455; A61M 2025/1052

(57) ABSTRACT

According to embodiments of the invention, an anchoring system includes a sleeve having an inner surface defining a lumen, a first expandable sealing mechanism disposed along a proximal end of the sleeve, and a second expandable sealing mechanism disposed along the proximal end of the sleeve. The anchoring system further includes open-cell foam disposed on an outer surface of the sleeve. Expansion of the first and second expandable sealing mechanisms and application of negative pressure to the anchoring system, causes a seal to form between the first and second expandable sealing mechanisms, the outer surface of the sleeve, and an inner surface of a tissue cavity. According to some embodiments, a sleeve body is included with the sleeve, with the first expandable sealing mechanism being disposed at a proximal end of the sleeve body and the second expandable sealing mechanism being disposed at a distal end of the sleeve body.

### CROSS-REFERENCE TO RELATED APPLICATIONS

This application claims priority to U.S. Provisional Application No. 63/332,166 filed Apr. 18, 2022, the entire contents of which is hereby incorporated by reference.

### BACKGROUND

### 1. Technical Field

The field of the currently claimed embodiments of this invention relates to medical devices, and, more particularly, to delivery and anchoring medical devices within a tissue cavity and/or a luminal tissue space using a specialized delivery system and endoscope.

### 2. Discussion of Related Art

Delivery and anchoring devices for medical devices exist. But, delivery and anchoring of medical devices within a tissue cavity and/or a luminal tissue space can be challenging. Improvement to existing medical device anchoring is needed, especially, in a tissue cavity and/or luminal tissue space.

### SUMMARY

According to some embodiments of the invention, an anchoring system includes a sleeve having an inner surface defining a lumen, a first expandable sealing mechanism disposed along a proximal end of the sleeve, and a second expandable sealing mechanism disposed along the proximal end of the sleeve. The anchoring system further comprises an open-cell foam disposed on an outer surface of the sleeve. Expansion of the first and second expandable sealing mechanisms and application of negative pressure to the anchoring system, causes a seal to form between the first and second expandable sealing mechanisms, the outer surface of the sleeve, and an inner surface of a tissue cavity.

According to one embodiment, the anchoring system further includes a sleeve body. According to an embodiment, the first expandable sealing mechanism is disposed at a proximal end of the sleeve body and the second expandable sealing mechanism is disposed at a distal end of the sleeve body. According to some embodiments, the sleeve body and sleeve are comprised of the same extruded piece of polymer. According to other embodiments, the sleeve body is a separate tubular structure bonded to the tubular structure of the sleeve. According to some embodiments, the sleeve body of the anchoring system has thicker wall thickness than the sleeve of the system. According to some embodiments, the sleeve body of the anchoring system has a higher durometer than the sleeve of the system. According to some embodiments, the sleeve body of the anchoring system is configured so that the lumen does not collapse closed when the expandable sealing mechanisms are expanded within a tissue cavity and negative pressure is applied to the anchoring system. According to some embodiments, the sleeve body of the anchoring system is configured so that the expandable sealing mechanisms remain in a near perpendicular orientation to the sleeve body when the sealing elements are expanded within a tissue cavity and negative pressure is applied.

According to some embodiments, the distal end of the sleeve body is connected to a proximal end of the sleeve. According to another embodiment, the sleeve body is coextensive with the sleeve. According to some embodiments, the sleeve body is disposed on a proximal end of the sleeve.

According to an embodiment, the sleeve includes a plurality of fluid lumens. According to some embodiments, the plurality of fluid lumens includes one or more of (i) a distal fluid lumen to provide a fluid to the first expandable sealing mechanism, (ii) a proximal fluid lumen to provide a fluid to the second expandable sealing mechanism, (iii) a flushing lumen, (iv) a contrast dye lumen, and (v) a negative pressure lumen.

According to some embodiments of the invention, application of negative pressure creates a frictional force that resists displacement of the sleeve and/or the sleeve body. According to some embodiments of the invention, the application of negative pressure brings the open-cell foam disposed on the outer surface of the sleeve into contact with the inner surface of the tissue cavity thereby creating frictional force that resists displacement of the sleeve.

According to some embodiments of the invention, the first and second expandable sealing mechanisms are expanded by providing a non-compressible fluid (e.g., saline, mineral oil, and/or a dye-based (e.g., iodine-based) contrast solution) to inflate or expand the first and second expandable sealing mechanisms. According to some embodiments of the invention, the first and second expandable sealing mechanisms are expanded by a radiopaque liquid. According to some embodiments of the invention, the first and second expandable sealing mechanisms are expanded by filling with a non-compressible liquid. According to some embodiments the expandable sealing mechanisms are expanded by filling with air. According to an embodiment, once expanded, the first and second expandable sealing mechanisms form a substantially airtight and fluid-tight seal with the inner surface of the tissue cavity. According to some embodiments of the invention, the first and second expandable sealing elements are collapsible.

According to some embodiments of the invention, the sleeve protects the inner surface of the tissue cavity from fecal flow distal to the sleeve body. According to some embodiments, the lumen has a diameter between approximately 1 cm and approximately 6 cm. According to some embodiments, the outer surface of the sleeve and/or the sleeve body has a diameter between approximately 1.1 cm and approximately 6.1 cm. According to some embodiments, the sleeve and/or the sleeve body comprises a flexible material having a Shore A hardness between about 20 A and about 70 A. According to some embodiments, the sleeve and/or the sleeve body has a length that is between about 3 cm and about 25 cm. According to some embodiments, the sleeve and/or the sleeve body has a tubular wall thickness of between about 0.1 mm and about 8 mm. According to some embodiments, the sleeve and/or the sleeve body has a tubular wall thickness that is between about 0.2 mm and about 5 mm.

According to some embodiments of the invention, the open-cell foam comprises a material having an average pore size between about 50 microns and about 1000 microns. According to some embodiments, the open-cell foam comprises a material having an average pore size between about 300 microns and about 600 microns. According to some embodiments, the open-cell foam comprises a material having an average pore size between about 200 microns and about 400 microns. According to some embodiments, the open-cell foam comprises a material having an average pore size between about 100 microns and about 300 microns. According to some embodiments, the open-cell foam is compressible by peristaltic contractions of a patient's bowel. According to some embodiments, the open-cell foam is compressible by negative pressure between the sealing elements, sleeve, and tissue cavity. According to some embodiments, the open-cell foam comprises polyvinyl alcohol, polyurethane foam, or other synthetic polymer. According to some embodiments, the open-cell foam has a tensile strength of at least 50 kpa. According to some embodiments, the open-cell foam has a thickness of between 2 mm and 150 mm. According to some embodiments, the open-cell foam comprises a single tubular piece of foam. According to some embodiments, the open-cell foam comprises multiple pieces of foam. According to some embodiments, the open-cell foam is bonded to the sleeve and/or the sleeve body. According to some embodiments, the open-cell foam has a higher coefficient of friction than that of the sleeve and/or the sleeve body.

According to some embodiments, the first and second expandable sealing mechanisms each comprises an inflatable or expandable elastomeric balloon. According to some embodiments, the first and second expandable sealing mechanisms each comprises multiple expandable elastomeric balloons. According to some embodiments, the first expandable sealing mechanism comprises a single inflatable or expandable elastomeric balloon, while the second expandable sealing mechanism comprises multiple or a plurality of expandable elastomeric balloons. According to an embodiment, the first and second expandable sealing mechanisms have an annular diameter that is greater than an annular diameter of the open-cell foam dispersed around the sleeve and/or the sleeve body.

According to some embodiments of the invention, the sleeve has a column strength of about 3.0 lbs. to 6.0 lbs. According to some embodiments of the invention, the sleeve has a column strength of about 3.0 lbs to 6.0 lbs. According to some embodiments the combined sleeve body and sleeve have a column strength of 3.0 lbs to 6.0 lbs.

According to some embodiments of the invention, the anchoring system further includes a collection bag that is disposed at a distal end of the sleeve and configured to manage enteric contents carried from the anchoring system. According to an embodiment, the anchoring system further includes a retention dressing to attach the anchoring system to skin. According to an embodiment, the anchoring system further includes an extension tubing connected to a distal end of the sleeve, with the extension tubing being configured to extend the anchoring system outside of a patient. According to some embodiments, the extension tubing is between 1 ft and 6 ft. In some embodiments, the extension tubing is connected to an effluence bag.

According to some embodiments of the invention, the anchoring system further includes a scope adapter configured to connect the sleeve to at least one of an endoscope, a sigmoidoscope, or a colonoscope. According to an embodiment, the scope adapter comprises a main body, a compression nut, a compression sleeve, and a compression washer. According to an embodiment, the main body includes a plurality of threads configured to engage with a plurality of grooves provided along an interior surface of the compression nut. According to some embodiments, the anchoring system further includes a connector latch point configured to connect the sleeve of the at least one of an endoscope, a sigmoidoscope, or a colonoscope via the scope adapter. According to some embodiments of the invention, the scope adapter holds an external to body end portion of the anchoring system in a fixed position on the length of the scope. According to some embodiments, the scope adapter is configured to allow for fixing the external to body end portion of the anchoring system to different positions along the length of the scope. In some embodiments, the scope adapter is configured to hold the external to body end portion of the anchoring system with at least 3 lbs of longitudinal pulling or pushing force. In some embodiments, the scope adapter is configured to be quickly released from the external to body end portion of the anchoring system to allow withdrawal of the scope from inside the body leaving the anchoring system in place.

According to some embodiments of the invention, the anchoring system further includes a negative pressure source, wherein negative pressure is applied to the anchoring system by the negative pressure source to maintain one of a constant negative pressure or a variable negative pressure at a level between −50 mmHg and −200 mmHg. According to an embodiment, the anchoring system includes a negative pressure lumen configured to provide negative pressure to the sleeve and/or the sleeve body. According to some embodiments of the invention, the anchoring system further includes an irrigation lumen in fluid connection with the outer surface of the sleeve and/or the sleeve body. According to some embodiments of the invention, the anchoring system further includes an irrigation system in fluid connection with the sleeve and/or the sleeve body, wherein the irrigation system introduces a fluid into the sleeve and/or the sleeve body for irrigation. In some embodiments, the irrigation tube in fluid connection with the outer surface of the sleeve is the same tubing as a pressure tube.

According to some embodiments of the invention, the sleeve has a length that allows it to extend outside the tissue cavity. According to some embodiments, the lumen and first and second expandable sealing mechanisms are compressible by normal peristaltic forces of a patient's bowel. According to some embodiments, a diameter of the first expandable sealing mechanism and the second expandable sealing mechanism is less than or equal to a diameter of the tissue cavity in which the sleeve is to be anchored. According to some embodiments, the anchoring system is configured so that traction on the sleeve can be used to remove the anchoring system from the body cavity. According to some embodiments, the sleeve has a wall thickness that is between about 50 microns and about 5 mm. According to some embodiments, the sleeve has a length that is between about 8 inches and about 72 inches. According to some embodiments, the sleeve has a length that is between about 3 cm and about 25 cm. According to some embodiments, the sleeve has markings along its length that indicate the length of sleeve within the tissue cavity after placement. According to some embodiments, the sleeve body and/or the sleeve is comprised of one or more of silicone, polyurethane, thermoplastic elastomer, rubber, or other polymer. According to an embodiment, the sleeve and the sleeve body are comprised of one continuous tubular extrusion.

According to some embodiments of the invention, a pressure tube is attached to the sleeve along its length. According to some embodiments, a pressure tube is disposed within a wall of the sleeve. According to some embodiments, a pressure tube is integrated into the sleeve and comprises a same material as the sleeve. According to some embodiments, a pressure tube is disposed within an additional lumen along the length of the sleeve.

According to some embodiments of the invention, the sleeve body and/or the sleeve are comprised of one or more of silicone, polyurethane, thermoplastic elastomer, rubber, rubber-like material, or other polymer.

According to some embodiments of the invention, the anchoring system further includes a plurality of pressure tubes in fluid connection with the outer surface of the sleeve and/or the sleeve body.

According to some embodiments of the invention, the anchoring system further includes an effluence bag in fluid connection with the sleeve, the effluence bag configured to receive the content of the sleeve. According to some embodiments, the effluence bag is detachable. In some embodiments, the effluence bag can be emptied of content without detachment from the sleeve.

According to some embodiments of the invention, the sleeve has a proximal end in sealed fluid communication with, and extending distal to, the distal end of the sleeve body and the second expandable sealing mechanism, wherein the proximal end of the sleeve, extending distal to the distal end of the sleeve body, is configured to cover and protect a damaged area of tissue of the tissue cavity from content flowing through the lumen of the sleeve and the sleeve body. According to some embodiments of the invention, the sleeve body is bonded around the sleeve. According to some embodiments the sleeve body and the sleeve of the anchoring system are a single continuous piece of polymer, such that the sleeve body and the sleeve share the same lumen.

According to some embodiments of the invention, the sleeve and/or the sleeve body and the first and second expandable sealing elements are made from a single injection mold using a single material. According to some embodiments, the sleeve comprises a releasable, fluid-tight connector at between 8 inches and 36 inches from the second expandable sealing mechanism. According to some embodiments, the sleeve comprises a separation junction at between 8 inches and 36 inches from the second expandable sealing mechanism. According to some embodiments, the anchoring system is configured to be positioned in the tissue cavity using an endoscope.

According to some embodiments, the tissue cavity is bowel comprising an anastomosis, and wherein the anchoring system is positioned within the bowel such that the anastomosis is located distal in the bowel to the second expandable sealing mechanism. According to some embodiments, the anchoring system further includes an irrigation system in fluid connection with the sleeve and/or the sleeve body, wherein the irrigation system introduces a fluid into the sleeve and/or the sleeve body for irrigation.

According to some embodiments, there is an irrigation tubing that extends from outside the body to the bowel, proximal within the bowel to the first expandable sealing mechanism. In some embodiments, this irrigation tubing is used to dilute stool that impacts the proximal within the bowel end of the anchoring system.

According to some embodiments, there is an additional irrigation tubing that extends from outside the body to the bowel, distal within the bowel to the second expandable sealing mechanism. In some embodiments, this additional irrigation tubing is used to irrigate the anastomosis. In some embodiments, this additional irrigation tubing is used to inject contrast to check for an anastomotic leak with a radiographic study.

According to some embodiments of the invention, a delivery system includes an outer protective sheath that encases the anchoring system according to embodiments of the invention, a handle, a guide shaft, and a sheath pull handle. The anchoring system is configured to be pushed into position by advancing the guide shaft into a patient's bowel. According to some embodiments, the delivery system outer protective sheath is withdrawn through the center of the guide shaft to expose the expandable seals and an air conducting rough surface material (or open cell foam) disposed on the outer surface of the sleeve body. In some embodiments, the outer protective sheath only extends to cover the sleeve body of the anchoring system containing the sealing elements and foam.

According to some embodiments, a delivery system is provided that includes a handle, a guide shaft connected to the handle, an outer protective sheath, and a sheath pull handle attached to the outer protective sheath. According to an embodiment, the outer protective sheath is disposed within a central lumen of the guide shaft and is configured to extend from the central lumen in order to cover and protect a proximal end of an anchoring system having at least one expandable sealing mechanism. According to an embodiment, the delivery system is used with an anchoring system according to embodiments of the invention having a sleeve with a column strength that is high enough to prevent collapse of the system during insertion via the delivery system.

According to some embodiments of the invention, an air conducting rough surface material is disposed on the outer surface of the sleeve body or proximal in the body portion of the sleeve. According to some embodiments, the air conducting rough surface material is a stacked mesh matrix, a honey-comb lattice of interconnected channels, gauze, fabric, or a three-dimensional woven material, which can be, e.g., oriented in a radial fashion around the sleeve.

According to some embodiments of the invention, a method for anchoring a sleeve in a tissue cavity, the sleeve having an outer surface comprising foam for contacting an inner wall of the tissue cavity, and an expandable sealing mechanism for isolating a portion of the tissue cavity adjacent to the sleeve from a remainder of the tissue cavity, includes inserting the sleeve in the tissue cavity. The method further includes inflating or expanding the expandable sealing mechanism to create a seal between the expandable mechanism and the inner surface of a tissue cavity, and applying a negative pressure to a region between an outer surface of the sleeve and an inner surface of the isolated portion of the tissue cavity to create a frictional force between the foam of the sleeve and the inner surface of the tissue cavity. According to one embodiment, the sleeve further comprises a sleeve body.

According to some embodiments of the invention, the step of inflating or expanding the expandable sealing mechanism is conducted by injecting a non-compressible liquid into the expandable sealing mechanism. According to some embodiments of the invention, the step of inflating or expanding the expandable sealing mechanism is conducted by injecting an inflation media (e.g., saline, mineral oil, and/or a dye-based (e.g., iodine-based) contrast solution) into the expandable sealing mechanism. According to some embodiments, the step of inflating or expanding the expandable sealing mechanism is conducted by injecting radiopaque contrast into the expandable sealing mechanism.

According to some embodiments of the invention, the step of inserting the sleeve in the tissue cavity is conducted using a delivery system that includes a handle and a guide shaft. According to an embodiment, the method further includes a step of withdrawing the handle and the guide shaft of the delivery system from the sleeve.

According to some embodiments of the invention, the method further includes removing the sleeve from the tissue cavity by (i) releasing the negative pressure, (ii) collapsing the expandable sealing mechanism, and (iii) injecting an amount of saline through an irrigation tubing to break the seal.

According to some embodiments of the invention, an anchoring device is provided that utilizes two redundant methods of anchoring within the bowel, wherein the first method is to utilize a negative pressure based-friction anchor as described herein and the second method is to fixate the external portion of the device to skin with a column strength to the sleeve that is high enough to hold the anchor portion of the device in place even if there is failure of the first method of anchoring; and such that the first method of fixation is sufficient to hold the anchor portion of the device in place if there is failure of the second method. According to an embodiment of the invention, a method of inserting the anchoring system according to embodiments of the invention is provided that uses an endoscope that is connected to the anchoring system. According to some embodiments, the anchoring system is inserted using direct visualization via the endoscope.

According to some embodiments of the invention, an anchoring system includes a sleeve having an inner surface defining a lumen, a first expandable sealing mechanism disposed along a proximal end of the sleeve, and a second expandable sealing mechanism disposed along the proximal end of the sleeve. The anchoring system further an air conducting rough surface material disposed on the outer surface of the sleeve. Expansion of the first and second expandable sealing mechanisms and application of negative pressure to the anchoring system, causes a seal to form between the first and second expandable sealing mechanisms, the outer surface of the sleeve, and an inner surface of a tissue cavity. According to some embodiments, the system further includes a sleeve body, with the first expandable sealing mechanism disposed at a proximal end of the sleeve body and the second expandable sealing mechanism disposed at a distal end of the sleeve body. According to some embodiments of the invention, the air conducting rough surface material is at least one of a stacked mesh matrix, a honey-comb lattice of interconnected channels, gauze, fabric, or a three-dimensional woven material.

According to some embodiments of the invention, an anchoring device configured to be anchored within a bowel of a patient is provided. The anchoring device comprises a sleeve configured to be positioned within the bowel of the patient, an external portion configured to extend externally from the bowel of the patient, and two redundant methods of anchoring the device within the bowel, wherein a first method of anchoring the device utilizes a negative pressure-based system that applies negative pressure to the device to create a frictional force that resists displacement of the sleeve of the device from the bowel, wherein a second method of anchoring the device fixates the external portion of the device to the skin of the patient, wherein at least one of (i) a column strength of the sleeve is high enough to hold the device in place even if there is failure of the first method of anchoring, or (ii) the first method of anchoring is sufficient to hold the device in place even if there is failure of the second method of anchoring.

According to one embodiment, the anchoring device further includes a third redundant method of anchoring the device, wherein the third method of anchoring utilizes first and second expandable sealing mechanisms, wherein expansion of the first and second expandable sealing mechanisms causes a seal to form between the first and second expandable sealing mechanisms and an inner surface of a tissue cavity, wherein the third method of anchoring is sufficient to hold the device in place even if there is failure of the first and/or the second method of anchoring.

### BRIEF DESCRIPTION OF THE DRAWINGS

Further objectives and advantages will become apparent from a consideration of the description, drawings, and examples.

FIG. 1A is an illustration of an anchoring system according to some embodiments of the invention.

FIG. 1B is an illustration of an end view of an expandable sealing mechanism of an anchoring system according to an embodiment of the invention.

FIG. 1C is a schematic illustration of an end view of an expandable sealing mechanism of an anchoring system according to an embodiment of the invention.

FIG. 1D is a schematic illustration of an end view of an expandable sealing mechanism of an anchoring system according to an embodiment of the invention.

FIG. 2A is a schematic illustration of an anchoring system in a collapsed position according to some embodiments of the invention.

FIG. 2B is a schematic illustration of an end view of the anchoring system shown in FIG. 2A according to an embodiment of the invention.

FIG. 2C is a schematic illustration of an end view of the sleeve lumens of an anchoring system according to some embodiments of the invention.

FIG. 3 is a schematic illustration of an anchoring system according to some embodiments of the invention.

FIG. 4A is a schematic illustration of an anchoring system in a collapsed position according to an embodiment of the invention.

FIG. 4B is a schematic illustration of an anchoring system in an expanded position according to an embodiment of the invention.

FIG. 5 is a schematic illustration of an exploded view of each of the components of an anchoring system according to some embodiments of the invention.

FIG. 6A illustrates a method for insertion of the anchoring system in a tissue cavity with a flexible member and semi-rigid tube pusher.

FIG. 6B illustrates the anchoring system detached from the delivery system (flexible member and pushing member removed) and in the desired position.

FIG. 6C illustrates the anchoring system once negative pressure is applied through the pressure tube with collapse of the bowel wall around the sealing members and anchor sleeve body.

FIG. 7A is a schematic illustration of a distal fluid path of an anchoring system in an expanded position according to an embodiment of the invention.

FIG. 7B is a schematic illustration of a proximal fluid path of an anchoring system in an expanded position according to an embodiment of the invention.

FIG. 8 is a schematic illustration of a contrast lumen of an anchoring system in an expanded position according to an embodiment of the invention.

FIG. 9 is a schematic illustration of a negative pressure path of an anchoring system in an expanded position according to an embodiment of the invention.

FIG. 10A illustrates a delivery system for the anchoring system according to some embodiments of the invention.

FIG. 10B is a schematic illustration of a delivery system in combination with an anchoring system according to an embodiment of the invention.

FIG. 10C is a cross-sectional view of the delivery system in combination with an anchoring system as shown in FIG. 10B according to an embodiment of the invention.

FIG. 10D is a partial, expanded, cross-sectional view of the handle of the delivery system in combination with an anchoring system as shown in FIGS. 10B and 10C according to an embodiment of the invention.

FIG. 10E is a partial, expanded, cross-sectional view of an end of the anchoring system with the delivery system as shown in FIGS. 10B and 10C according to an embodiment of the invention.

FIG. 10F is a cross-sectional view of the anchoring system with the delivery system as shown in FIG. 10E taken along line A-A of FIG. 10E according to an embodiment of the invention.

FIG. 10G is a cross-sectional view of the anchoring system with the delivery system as shown in FIG. 10E taken along line B-B of FIG. 10E according to an embodiment of the invention.

FIG. 10H is a cross-sectional view of the anchoring system with the delivery system as shown in FIG. 10E taken along line C-C of FIG. 10E according to an embodiment of the invention.

FIG. 10I is a cross-sectional view of the anchoring system with the delivery system as shown in FIG. 10E taken along line D-D of FIG. 10E according to an embodiment of the invention.

FIG. 10J is a cross-sectional view of the anchoring system with the delivery system as shown in FIG. 10E taken along line E-E of FIG. 10E according to an embodiment of the invention.

FIG. 11A is a schematic illustration of a delivery system for use with an anchoring system according to another embodiment of the invention.

FIG. 11B is a cross-sectional view of the delivery system for use with an anchoring system as shown in FIG. 11A according to an embodiment of the invention.

FIG. 11C is a partial, expanded, cross-sectional view of an end of the delivery system as shown in FIGS. 11A and 11B according to an embodiment of the invention.

FIG. 11D is a partial, expanded, cross-sectional view of the handle of the delivery system as shown in FIGS. 11A and 11B according to an embodiment of the invention.

FIG. 12 illustrates an extension tubing for the anchoring system according to some embodiments of the invention.

FIG. 13 shows an embodiment of the anchoring system that includes two anchor elements to deliver therapeutic agents to an isolated segment of bowel.

FIG. 14 shows a side view of an embodiment of the anchoring system that includes two anchor elements to deliver therapeutic agents to an isolated segment of bowel.

FIGS. 15A and 15B are schematic illustrations of a scope adapter according to some embodiments of the invention.

FIG. 16 is a cross-sectional view of the scope adapter shown in FIGS. 15A and 15B taken along line 16-16 of FIG. 15A according to an embodiment of the invention.

FIG. 17 is a schematic illustration of an anchoring system attached to an endoscope via a scope adapter according to some embodiments of the invention.

FIG. 18 is a cross-sectional view of the scope adapter shown in FIG. 17 taken along line 18-18 of FIG. 17 according to an embodiment of the invention.

FIG. 19A is a schematic illustration of a retention dressing to secure an anchoring system to a patient according to some embodiments of the invention.

FIGS. 19B-19H are schematic illustrations of a method to attach the retention dressing shown in FIG. 19A to an anchoring system according to an embodiment of the invention.

FIG. 20A is a schematic illustration of a collection bag according to some embodiments of the invention.

FIG. 20B is a schematic illustration of a pair of attachment straps to attach the collection bag of FIG. 20A to a patient according to an embodiment of the invention.

FIG. 21A is a schematic illustration of a collection bag according to some embodiments of the invention.

FIG. 21B is a side view of the collection bag of FIG. 21A according to an embodiment of the invention.

### DETAILED DESCRIPTION

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, terms such as “comprising” “including,” and “having” do not limit the scope of a specific claim to the materials or steps recited by the claim.

As used herein, terms such as “consisting of” and “composed of” limit the scope of a specific claim to the materials and steps recited by the claim.

All prior patents, publications, and test methods referenced herein are incorporated by reference in their entireties.

The need for temporary protection of the bowel lumen and/or a segment of the bowel from enteric fecal flow after surgical bowel resection and anastomosis or when the bowel wall is damaged (e.g., tissue damage) is a common problem in modern medical treatment. Examples include, e.g., protection of newly formed surgical anastomoses, anatomic leaks, inflamed or irritated bowel, partial thickness injuries to the bowel wall, and full thickness bowel perforations. A traditional means of temporarily protecting the bowel from enteric flow is by creating an external diversion of the bowel through the creation of an ostomy or stoma. An ostomy is a purposeful anastomosis between a segment of the gastrointestinal (GI) tract and the skin of the anterior abdominal wall. Basically, a surgeon diverts a segment of bowel proximal to the region to be protected to the skin, bypassing stool flow away from the vulnerable bowel segment. An ostomy can be created virtually anywhere along the GI tract. For diversion of the fecal stream, the most common ostomies involve the distal small intestine (e.g., ileostomy) and large intestine (e.g., colostomy). Ostomies are performed in around 300,000 patients in the US and over 2 million patients globally each year, but this surgery is complicated by high morbidity, mortality, and severe impact on a patient's quality of life. Complications such as, e.g., parastomal hernias, infections/sepsis, skin irritation and erosion, electrolyte depletion, dehydration, and prolapse are common. Up to 50% of patients who undergo ostomy surgery will have some type of ostomy related complication, with many requiring re-hospitalization or additional operations. Although many ostomies are intended to be temporary, as many as ⅓ of temporary ostomies are never reversed, due to, for example, the patient's fear of undergoing further surgery or risk factors associated with complications from the original surgery. In addition, the reversal surgery itself is associated with high complication rates (17%) and significant mortality (1.2-7%). Accordingly, there is a need for improved methods and devices to provide a less morbid alternative for fecal diversion for colonic protection.

One of the major indications for a temporary ostomy is to protect a bowel anastomosis after bowel resection surgery. The protective ostomy or stoma is performed to both protect the newly formed anastomosis from fecal flow and to prevent the development of severe sepsis if an anastomotic leak develops. An anastomotic leak is defined as a defect of the intestinal wall at the anastomotic site leading to a communication between the intra- and extraluminal compartments. The overall incidence of colorectal anastomotic leak varies widely in the literature, ranging from 1 to 24%. Leaks can cause severe complications such as loss of the anastomosis, sepsis, and death. Even in those cases where the anastomosis is salvaged, poor compliance in the neorectum can lead to a poor functional outcome. In many large studies, anastomotic leaks have been shown to be associated with a pelvic sepsis at a rate of 50%. By protecting the anastomosis from fecal flow, anastomotic leaks may be prevented, or perhaps more importantly, their complications of severe sepsis and death mitigated. This is why colon surgeons perform temporary ostomy surgeries after bowel resections even though the vast majority of patients will likely heal uneventfully. Even after an anastomotic leak has occurred, protection from fecal flow can make the anastomotic leak less severe and aid in healing of the leak. There are several risk factors for the development of an anastomotic leak. The most significant risk factor is the level of the anastomosis, with the leak rate increasing as the distance from the anastomosis to the anus decreases. Other than meticulous technique in creating the anastomosis, the major strategy to prevent and treat anastomotic leaks during complicated or high-risk cases involving bowel resection is to divert fecal flow with a protective ostomy. This is accomplished by having the flow of enteric contents diverted using an ostomy created in the bowel proximal to the anastomosis or anastomotic leak. Proximal in the bowel is defined as higher up in the GI tract towards the mouth, distal in the bowel is defined as lower down in the GI tract towards the anus. This ostomy can be either an end ostomy such as an end colostomy or end ileostomy or can be a diverting loop ileostomy that does not completely disrupt bowel continuity.

A temporary diverting ostomy and its closure has its own set of complications and morbidities including dehydration due to high output, difficulty with ostomy care, stricture at the closure site, wound infections, and incisional hernias. Complication rates of ostomies range between 5% and 100%. The complications can be divided into minor complications, which do not require surgical intervention, and major complications requiring surgical intervention. Major complications include stenosis, small bowel obstruction, retraction, necrosis, prolapse, stricture, fistula, and parastomal hernia. In some cases, such as partial small bowel obstruction, the patient can first be treated conservatively and surgical intervention may be avoided. Major complications such as ostomy necrosis that extends more than a few millimeters, surgical intervention is mandatory. Minor complications include dermatitis, electrolyte imbalance, and dehydration from high ostomy output, although the last often necessitates early closure of the ostomy. For major complications, additional costs and morbidity associated with additional operations or hospitalizations can be significant. Even for minor complications, treating complications and providing ostomy education can be burdensome to healthcare providers and patients. Some complications such as hernia, prolapse, and stenosis may become chronic and often require multiple corrective operations and associated costs. Ostomies also significantly reduce a patient's quality of life. Fecal output from the ostomy is collected into an ostomy bag attached to the patient's abdomen. These bags need to be emptied and replaced regularly to properly care for the ostomy and prevent unintentional discharge of fecal material. Ostomy appliances can fail and lead to leakage. The burden of living with an ostomy can negatively impact a patient's social life and restrict physical activities.

Furthermore, the reversal of an ostomy is a surgical procedure fraught with potential complications, as often times the abdominal compartment has dense adhesions that make re-establishment of normal bowel continuity both technically challenging and potentially morbid. The repaired bowel after ostomy takedown may also develop a leak at the repair site or anastomotic site in cases of loop ileostomies or in cases of end ostomy reattachment, respectively. Because of the potential for bowel injury and development of a leak from ostomy reversal surgery, the procedure is also associated with significant mortality. In addition to expenses associated with taking patients to the operating room, patients typically require a hospitalization of 2-4 days post-procedure until bowel function returns. Furthermore, the reversal of an ostomy may be too risky or impossible in some patients, requiring the patient to live the rest of their life with an ostomy.

Besides anastomotic protection, there are other potential indications for temporary fecal diversion. These include: 1) treatment of an anastomotic leak after it has occurred, 2) diverticulitis, 3) inflammatory bowel diseases such as Crohn's or Ulcerative Colitis, 4) intestinal perforation and 5) other less common instances of bowel injury where fecal diversion could be useful such as in cases of ischemic bowel disease, bowel contusion injury from trauma, or non-healing perineal/perianal wounds. When a leak or bowel perforation has occurred, such as, in cases of anastomotic leak and diverticulitis, as examples, treatment with fecal diversion can reduce the severity and extent of the condition. Thus, these patients may heal their leak/perforation faster and not develop more severe complications when continued fecal flow contamination of the affected site is mitigated. Inflammatory conditions of the bowel wall such as Crohn's disease or Ulcerative Colitis can make the intestinal lining susceptible to damage from fecal flow. Continued fecal flow can further inflame and contaminate the bowel wall and lead to worsening of the patient's overall disease, infection/sepsis, or even perforation of the bowel wall. Protection from fecal flow allows the inflamed sections of bowel to heal, and, potentially, fecal diversion could reduce recovery time, hospitalization time, and limit severe complications, such as, e.g., perforations or fistula formation. Patients with these conditions may not be good candidates for surgery due to their concomitant conditions or sepsis; thus, performing major surgery to create an ostomy can be morbid in these cases. Accordingly, there is a need for improved methods and devices to provide a less morbid alternative for temporary fecal diversion.

More recently, intraluminal diversion has been attempted using a protective sleeve/sheath within the bowel as an alternative to temporary ostomy formation. Instead of diverting stool outside the body with an ostomy/stoma, the stool is diverted through the lumen of the bowel inside a protective sleeve/sheath effectively protecting the bowel wall from fecal contamination. However, anchoring of the sleeve/sheath securely and safely within the bowel lumen has proven to be historically challenging. Previous attempts at anchoring within the bowel have all either caused potentially catastrophic complications (erosion or ischemic injury to bowel wall, increased anastomotic leak rates) or unreliable anchoring and protection (early extrusion, device migration, incomplete fecal bypass). Historically, these sleeves have been inserted during a laparoscopy or laparotomy under direct visualization or blindly without visualization. However, avoiding inadvertently causing damage to the bowel during blind insertion of a protective sleeve/sheath can be risky in the setting of bowel wall damage or other bowl pathology such as inflammation that can make the bowel wall more susceptible to iatrogenic injury. However, there are many clinical scenarios were placement of the anchor and attached sleeve is needed without an associated surgical laparoscopy or laparotomy. In these cases, performing a laparoscopy or laparotomy to assist in placement of the device would expose the patient to additional risks and potential complications from surgery that might limit the indications for use to only the most severe cases where the benefits of colonic protection outweighed the potential morbidity of abdominal surgery. The ability to anchor a protective sleeve/sheath safely without performing a laparoscopy or laparotomy would increase the indications for use of the protective device and minimize morbidity from using the device. Thus, there exists a need for a device to protect a segment of bowel that can be safely placed without laparoscopy or laparotomy through a completely intraluminal approach. There are many clinical examples where this technology could be beneficial. For example, conditions that require temporary colon protection could include, e.g., high risk colonoscopy, inflammatory bowel disease, or diverticulitis.

Disclosed herein are systems and methods for anchoring a protective sleeve within the bowel proximal to a region of bowel that requires protection from fecal flow, such as a bowel anastomosis or area of bowel damage. The system and methods can make temporary fecal diversion ostomy surgery unnecessary in most patients, as it provides internal fecal diversion and accomplishes the same overall objective as a temporary ostomy by protecting the distal segment of bowel from fecal flow. In addition, we disclose additional configurations of this system that enable drug delivery to the intestinal lumen.

According to an embodiment, an anchoring system or an Intraluminal Colonic Diversion (ICD) System is provided that is a medical device designed to temporarily protect a segment of colon from fecal flow. The system or product is intended to improve patient care by reducing the need for temporary ostomies after bowel wall damage. The ICD system is a two-part system that includes (i) a sterile, single patient use ICD Device and (ii) a sterile, single patient use ICD Delivery System. The ICD Device and ICD Delivery System are assembled ready-to-use in a pouch, such as, e.g., a Tyvek-PET/LDPE pouch, and the sealed pouch is packaged in a single chipboard box as the complete ICD System.

According to some embodiments, the system (i.e., the ICD System) includes an anchoring mechanism that allows for non-traumatic and reversible anchoring of a sleeve within the GI tract that diverts fecal contents away from the anastomotic site or area of damaged bowel. The device is designed to be left in place for a period of a few days to several weeks (including, e.g., 10 to 14 days, up to 21 days, and/or at least four weeks), and then removed completely from the patient after healing has occurred or diversion is no longer required. While the device and method are described here in the context of securely anchoring a sleeve within the GI tract for the purpose of therapeutic benefit such as diverting bowel contents, the device and method for anchoring may also have applications in other regions of the body where secure anchoring within a tissue cavity is desired. It is important to emphasize that this is a device designed to be substantially and securely anchored in place within the bowel and to prevent substantial device migration until the device is actively disengaged and removed by the clinician. This is in contrast to other non-surgically attached sheath or sleeve-based protection devices that are unable to be securely anchored and are slowly extruded from the bowel over time because they cannot maintain the same high level of anchoring strength required to resist bowel expulsion forces. The unique design of the device disclosed herein allows for it to anchor in place within the bowel without dislodgement, without damaging the bowel wall, without the need for a surgical fixation such as suturing, stapling, or external to the bowel retention rings, and without the need for a permanent implant. Each of these features are described in more detail below.

According to an embodiment, the ICD device includes a specialized negative pressure anchor and an associated protective sleeve designed to reliably, reversibly, and safely protect the distal bowel (i.e., the bowel downstream in the gastrointestinal (GI) tract from the anchor) from fecal enteric flow. The specialized negative pressure anchor utilizes a unique foam interface (or an air conducting rough surface material) to create an atraumatic, but strong bond to the bowel wall that holds the ICD device in place and prevents migration. According to an embodiment, the anchor portion of the ICD device has expandable sealing mechanisms in the form of, e.g., two soft, inflatable elastomeric balloons that provide atraumatic sealing surfaces to both facilitate negative pressure anchoring and provide a fluid/air tight seal against fecal contamination distal within the bowel to the anchor portion. These seals are also collapsible to allow for withdrawal of the anchor system without straining the bowel wall. Negative pressure is applied to the sealed area between the expandable or expanded sealing mechanisms (e.g., the inflated balloons) and the bowel lumen to activate anchoring. A biocompatible elastomeric open-cell foam material is spaced between the expandable sealing mechanisms (e.g., balloons) to provide a large contact surface and a means for negative pressure to distribute evenly around the outer surface of the anchor sleeve and/or the anchor sleeve body and inner bowel wall. This interface also creates a unique friction bond to the bowel wall that is stronger than what has been previously achieved without more invasive fixation techniques. The ICD device is designed to be placed in the operating/procedure room and can be removed at the patient's bedside or in clinic. The ICD Device does not require surgical fixation, such as, e.g., stapling, extraluminal restriction rings, or expandable wire-stenting, that can damage the delicate bowel wall. The ICD Device can be left in place for typically a 10 to 14-day period, though it may be left up to 21 days or more if necessary, to provide extended protection to a surgical bowel anastomosis or segment of damaged bowel, such as, e.g., an anastomotic leak and/or inflamed bowel.

According to some embodiments, the device includes a negative pressure based anchor that prevents a sleeve and/or a sleeve body from becoming dislodged from the inner surface of the bowel. According to some embodiments, the sleeve includes a sleeve body and/or is connected to a sleeve body and acts in combination as a protective barrier between the GI tract and the GI contents flowing through the sleeve and sleeve body. In some embodiments, the sleeve body is added onto a proximal end of the sleeve. In some embodiments, the sleeve and sleeve body are manufactured as one continuous tubular structure. In some embodiments, the sleeve and sleeve body are separate tubular components that are bonded together. According to one embodiment, the sleeve body is a reinforcing member designed to support the proximal end of the sleeve and/or device and, more specifically, the anchor element. The sleeve body is a key component to functionality of the device and (1) provides added support to allow for expansion of the expandable sealing members (e.g., balloons) without collapse of the sleeve inner lumen, (2) helps maintain the expandable sealing members (e.g., balloons) at an angle perpendicular to the sleeve and provides support to prevent them from twisting sideways as they expand, and (3) maintains an open and patent central lumen of the sleeve at the proximal end by providing necessary added support/strength to the sleeve. This allows for a thinner, softer and more flexible sleeve conduit along the length of the device.

According to some embodiments, the device includes a pneumatic system for applying negative pressure to the anchor system. The device in some embodiments includes an external effluence bag to collect GI content that flows through the sleeve. However, an external effluence bag is not required for the device to function. In some embodiments, the device has a sleeve that is open just external to the anal sphincter and feces can be passed through this opening. In this embodiment, the anal sphincter constricts around the sleeve and provides some continence and a collection bag is not required.

According to some embodiments, the anchoring portion of the device is positioned in the GI tract on the proximal side of an anastomosis or proximal to the area of damaged bowel. The anchoring portion of the device is defined as the portion containing the expandable sealing elements and foam interface. The proximal side is the side that is “upstream” in terms of the flow of GI content through the GI tract. This is in contrast to anastomosis or wound treatment systems that are configured to be applied directly to an anastomosis or wound site. This device is configured to be anchored in healthy undamaged bowel. Constant and/or variable negative pressure is maintained via a pneumatic interface connected to the anchoring system and dispersed through an open cell reticulated foam interface (or an air conducting rough surface materials). Expandable sealing elements at the end of the sleeve and/or the sleeve body create a negative pressure space between the outer surface of the sleeve and/or the sleeve body containing the foam interface and the bowel wall. When negative pressure is applied, the pressure gradient acts through the foam to create adhesive and friction forces between the GI tract and the anchoring system. These adhesive and friction forces created by the negative pressure-sponge interface enable the anchoring system to maintain a relatively fixed position in the bowel that is much greater than other non-surgically fixated sleeve anchoring systems previously described. This fixation is also substantially safer with respect to bowel damage compared to other forms of fixation such as stapling, suturing, expandable stenting, or external fixation rings. When a user is ready to remove the device, normal atmospheric pressure between the anchoring device and bowel can be reestablished, allowing the device to move through the GI tract with minimal friction. The device and method of fixation do not require suturing, stapling, biodegradable implants, external fixation rings, or other invasive anchoring techniques, and create minimal trauma to the bowel. Thus, disclosed herein are a method and device for securely fixing a sleeve and/or a sleeve body within the bowel lumen in a manner that does not substantially damage the bowel wall, and that allows fixation to be easily reversed for device removal.

In accordance with the features of the embodiments of the invention, the device for anchoring the sleeve and/or the sleeve body within the bowel can be described as having a hollow body with one or more expandable sealing elements on one or both ends and a porous material on the external surface of the hollow body, such that upon application of negative pressure to the external surface of the hollow body and expansion of the one or more expandable sealing elements, an adhesive force forms between the bowel wall and hollow body. At least one lumen can deliver negative pressure (e.g., vacuum) to the sealing member, while one or more other lumens can deliver a fluid (e.g., saline, mineral oil, and/or a dye-based (e.g., iodine-based) contrast solution) to inflate the one or more expandable sealing elements. A protective sleeve can be attached to the sealing member and a collection system can collect contents which pass through the sealing member.

According to some embodiments of the invention, an ICD delivery device is provided that includes a handle, a guide shaft, an outer protective sheath, and a sheath pull handle, which allows a physician or surgeon to place the ICD device at the time of surgery, e.g., colon resection surgery. The ICD delivery system facilitates placement of the ICD device by maintaining a small anchor profile during insertion and protecting the expandable sealing mechanisms (e.g., the inflatable elastomeric balloons) from potential damage when inserting the device past a stapled anastomosis. According to an embodiment, the ICD device is inserted, advanced, and positioned with the ICD delivery system transanally. An aqueous-based lubricant such as, e.g., KY-Jelly, is used to facilitate placement. The anchor portion of the ICD device is positioned 5 cm to 10 cm above (more proximal in the GI tract) the area requiring protection from fecal flow (such as, e.g., the anastamosis after rectal cancer surgery). Importantly, the anchor portion of the ICD device is designed to be placed in a healthy bowel and not at the region of damaged bowel where protection is required. After positioning of the anchoring portion, the outer sheath is removed by withdrawing it using a sheath pull handle. In some embodiments, the withdrawal of the outer sheath of the delivery system occurs through the center of the guide shaft. The device is then ready for deployment after the outer sheath of the delivery system is removed.

According to an embodiment of the invention, the ICD device deployment procedure is accomplished by (i) inflating the distal and proximal expandable sealing mechanisms (e.g., the balloons) by injecting a non-compressible fluid (e.g., normal saline, mineral oil, or dye-based or radiopaque contrast) through a syringe connected to distal and proximal filling ports of the ICD device, respectively, (ii) connecting a negative pressure source (e.g., a vacuum source) to a vacuum port to anchor the ICD device and/or activate negative pressure, and (iii) withdrawing the ICD delivery system handle and guide shaft from the ICD device. According to another embodiment, the external portion of the ICD device is further anchored to a patient's skin using an adhesive dressing. In some embodiments, an optional extension tubing can extend the length of tubing to the effluence bag for bedside to gravity use. The ICD device is designed to be easily removed at bedside or in an outpatient clinic without the need for an additional invasive procedure. According to an embodiment, removal of the ICD device is accomplished by (i) disconnecting the negative pressure source (e.g., a vacuum source) and releasing the vacuum, (ii) deflating the distal and proximal expandable sealing mechanisms (e.g., the balloons) by completely removing fluid through a syringe connected to proximal and distal ports of the ICD device, respectively, (iii) injecting a small amount of normal saline through the vacuum port to break the anchoring seal, and (iv) removing the external dressing and gently pulling the ICD device sleeve until the ICD device is fully removed from the anal verge.

According to some embodiments, the ICD device contains at least two additional features. According to one embodiment, an irrigation lumen is provided that is used (if necessary) to instill normal saline proximal to the anchor portion of the device to liquefy formed stool and maintain patency of the diversion sleeve and/or sleeve body. In some embodiments, this irrigation lumen is in communication with the proximal in bowel opening of the sleeve and/or sleeve body. According to another embodiment, a contrast lumen is provided that can be used to inject radiopaque dye distal to the anchor portion of the device within the bowel to perform a radiographic leak test.

According to an embodiment, the ICD system is used with a specialized adhesive dressing, a negative pressure source, and a collection bag. These features may be accessory products to the main system. In addition, according to one embodiment, the ICD system includes a scope adapter that allows for placement of the ICD device within the intestine through the use of an endoscope or colonoscope for direct visualization for guided insertion.

In accordance with the features of the embodiments of the invention, when the ICD device is loaded on the delivery system or on an endoscope, sigmoidoscope, or colonoscope, the ability of the ICD device anchor section to move forward against an external resistance is due to the combined effect of (i) the column strength for the size, shape, and material type of the outer sleeve body and/or the sleeve of the anchoring device, and (ii) the internal support (e.g., guide shaft of the delivery system) provided by the size, shape, and material type for the internal supporting member. The internal support member might include the shaft of a colonoscope or other endoscope or the guide shaft of the ICD delivery system. The unique characteristic of the sleeve and/or the sleeve body allows for a column strength that supports the anchoring system during advancement into the bowel, which allows for the delivery system to function. Moreover, the column strength of the sleeve body and/or the sleeve allows for the external fixation of the ICD device in place. According to one embodiment, the relevant yield strength for column buckling is the compressive yield strength of the chosen material. The column strength of the outer sleeve can be calculated and tailored to the application by proper selection of the material type and material properties. In addition to the column strength of the outer sleeve, the internal support (e.g., guide shaft of the delivery system) prevents collapse and bucking both axially and laterally. Moreover, the added support allows for the use of a softer and/or thinner wall material, while providing the strength needed to overcome the forward resistance needed to insert the device.

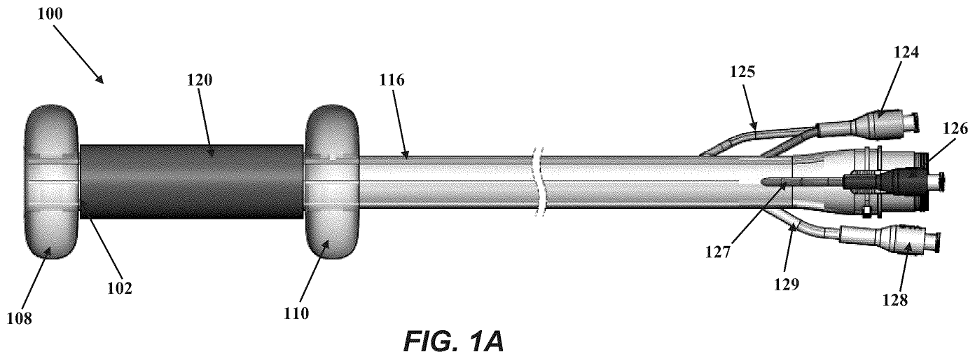

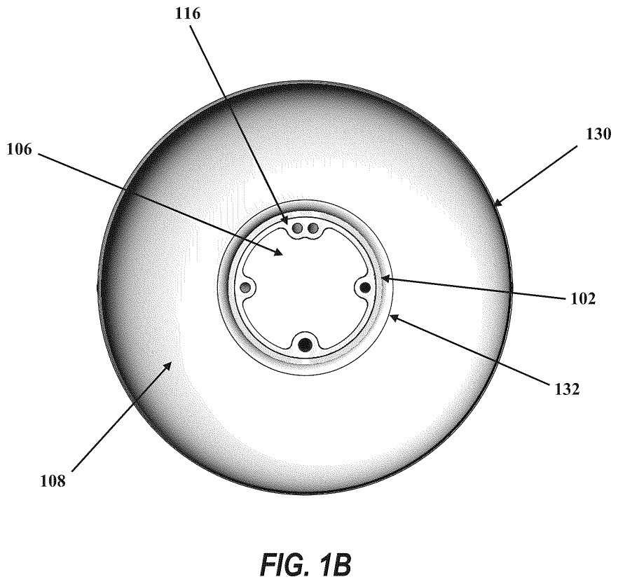

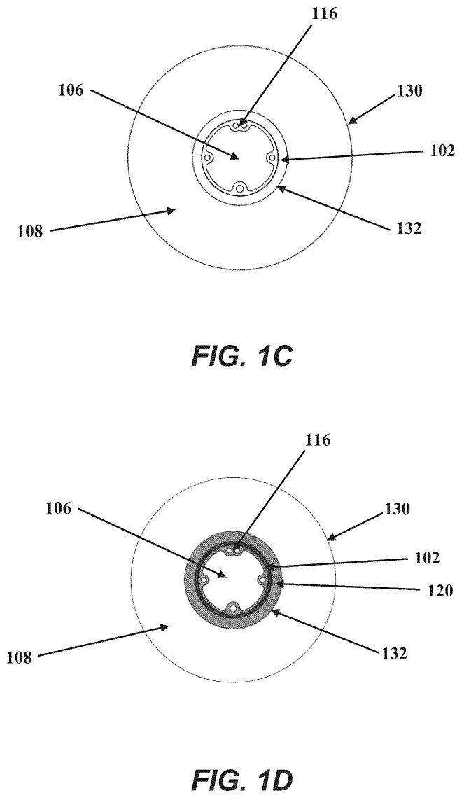

FIG. 1A shows an illustration of an anchoring system of the ICD device according to some embodiments of the invention. The anchoring system 100 includes a sleeve body 102 having an inner surface (not shown) defining a lumen (not shown). A first expandable sealing mechanism 108 is disposed at a proximal end of the sleeve body 102, and a second expandable sealing mechanism 110 is disposed at a distal end of the sleeve body 102. For the device, proximal is defined as the part of the device farthest from where fecal matter exits the sleeve body and/or the sleeve (as defined further below) (at an effluence bag, for example), and distal is defined as the part of the device that is closer to where fecal matter exits the sleeve body and/or the sleeve during regular fecal flow. This orientation convention is used because this is the relationship of flow through the device (from proximal to distal) and matches the orientation of the device within the bowel. A sleeve 116 is in fluid communication with the sleeve body 102, and forms a second lumen (not shown) that is in continuity with the first lumen. Open-cell foam 120 is disposed on the outer surface of the sleeve body 102. The application of negative pressure to the sleeve 116 and/or the sleeve body 102, as well as inflation of the first and second expandable sealing mechanisms 108, 110, causes a seal to form between the first and second expandable sealing mechanisms 108, 110 and an inner surface of the tissue cavity, and creates a frictional force that resists displacement of the sleeve body 102. In some embodiments, the sleeve 116 extends through the sleeve body 102, such that the sleeve 116 is coextensive with the sleeve body 102 (see, e.g., FIGS. 1C and 1D). In another embodiment, the sleeve body 102 is absent and the expandable sealing mechanisms 108, 110 and the foam 120 are attached directly to a proximal end of the sleeve 116. In other embodiments, the distal end of the sleeve body 102 is connected to the proximal end of the sleeve 116.

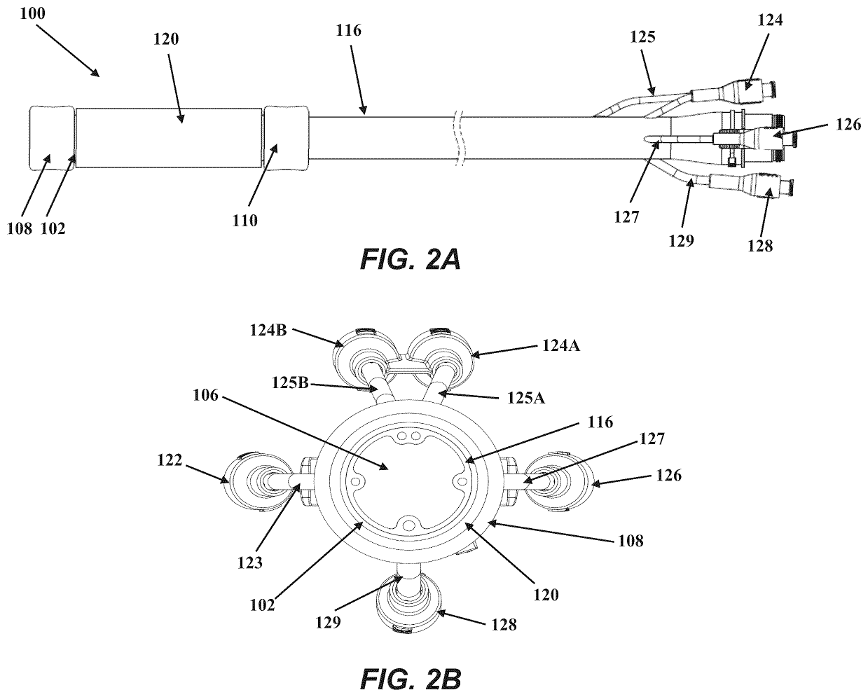

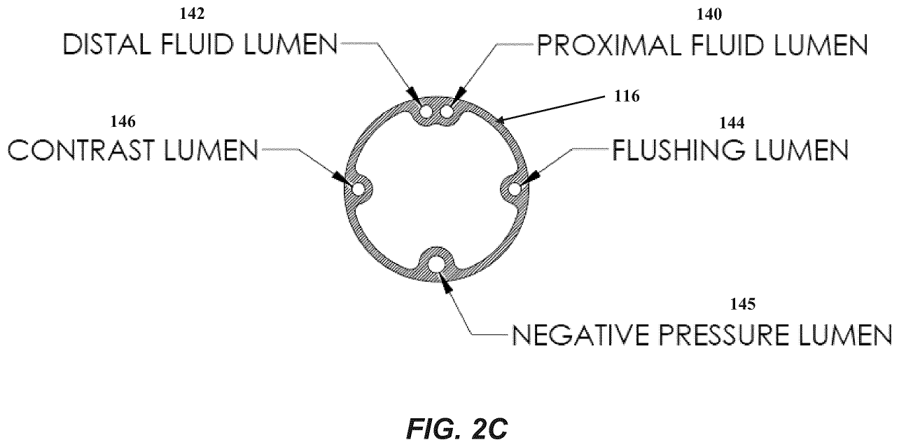

Also shown in FIG. 1A are a series of flexible (or fluid) tubes 125, 127, and 129 and their associated connectors or ports 124, 126, 128, respectively. According to an embodiment, one of the flexible tubes 125 provides a fluid or inflation media (e.g., saline, mineral oil, and/or a dye-based (e.g., iodine-based) contrast solution), e.g., through its associated connector or port 124, to each of the first and second expandable sealing mechanisms 108, 110, in order to inflate the first and second expandable sealing mechanisms 108, 110 with the fluid or inflation media and create a seal between the first and second expandable sealing mechanisms 108, 110 and an inner surface of the tissue cavity. Alternatively, the system 100 may include two flexible tubes 125A and 125B (see, e.g., FIG. 2B), one for each of the first expandable sealing mechanism 108 and the second expandable sealing mechanism 110 to provide a fluid or inflation media (e.g., saline, mineral oil, and/or contrast dye) for inflation. According to one embodiment, a flexible tube or tubing 127, which is in communication with a connector or port 126, can be used to introduce fluid from a fluid infiltration source such, e.g., a syringe. This port 126 can be accessed from outside the patient's body via the flexible tubing 127. This configuration of the anchoring system 100 allows for delivery and removal of irrigation, drugs (such as antibiotics, anti-inflammatory drugs, or chemotherapy agents), and radiologic contrast between the external surface of the sleeve 116 and bowel wall and between the two expandable sealing mechanisms 108, 110. For example, according to one embodiment, the flexible tube 127 and port 126 are provided that can be used to inject contrast dye or radiopaque dye distal to the anchor portion of the device within the bowel to perform a radiographic leak test. According to an embodiment, another flexible tube or tubing 129, such as, e.g., an irrigation tubing, is provided that is used (if necessary) to instill fluid, such as, e.g., normal saline, proximal to the anchor portion of the device to liquefy formed stool and maintain patency of the sleeve body 102 and/or the sleeve 116. For example, according to one embodiment, a fluid, such as, e.g., saline, can be introduced via the flexible tube or tubing 129, which is in communication with a connector or port 128, from a fluid infiltration source such, e.g., a syringe, between the external surface of the sleeve 116 and bowel wall between the two expandable sealing mechanisms 108, 110. Alternatively, the flexible tube or tubing 129 can be used to introduce a negative pressure source (i.e., vacuum) to the sleeve 116 and/or the sleeve body 102 in order to cause a seal to form between the first and second expandable sealing mechanisms 108, 110 and an inner surface of the tissue cavity.

FIG. 1B is an end view of one of the expandable sealing mechanisms according to an embodiment of the invention. As shown in FIG. 1B, the first expandable sealing mechanism 108 includes an external surface 130, an internal surface 132, and an opening that coincides with the lumen 106 of the sleeve body 102 and the sleeve 116. Although not shown in FIG. 1B, the second expandable sealing mechanism 110 also includes the same configuration. As the first expandable sealing mechanism 108 (as well as the second expandable sealing mechanism 110) is inflated using a fluid or inflation media, e.g., saline, mineral oil, and/or a dye-based (e.g., iodine-based) contrast solution, in order to create a seal between the first expanded sealing mechanism 108 (as well as the second expanded sealing mechanism 110) and an inner surface of the tissue cavity, the external surface 130 will become further spaced from the internal surface 132.

FIG. 1C is another end view of one of the expandable sealing mechanisms according to an embodiment of the invention. As shown in FIG. 1C, the first expandable sealing mechanism 108 includes the external surface 130, the internal surface 132, and the opening that coincides with the lumen 106 of the sleeve body 102 and the sleeve 116. As further shown in FIG. 1C, the sleeve body 102 extends radially around the internal surface 132 of the first expandable sealing mechanism 108, and the sleeve 116 extends radially around an inner surface of the sleeve body 102. The sleeve 116 further contains the various fluid lumens (as discussed in further detail below). Although not shown in FIG. 1C, the second expandable sealing mechanism 110 also includes the same configuration.

FIG. 1D is another end view of one of the expandable sealing mechanisms according to an embodiment of the invention. As shown in FIG. 1D, the first expandable sealing mechanism 108 includes the external surface 130, the internal surface 132, and the opening that coincides with the lumen 106 of the sleeve body 102 and the sleeve 116. As further shown in FIG. 1D, the sleeve body 102 and the sleeve 116 are provided within the internal surface 132 of the first expandable sealing mechanism 108. The sleeve 116 contains the various fluid lumens (as discussed in further detail below). In addition, in this end view of FIG. 1D, the foam 120 is illustrated, with the foam 120 being disposed between the internal surface 132 of the first expandable sealing mechanism 108 and the sleeve body 102. Although not shown in FIG. 1D, the second expandable sealing mechanism 110 also includes the same configuration.

FIG. 2A shows another illustration of the anchoring system 100 of FIG. 1A according to an embodiment of the invention. As shown in FIG. 2A, the anchoring system 100 includes the sleeve body 102. The first expandable sealing mechanism 108 is disposed at a proximal end of the sleeve body 102, and the second expandable sealing mechanism 110 is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen 106 (see, e.g., FIGS. 1B-1D, 2B, and 3). The open-cell foam 120 is disposed on the outer surface of the sleeve body 102. As discussed above, the application of negative pressure to the anchoring system 100, as well as inflation of the first and second expandable sealing mechanisms 108, 110, causes a seal to form between the first and second expandable sealing mechanisms 108, 110 and an inner surface of the tissue cavity, and creates a frictional force that resists displacement of the sleeve body 102. Also shown in FIG. 2A are the series of flexible (or fluid) tubes 125, 127, and 129 and their associated connectors or ports 124, 126, 128, respectively, that are provided to introduce a fluid or inflation media, such as, e.g., saline, mineral oil, contrast dye, etc., to the sleeve 116 and/or the sleeve body 102 via the flexible tube or tubing 125, 127, 129, which is in communication with a respective connector or port 124, 126, 128.

FIG. 2B illustrates an end view of the anchoring system 100 illustrated in FIG. 2A at the proximal end. As shown in FIG. 2B, the anchoring system includes the first expandable sealing mechanism 108 disposed at the proximal end of the sleeve body 102 (or the sleeve 116), the sleeve body 102, the lumen 106, and the sleeve 116 that contains the various fluid lumens (as discussed further below). FIG. 2B further illustrates a series of flexible (or fluid) tubes 123, 125A, 125B, 127, and 129 and their associated connectors or ports 122, 124A, 124B, 126, and 128, respectively. According to this embodiment, two flexible tubes 125A and 125B are provided, one for each of the first expandable sealing mechanism 108 and the second expandable sealing mechanism 110 to provide a fluid or inflation media (e.g., saline, mineral oil, and/or a dye-based (e.g., iodine-based) contrast solution) for inflation through its associated connector or port 124A, 124B. According to an embodiment, a flexible tube or tubing 127, such as, e.g., an irrigation tubing, is provided that is used (if necessary) to instill fluid, such as, e.g., normal saline, proximal to the anchor portion of the device to liquefy formed stool and maintain patency of the sleeve body 102 and/or the sleeve 116. For example, according to one embodiment, a fluid, such as, e.g., saline, can be introduced via the flexible tube or tubing 127, which is in communication with a connector or port 126, from a fluid infiltration source such, e.g., a syringe, between the external surface of the sleeve 116 and bowel wall between the two expandable sealing mechanisms 108, 110. According to one embodiment, a flexible tube or tubing 123, which is in communication with a connector or port 122, can be used to introduce fluid from a fluid infiltration source such, e.g., a syringe. This port 122 can be accessed from outside the patient's body via the flexible tubing 123. According to one embodiment, the flexible tube 123 and port 122 are provided to inject contrast dye or radiopaque dye distal to the anchor portion of the device within the bowel to perform a radiographic leak test. According to one embodiment, another flexible tube 129 is provided to deliver a negative pressure source (or vacuum source), via its associated port 128, to the sleeve body 102 and/or the sleeve 116. As discussed above, the application of negative pressure to the sleeve body 102 and/or the sleeve, as well as inflation of the first and second expandable sealing mechanisms 108, 110, causes a seal to form between the first and second expandable sealing mechanisms 108, 110 and an inner surface of the tissue cavity, and creates a frictional force that resists displacement of the sleeve body 102.

FIG. 2C illustrates an end view of the sleeve 116 that includes the various sleeve lumens according to an embodiment of the invention. As shown in FIG. 2C, the sleeve 116 includes (i) a proximal fluid lumen 140 for providing a fluid or inflation media, such as, e.g., saline, mineral oil, and/or a dye-based (e.g., iodine-based) contrast solution, to the first expandable sealing mechanism 108 for inflation of this sealing mechanism, (ii) a distal fluid lumen 142 for providing a fluid or inflation media, such as, e.g., saline, mineral oil, and/or a dye-based (e.g., iodine-based) contrast solution, to the second expandable sealing mechanism 110 for inflation of this sealing mechanism, (iii) a flushing lumen 144 for providing a fluid, such as, e.g., normal saline, to flush or liquefy stool that collects within the lumen 106 of the sleeve 116 and/or the sleeve body 102, (iv) a negative pressure lumen 145 for providing a negative pressure source to the anchoring system, and (v) a contrast lumen 146 for providing a contrast dye to the anchor portion of the device. According to one embodiment, (i) the proximal fluid lumen 140 is in fluid communication with the flexible tube 125A and its associated port 124A, (ii) the distal fluid lumen 142 is in fluid communication with the flexible tube 125B and its associated port 124B, (iii) the flushing lumen 144 is in fluid communication with the flexible tube 127 and its associated port 126, (iv) the negative pressure lumen 145 is in fluid communication with the flexible tube 129 and its associated port 128, and (v) the contrast lumen 146 is in fluid communication with the flexible tube 123 and its associated port 122. According to one embodiment, the fluid lumens (140, 142, 144, 145, 146) run the length of the sleeve 116 and are sealed at each end depending on their function. For example, according to one embodiment, the proximal and distal fluid lumens (140, 142), the contrast lumen (146), the negative pressure lumen (145), and the flushing lumen (144) are sealed at the distal ends by means of a sealed end on a Y-fitting(s). According to an embodiment, the proximal and distal fluid lumens (140, 142), the contrast lumen (146), and the negative pressure lumen (145) are sealed at the proximal ends by a silicone adhesive potted in the end of each lumen. The flushing lumen (144) is generally not sealed at the proximal end in order to allow fluid to exit.

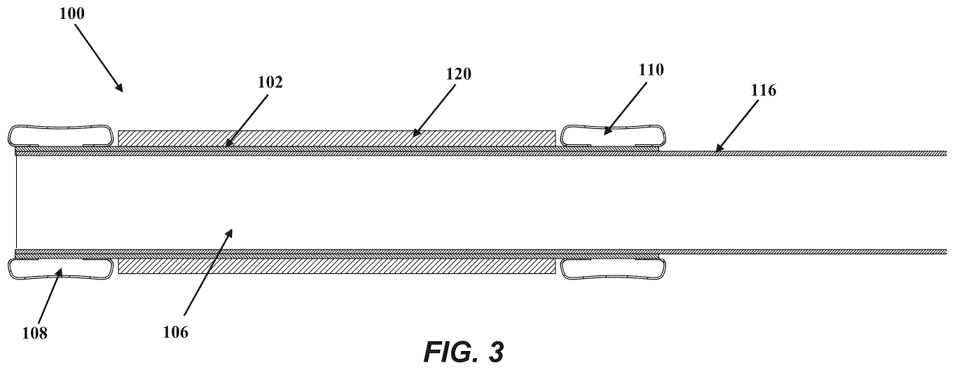

FIG. 3 shows another illustration of the anchoring system 100 of FIG. 1A according to an embodiment of the invention. As shown in FIG. 3, the anchoring system 100 includes the sleeve body 102. The first expandable sealing mechanism 108 is disposed at a proximal end of the sleeve body 102, and the second expandable sealing mechanism 110 is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen 106. The open-cell foam 120 is disposed on the outer surface of the sleeve body 102. As discussed above, the application of negative pressure to the anchoring system 100, as well as inflation of the first and second expandable sealing mechanisms 108, 110, causes a seal to form between the first and second expandable sealing mechanisms 108, 110 and an inner surface of the tissue cavity, and creates a frictional force that resists displacement of the sleeve body 102.

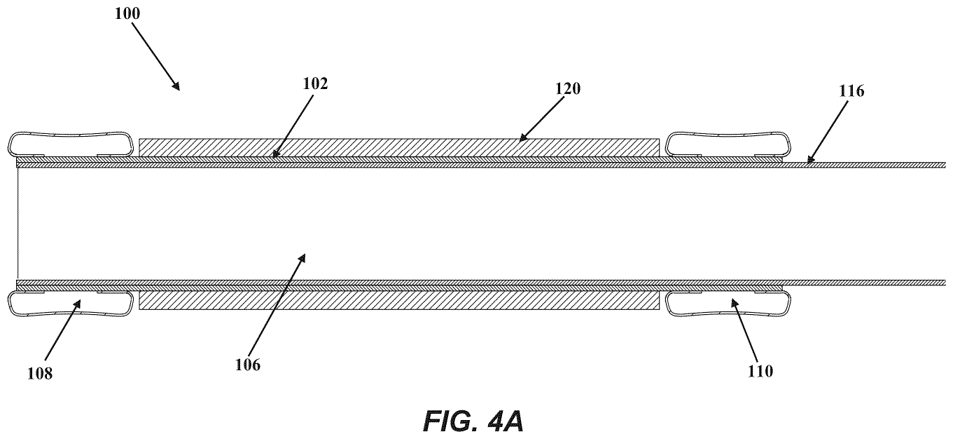

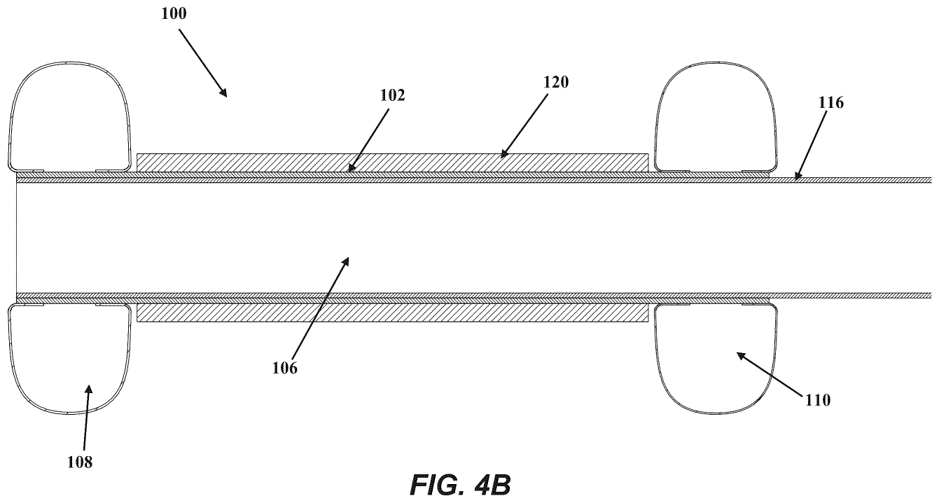

FIG. 4A shows an illustration of the anchoring system 100 of FIG. 1A in which the expandable sealing mechanisms are in a collapsed position, according to an embodiment of the invention. As shown in FIG. 4A, the anchoring system 100 includes the sleeve body 102. The first expandable sealing mechanism 108, which is in a collapsed position, is disposed at a proximal end of the sleeve body 102, and the second expandable sealing mechanism 110, which is also in a collapsed position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen 106. The open-cell foam 120 is disposed on the outer surface of the sleeve body 102.

FIG. 4B shows an illustration of the anchoring system 100 of FIGS. 1A and 4A in which the expandable sealing mechanisms are in an expanded position, according to an embodiment of the invention. As shown in FIG. 4B, the anchoring system 100 includes the sleeve body 102. The first expandable sealing mechanism 108, which is in an expanded position, is disposed at a proximal end of the sleeve body 102, and the second expandable sealing mechanism 110, which is also in an expanded position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with a lumen 106. The open-cell foam 120 is disposed on the outer surface of the sleeve body 102. In the embodiment of FIG. 4B, the first and second expandable sealing mechanisms 108, 110 are expanded by filling the sealing mechanisms with a fluid or inflation media, such as, e.g., saline, mineral oil, and/or a dye-based (e.g., iodine-based) contrast solution, which is delivered via the proximal and distal fluid lumens, respectively (see, e.g., proximal fluid lumen 140 and distal fluid lumen 142 of FIG. 2C) (as discussed in further detail below).

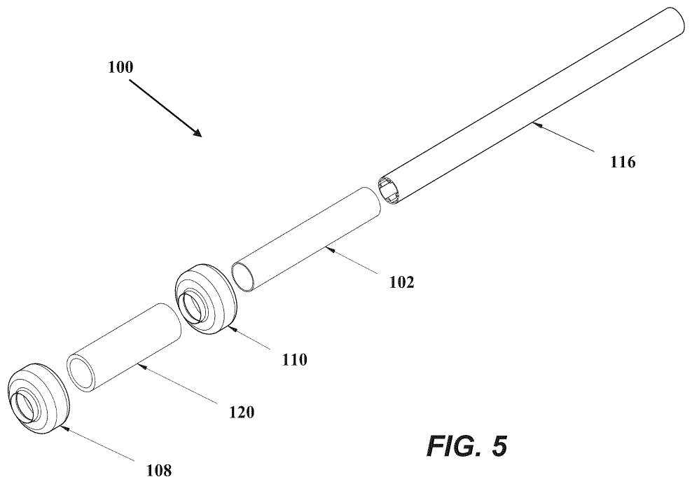

FIG. 5 illustrates an exploded view of each of the components of the anchoring system 100 of FIG. 1A according to an embodiment of the invention. As shown in FIG. 5, the anchoring system 100 includes the sleeve body 102, which either (i) attaches, via the distal end of the sleeve body 102, to the proximal end of the sleeve 116, or (ii) is disposed on top of the proximal end of the sleeve 116. As also shown in FIG. 5, the anchoring system 100 further includes the first (or proximal) expandable sealing mechanism 108 (e.g., balloon) and the second (or distal) expandable sealing mechanism 110 (e.g., balloon), as well as the foam 120. According to an embodiment, the first expandable sealing mechanism 108 is disposed at the proximal end of the sleeve body 102, and the second expandable sealing mechanism 110 is disposed at the distal end of the sleeve body 102. The open-cell foam 120 is disposed on the outer surface of the sleeve body 102. In some embodiments, as discussed above, the sleeve body 102 is absent and the expandable sealing mechanisms 108, 110 and the foam 120 are attached directly to a proximal end of the sleeve 116.

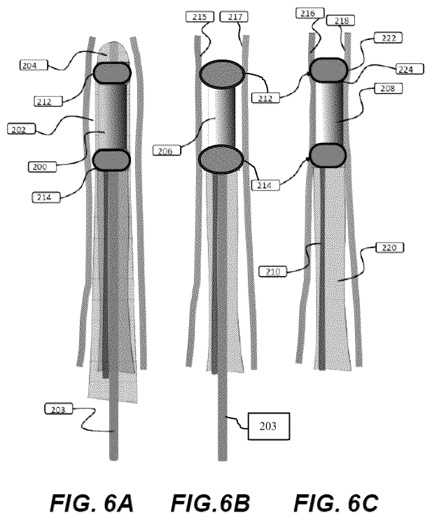

FIGS. 6A-6C illustrate a method for insertion and anchoring of an anchoring system 200. The anchor portion of the anchoring system 200 comprising the sleeve 220, expandable sealing mechanisms 212, 214, and foam dispersed around the sleeve body is transmitted through the tissue cavity 202 to the anchoring site. In the case of an anastomosis, the device is delivered to a position proximal to the anastomosis, such that the sleeve and sealing mechanisms are all proximal to the anastomosis. A semi-rigid tube pusher 203 is used to position the device in the appropriate position. A flexible membrane or protective sheath 204 covers the device and provides coverage and protection of the expandable sealing mechanisms 212, 214 to reduce friction during placement and prevent damage to the sealing mechanisms. The flexible membrane 204 also further reduces friction by covering the foam dispersed over the sleeve body. The device may also be delivered using an endoscope or other delivery system without the use of the flexible member cover. Example delivery systems are discussed in detail below.

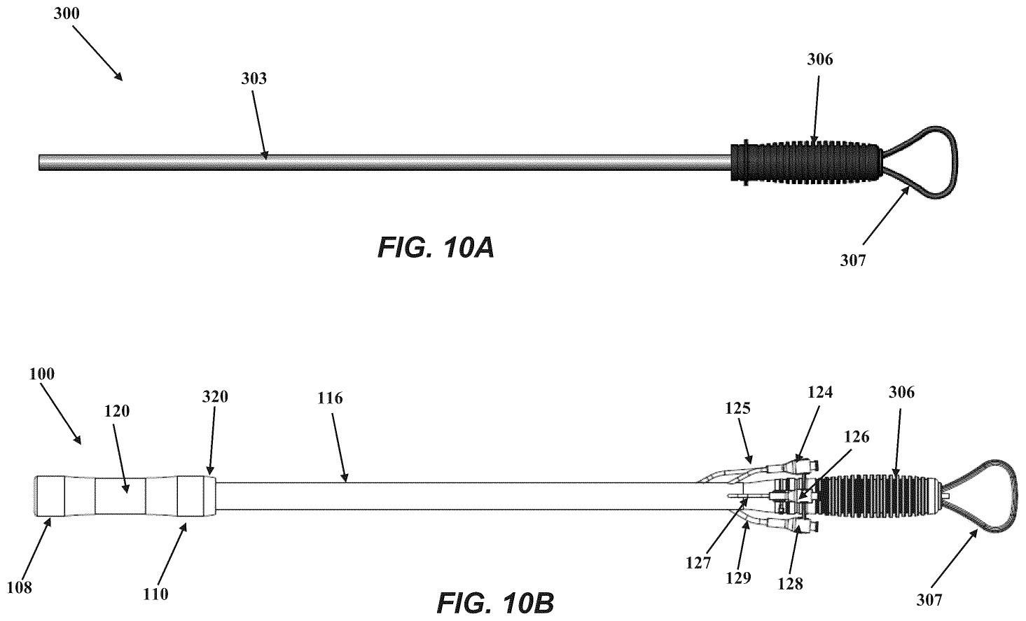

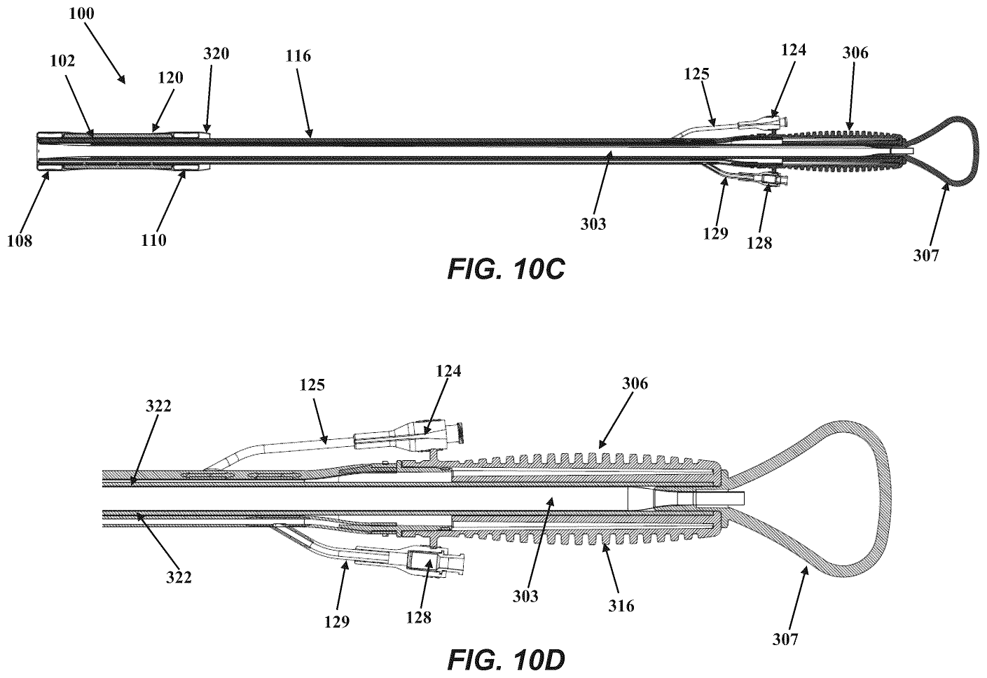

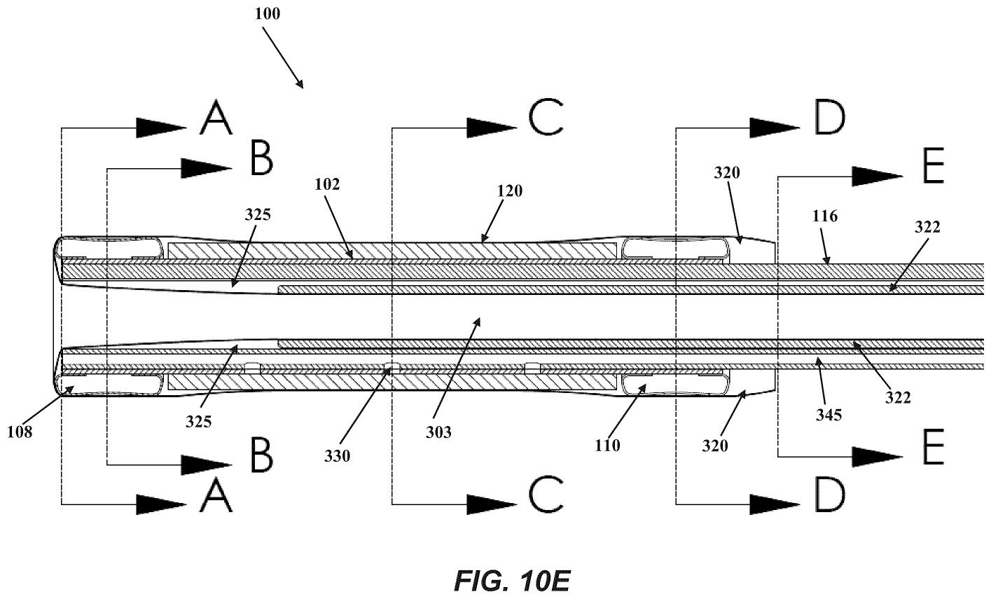

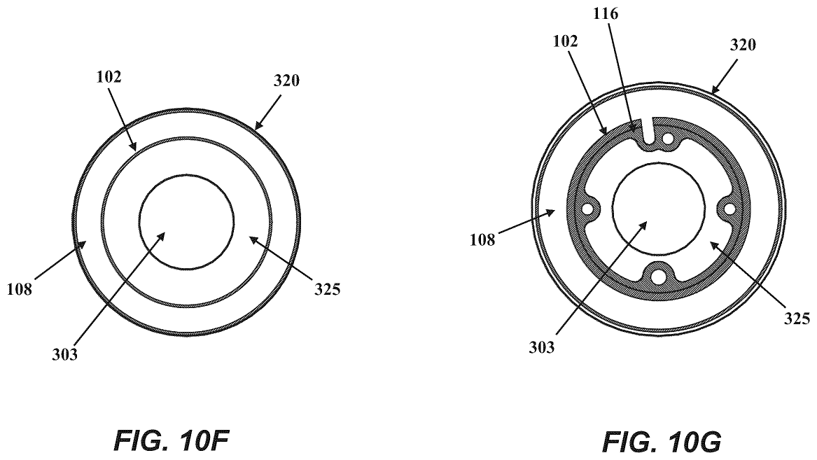

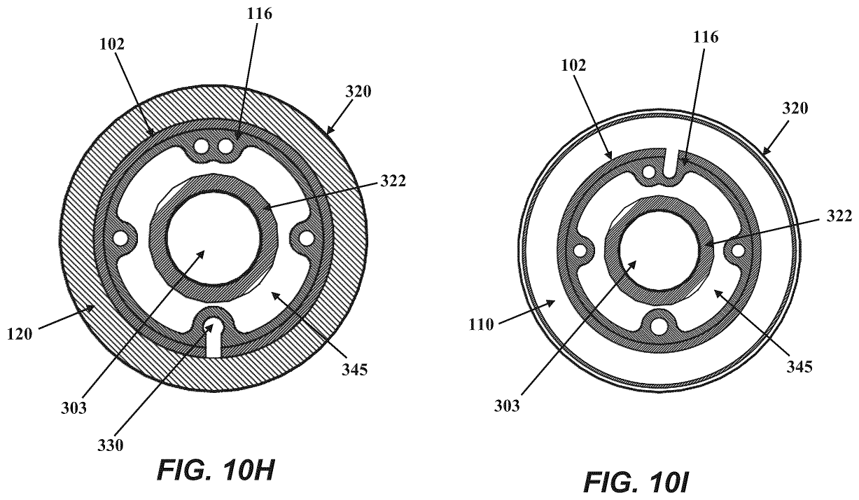

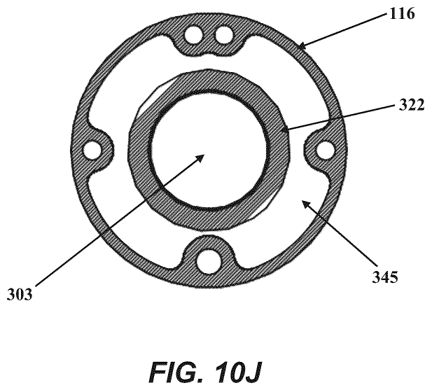

Once the device is positioned at the desired location above the area requiring isolation from fecal flow by the sleeve 220, it is detached from the delivery system, and the delivery system components including the semi-rigid tube pusher 203 and flexible membrane 204 are removed from the patient. In this regard, according to an embodiment, the flexible membrane 204 is disposed through a central lumen of the device 206 and emerges from a distal end or tip of the device 206 in order to cover and protect the expandable sealing mechanisms 212, 214 and the foam dispersed around the sleeve body. An opposite end of the flexible membrane 204 is attached to a pull handle (see, e.g., FIGS. 10A to 10D), which allows for the removal of the flexible membrane 204, by pulling the flexible membrane 204 through the central lumen, to thereby uncover and expose the expandable sealing mechanisms 212, 214 and the foam dispersed around the sleeve body. (See also, e.g., description below relating to FIGS. 10A to 10J.)

FIG. 6B illustrates the device 206 with flexible membrane or protective sheath 204 removed, and the expandable sealing mechanisms 212, 214 being inflated by filling with a fluid or inflation media (e.g., saline, mineral oil, and/or a dye-based (e.g., iodine-based) contrast solution). In some embodiments, the flexible membrane 204 is removed through the center of the semi-rigid tube pusher 203. FIG. 6C illustrates the device 208 once negative pressure is applied through the pressure lumen 210, and the expandable sealing mechanisms 212, 214 are filled with a fluid or inflation media (e.g., saline, mineral oil, and/or a dye-based (e.g., iodine-based) contrast solution). As air is removed from the space between the expandable sealing mechanisms 212, 214 on the outer surface of the sleeve body, the inner walls of the tissue cavity are drawn toward the sleeve body. As shown in FIG. 6B, the device 206 can have some or all of its components' external diameter smaller than the inner diameter of the closed off tissue cavity inner wall 215, 217 in which it is to be anchored. Once inflated, the expandable sealing mechanisms 212, 214 further create a seal with the wall of the tissue cavity at either end of the sleeve body. As shown in FIGS. 6B and 6C, once expanded, the expandable sealing mechanisms 212, 214 comprise expanded sealing elements 222, 224 that are structured to conform to the inner walls 216, 218 of the tissue cavity as negative pressure is applied, thereby creating a liquid-tight and air-tight seal. The flexibility of the expanded sealing elements 222, 224 and the angle at which they protrude allows for them to conform when negative pressure is applied to avoid creating pressure ischemia of the bowel wall. This allows the expanded sealing elements 222, 224 to lie flat against the tissue surface, creating a seal with reduced pressure on the tissue at the interface between the expandable sealing mechanisms 212, 214 (or expanded sealing elements 222, 224) and the tissue cavity wall 216, 218. The expansion of the expandable sealing mechanisms 212, 214 further provides the ability to accommodate irregularities in the contour of the inner tissue cavity wall 216, 218. In addition, the expandable sealing mechanisms 212, 214 have an external diameter that is greater than the external diameter of the foam. This allows for more reliable creation of a seal with the bowel when the bowel wall is sucked down during negative pressure activation.

The seals at both ends of the sleeve and/or the sleeve body prevent air from entering the space between the sleeve and/or the sleeve body and the cavity wall. The expandable sealing mechanism 212 at the proximal end of the sleeve body (or the sleeve) also diverts fluid and other GI content traveling through the tissue cavity into the central lumen of the sleeve in cases where the tissue cavity is the bowel. The GI content passes through the central lumen and into the sleeve 220. The GI content is thus isolated from the anastomosis more distal in the GI tract. This prevents anastomotic contamination with fecal flow. The expandable sealing mechanisms 212, 214 in combination with negative pressure create an air and fluid tight bypass of GI contents that is superior to other methods that have been used in attempts to create an effective seal at the proximal end of an intraluminal bypass sleeve.

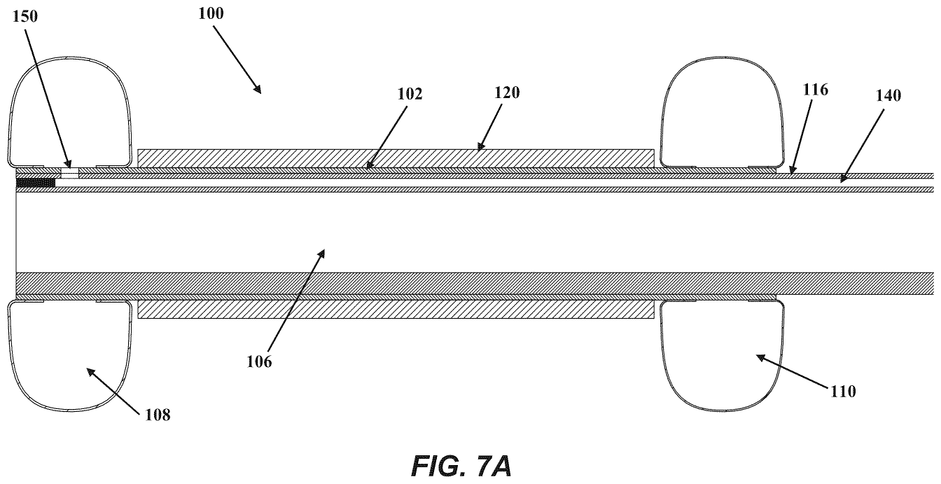

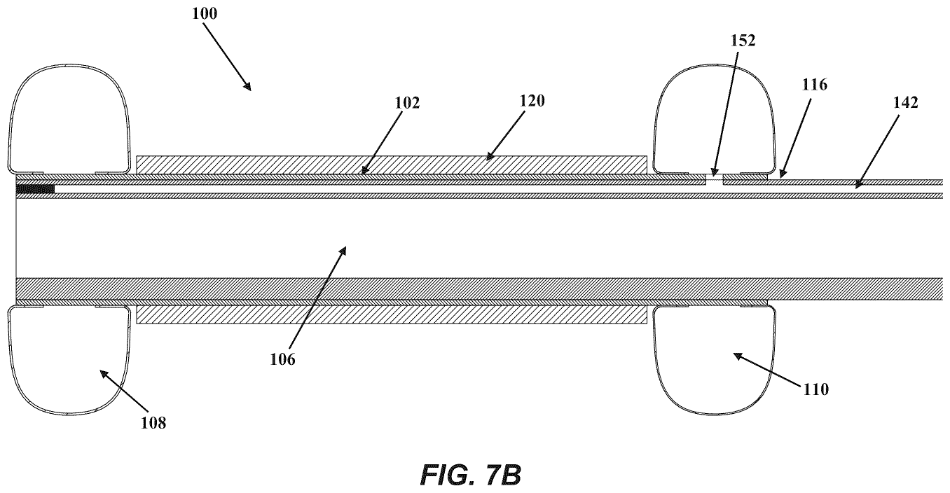

FIG. 7A shows an illustration of the anchoring system 100 of FIGS. 1A and 4A in which the expandable sealing mechanisms are in an expanded position, according to an embodiment of the invention. As shown in FIG. 7A, the anchoring system 100 includes the sleeve body 102. The first expandable sealing mechanism 108, which is in an expanded position, is disposed at a proximal end of the sleeve body 102, and the second expandable sealing mechanism 110, which is also in an expanded position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with the lumen 106. The open-cell foam 120 is disposed on the outer surface of the sleeve body 102. In the embodiment of FIG. 7A, the first expandable sealing mechanism 108 is expanded (or inflated) by filling the sealing mechanism with a fluid or inflation media, such as, e.g., saline, mineral oil, and/or a dye-based (e.g., iodine-based) contrast solution, which is delivered to the first expandable sealing mechanism 108 via the proximal fluid lumen 140 through a proximal inlet 150 in fluid communication with the first expandable sealing mechanism 108.

FIG. 7B shows an illustration of the anchoring system 100 of FIGS. 1A, 4A and 7A in which the expandable sealing mechanisms are in an expanded position, according to an embodiment of the invention. As shown in FIG. 7B, the anchoring system 100 includes the sleeve body 102. The first expandable sealing mechanism 108, which is in an expanded position, is disposed at a proximal end of the sleeve body 102, and the second expandable sealing mechanism 110, which is also in an expanded position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with the lumen 106. The open-cell foam 120 is disposed on the outer surface of the sleeve body 102. In the embodiment of FIG. 7B, the second expandable sealing mechanism 110 is expanded (or inflated) by filling the sealing mechanism with a fluid, such as, e.g., saline, mineral oil, and/or a dye-based (e.g., iodine-based) contrast solution, which is delivered to the second expandable sealing mechanism 110 via the distal fluid lumen 142 through a distal inlet 152 in fluid communication with the second expandable sealing mechanism 110.

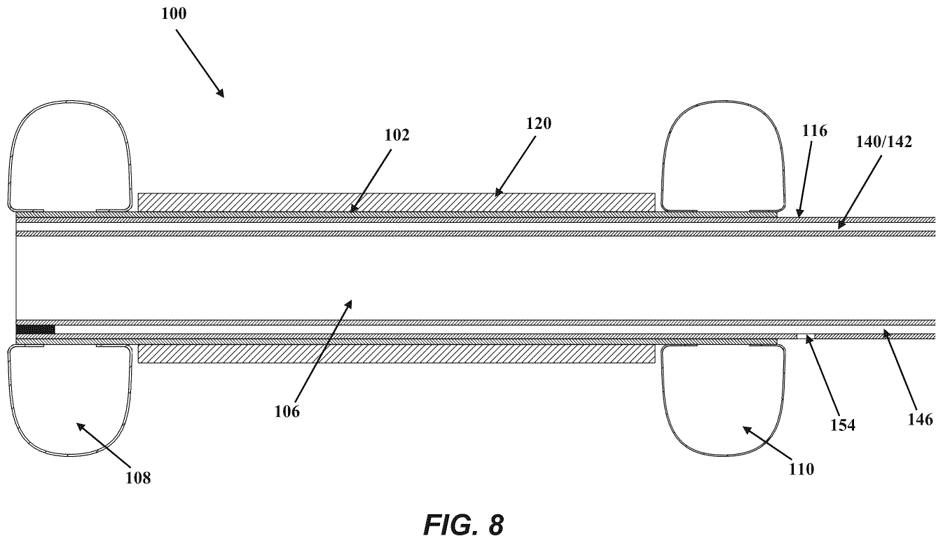

FIG. 8 shows an illustration of the anchoring system 100 of FIGS. 1A and 4A in which the expandable sealing mechanisms are in an expanded position and a contrast lumen is provided, according to an embodiment of the invention. As shown in FIG. 8, the anchoring system 100 includes the sleeve body 102. The first expandable sealing mechanism 108, which is in an expanded position, is disposed at a proximal end of the sleeve body 102, and the second expandable sealing mechanism 110, which is also in an expanded position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with the lumen 106. The open-cell foam 120 is disposed on the outer surface of the sleeve body 102. In the embodiment of FIG. 8, a contrast lumen 146 is provided in order to inject contrast dye or radiopaque dye to perform a radiographic leak test. For example, according to the embodiment of FIG. 8, the contrast lumen 146 is provided to inject contrast dye or radiopaque dye distal to the anchor portion or sleeve body 102 of the device within the bowel, via an outlet port 154, to perform a radiographic leak test.

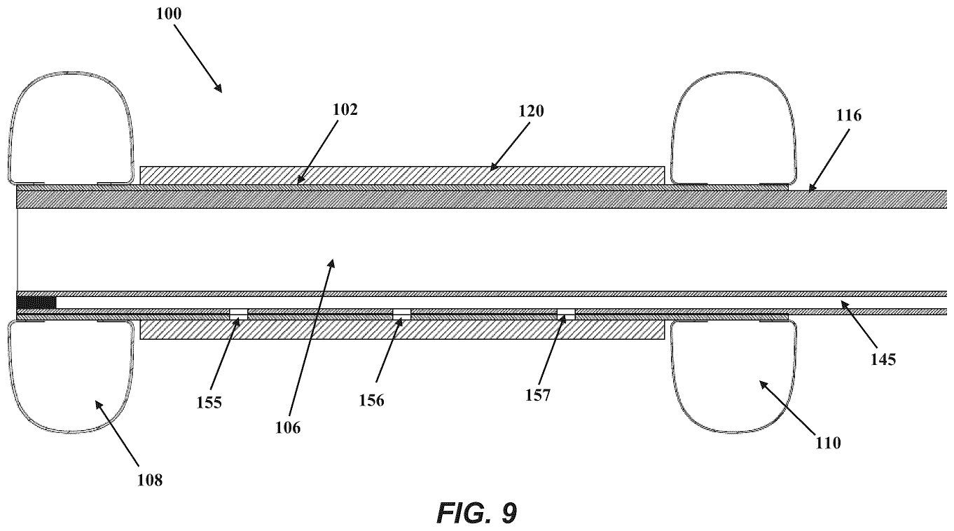

FIG. 9 shows an illustration of the anchoring system 100 of FIGS. 1A and 4A in which the expandable sealing mechanisms are in an expanded position and negative pressure is provided to the anchoring system 100, according to an embodiment of the invention. As shown in FIG. 9, the anchoring system 100 includes the sleeve body 102. The first expandable sealing mechanism 108, which is in an expanded position, is disposed at a proximal end of the sleeve body 102, and the second expandable sealing mechanism 110, which is also in an expanded position, is disposed at a distal end of the sleeve body 102. The sleeve 116 is in fluid communication with the sleeve body 102 by being connected to, coextensive with, and/or disposed within the sleeve body 102, with each of the sleeve 116 and the sleeve body 102 being in fluid connection with the lumen 106. The open-cell foam 120 is disposed on the outer surface of the sleeve body 102. In the embodiment of FIG. 9, a negative pressure lumen 145 is provided in order to apply negative pressure (e.g., vacuum) to the anchoring system 100. For example, according to the embodiment of FIG. 9, the negative pressure lumen 145 is provided to supply negative pressure to the anchor portion or sleeve body 102 of the device via outlet ports 155, 156, and 157. The application of negative pressure to the anchor portion or sleeve body 102, as well as inflation of the first and second expandable sealing mechanisms 108, 110, causes a seal to form between the first and second expandable sealing mechanisms 108, 110 and an inner surface of the tissue cavity, and creates a frictional force that resists displacement of the sleeve body 102 and/or the sleeve 116.

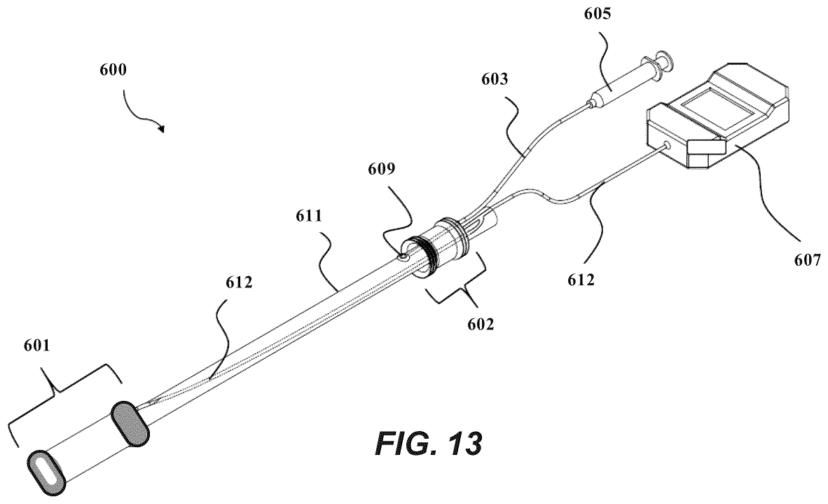

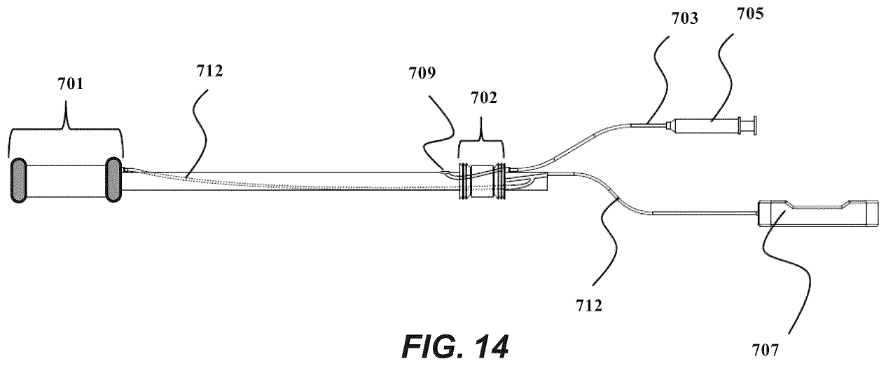

FIGS. 13 and 14 show an anchoring system for treatment of the bowel wall. In this embodiment, the anchoring system 600 has a first anchor element 601 at the proximal end of the system that is placed proximally in the bowel to an area of bowel to be treated, and a second more distal anchor element 602 that seals distally in the bowel beyond the area to be treated. There is a port 609 in fluid communication with the sealed off space between the two anchor elements and between the bowel wall and external surface of the sleeve where fluid or inflation media, such as, e.g., saline, mineral oil, and/or a dye-based (e.g., iodine-based) contrast solution, can be introduced or removed to, for example, inflate and deflate the expandable sealing mechanisms. A fluid tubing 603 in communication with the port 609 can be used to introduce fluid (e.g., saline) from a fluid infiltration source 605 such as, e.g., a syringe. This port can be accessed from outside the patient's body via the fluid tubing 603. This configuration of the device allows for delivery and removal of a fluid or inflation media (e.g., saline, mineral oil, and/or a dye-based (e.g., iodine-based) contrast solution) for inflation and/or deflation of the expandable sealing mechanisms, irrigation, drugs (such as antibiotics, anti-inflammatory drugs, or chemotherapy agents), and/or radiologic contrast between the external surface of the sleeve 611 and bowel wall between the two anchor elements 601, 602. In some embodiments, the second, more distal anchor element 602 is shorter than the first more proximal anchor element 601, since the anchoring force of the second anchor element 602 does not need to be as strong and treatment near the anal verge may be required where a longer anchor element 602 would not fit in the bowel. The system 600 further includes a pressure tube 612. The pressure tube 612 can be in fluid connection with an outer surface of the sleeve of the first anchor element 601 and the sleeve of the second anchor element 602, as shown in FIG. 13. Alternatively, the system 600 may include two pressure tubes, one for each of the first anchor element 601 and the sleeve of the second anchor element 602. The pressure tube 612 is connected to a pneumatic system 607 that is configured to apply negative pressure to the pressure tube to anchor the anchor elements 601, 602.

FIG. 14 is a side view of an embodiment of the double anchor element system, where like reference numerals as in FIG. 13 identify like features. This configuration is clinically important for a number of scenarios where treatment of an isolated segment of bowel could be beneficial. Because this configuration allows for controlled containment of a treatment agent within the bowel lumen for a discreet segment of bowel, this embodiment provides a unique ability to provide sustained and localized treatment of the bowel wall. For example, after endoscopic polypectomy, the excision site could be isolated with the disclosed embodiment and be treated with local chemotherapy. Another example might be inflammatory bowel disease, where an affected segment of bowel could have anti-inflammatory agents delivered and maintained at the site of disease. In cases of bowel wall damage or perforation, antimicrobial agents could be introduced to decrease bacterial load during healing and mitigate the risk of worsening infection. The two anchor elements can be spaced anywhere from about 1 cm to 6 feet depending on the indication and desired length of bowel to be treated. In some cases, the surgeon during an open case can advance the device manually from the outside of the bowel wall, so a very long segment of bowel could be treated and the upper limit is the length of the entire bowel. This applies to the one anchor element version of the device as well, as the device could potentially have a sleeve length that could protect the entire bowel.

There are several key distinctions of this bowel protection device from negative pressure wound therapy treatment devices that may be used within the intestine. The disclosed anchoring portion of the device is not configured to treat an area of bowel injury, wound, or anastomosis directly. It is configured for anchoring a sleeve portion of the device that protects the area of bowel injury, wound, or anastomosis. Importantly, the anchor portion of the device is designed to be positioned in healthy uninjured bowel above or proximal in the bowel from the area of bowel injury. This method dramatically increases the potential safety of this device as negative pressure is not delivered to the area of the anastomosis, damage or injury; thus, the protected area of bowel is never made ischemic or exposed to significant shear or traction forces from the device.

Negative pressure when delivered through a sponge interface to tissues has been shown to reduce the blood flow to areas where it is delivered. Thus, delivering negative pressure to the damaged area of the bowel itself can further damage the bowel or prevent healing as the blood supply of the bowel is less robust than for other tissues (especially at an area of anastomosis). Furthermore, the method and device described has a flexible sleeve that covers the area of bowel anastomosis or damage; thus, the anchoring of the device is in a separate location than the area of damaged tissue. During bowel contraction at the area of damaged bowel, there are less mechanical forces exerted on the bowel when it constricts around the device because the flexible sleeve is less mechanically rigid than a negative pressure wound therapy dressing that employ wire-stent based internal structures to maintain luminal patency and facilitate anchoring. In addition, by placing the anchor far away from the area of damaged bowel, the device does not exert mechanical force on the anastomosis or damaged tissue with traction or pulling on the device from the pressure tubing or other portions of the device that are external to the patient's body. No portion of the device is anchored distally to the damaged bowel; thus traction is only exerted on the proximal healthy bowel tissues. This further diminishes the risk of pulling apart an anastomosis repair or further injuring an area of damaged bowel.

Another difference is that the anchoring device described herein must have a much higher pullout strength as it must anchor strongly enough to maintain the entire sleeve and anchor element in position in normally functioning, uninjured bowel. To accomplish this, the anchor has to be made long enough and wide enough to allow for adequate surface area of sponge contact to prevent expulsion, the anchor and sleeve must be configured to conform to resist displacement by peristalsis, and the expandable sealing mechanism must be made more robust to prevent potential air leaks.

Unlike a device that is designed to be mechanically dislodged by bowel function and peristalsis over time, the described device is designed to stay in place over an extended period of time until it is removed by the treating clinician. The higher anchoring strength of this anchoring system 100 and more solid fixation is important because it allows for placement of the device near the site of bowel being treated. In cases of bowel anastomosis in the colon, placement of a device higher into the bowel from the anus becomes more challenging due to the curvature of the bowel. So unlike devices that must be placed much higher (>40 cm above area to be treated) in the bowel due to device migration during the treatment period, the fixed anchoring provided by the disclosed anchoring system enables the anchoring element (sleeve, expandable sealing mechanisms, and foam) to be placed only a couple of centimeters above the area to be treated. However, it may be preferable to have the anchor element placed at least 10 cm above the area to be treated to avoid any potential further damage to the vulnerable segment of bowel.

This ability to deliver controlled anchoring is achieved through the described design elements elaborated on below.

The components of the anchoring system according to some embodiments of the invention are described in detail below. Reference is made to FIGS. 1A, 2A, 3, 4A and 4B unless indicated otherwise.

### Sleeve Body

According to some embodiments of the invention, the sleeve body 102 is a flexible, concentric tube. In some embodiments, the sleeve body 102 is attached and/or bonded to the outer surface of sleeve 116 and provides localized support in the anchor section while allowing more softness and flexibility along the full length of sleeve 116. In some embodiments, the sleeve body 102 provides localized added support to prevent collapse of the sleeve 116 and the lumen 106 under expansion of the expandable sealing mechanisms 108 and 110 and added support to prevent angular displacement of the expandable sealing mechanisms 108 and 110 after expansion. The sleeve body 102 can be tailored to provide higher or lower strength by varying wall thickness and material hardness as described below.

The outer diameter and profile can be configured to move within the bowel without significant resistance when negative pressure is not being applied to the outer surface of the sleeve body 102. In some embodiments, the external diameter of the sleeve body 102 is between 11 mm and 61 mm in cross-sectional external diameter. The internal diameter of the sleeve body 102 determines the diameter of the lumen 106, and in some embodiments, the sleeve body 102 has an internal lumen diameter of between 10 mm and 60 mm in cross-sectional internal diameter. For anchoring in other tissue cavities than bowel, these parameters will differ based on the hollow viscus in which anchoring is to be achieved. In some embodiments, the sleeve body 102 may have a diameter that is greater than or equal to the diameter of the tissue cavity. In some embodiments, the sleeve 116 may have a diameter that is less than the diameter of the tissue cavity. In some embodiments, the sleeve 116 may have a diameter that is less than 95% of the diameter of the tissue cavity. In some embodiments, the sleeve 116 may have a diameter that is less than 50% of the diameter of the tissue cavity. In some embodiments, the sleeve 116 may have a diameter that is less than 25% of the diameter of the tissue cavity.

In some embodiments, the sleeve body 102 is configured to be flexible enough to be easily removed by pulling on the sleeve 116 to slide the sleeve body 102 out through the bowel and anus, but rigid enough to hold a concentric shape so that it forms a lumen 106 when negative pressure is applied. This allows for easy placement and removal of the device 100. When negative pressure is applied to the outer surface of the sleeve body 102, the sleeve body 102 and foam 120 surrounding the sleeve body 102 conform to the contours of the GI tract.

The sleeve body 102 is configured to be soft and pliable, and not to cause erosion into the bowel. The sleeve body 102 has enough flexibility and compliance to allow for the proximal and distal ends of the sleeve body 102 to conform to the bowel contours so that the foam-to-bowel wall contact can be maintained during peristalsis and the expandable sealing mechanisms 108, 110 can create and maintain a seal, yet keep the concentric tubular shape of the internal lumen 106 patent so GI contents can pass through. According to some embodiments, the sleeve body 102 comprises medical grade silicone, polyurethane, thermoplastic elastomer, rubber, or other polymer exhibiting the flexibility and rigidity properties described herein. The flexibility of the sleeve body 102 allows it to safely anchor in a patient's body because the flexibility of the sleeve body reduces pressure points created from bowel contraction forces. The sleeve body 102 according to some embodiments has a Shore A hardness between about 20 A and about 70 A to allow for maximum flexibility while maintaining a concentric form and patent lumen. The sleeve body flexibility is also determined by the body wall thickness. The sleeve body 102 is thin walled, again allowing for deformational forces to act upon it from bowel peristalsis. In some embodiments, the sleeve body 102 has a main body thickness of between 0.1 mm and 8 mm. The thinness allows for more durable materials to be utilized while continuing to accommodate peristaltic motion of the bowel wall.

The flexibility of the sleeve body 102 allows the sleeve body 102 to deform with the bowel during peristaltic motion. Peristaltic motion moves contents within the bowel by sequentially compressing the proximal section of bowel. Because the device is flexible it maintains a seal between the expandable sealing mechanisms 108, 110, even with deformation by peristalsis or passage of enteric matter. It maintains surface contact between the foam 120 and the bowel wall, and the bowel is able to compress the device without the device exerting large and potentially damaging forces on the bowel wall in return. With constant negative pressure maintenance, the negative pressure between the expandable sealing mechanisms 108, 110 creates nearer to constant normal forces during peristalsis along the length of the anchor element that prevents migration by maintaining the foam-to-bowel wall relationship. Accordingly, the device partially conforms and moves in conjunction with the bowel wall because of the distribution of the adhesive forces over the entire surface of the sleeve body 102 covered by the foam interface.

The flexibility further allows for the sleeve body 102 to maintain the position of the foam 120 on the bowel wall without creating shear forces between the foam 120 and bowel wall during bowel contractions. When the bowel contracts, the flexible sleeve body 102 deforms with the forces exerted through the attached foam 120 so the foam 120 can more easily deform with the bowel wall instead of shearing off the bowel wall resulting in device migration.

Furthermore, for a more flexible anchoring element, the peristaltic wave has less ability to push against the anchoring element due to the flexibility and conformity to the contraction. In cases of a more rigid and less conforming body such as a wire-based stent, the peristaltic wave has the resistance of the less deformable body to push against, resulting in device displacement.

Moreover, the flexibility, compressibility, and compliance of the disclosed device aids in placement and removal of the device through the curvature of the bowel lumen. The flexibility allows delivery of the device higher up in the digestive tract as the bowel becomes more tortuous and curved and allows for easy removal. This flexibility also allows for a longer sleeve body 102 that has a larger foam 120 surface area and higher resultant anchoring strength to be manipulated into the bowel. This flexibility is also important for the anchoring system 100 as the foam 120 itself has a higher friction co-efficient than that of devices without foam.

In contrast to the disclosed invention, stent-like devices have a rigidity that resists compression. This rigidity increases the normal forces on the stent and the bowel as the bowel compresses, and causes the stent to slip along the surface of the bowel along with the peristaltic wave of normal bowel contraction. Some stent-based designs do have some compressibility and flexibility, but it is much lower than that of the disclosed device. Because of the low durometer construction, thinness, compressibility, and conformability of the device sleeve body 102 according to some embodiments, the anchor element is much more resistant to displacement by peristaltic activity. The flexibility of the sleeve body 102 and the expandable sealing mechanisms 108, 110 has the further advantage compared to more rigid devices of being more easily maneuvered for placement within the bowel along the normal longitudinal curvature of the bowel lumen. Moreover, when the device is placed in a region of bowel with longitudinal curvature, it is more easily able to conform to accommodate this curvature to maintain foam-to-bowel surface area contact when negative pressure is applied and prevent pressure points that can potentially damage the bowel wall. In addition, the elimination of a wire-stent based structure greatly enhances manufacturability from both an ease and expense perspective.

The sleeve body length determines the length of the anchoring element, and the length of the anchor portion of the device is also an important characteristic of the device. The anchoring strength of the anchor portion of the device is directly dependent on the length of the sleeve body 102 and associated surface area of the foam 120 in contact with the bowel wall. Just like the diameter affects the surface area of the foam contact, so too does the length of the anchor portion. Unlike a stent, negative pressure dressing, or sheath that is located distally in the colon near the anal verge over an anastomotic site or area of damaged bowel that can be supported in place by the device rigidity and does not need to conform significantly to the bends of the intestine proximally in the bowel, the anchor portion of the system according to some embodiments is constructed in a window of lengths from greater than 3 cm to less than 25 cm in length. Our testing in the porcine model indicates that if the anchor device is less than 3 cm in length with a diameter of 33 mm, it will not have the surface area to maintain a pull-out strength of greater than 5 lbs and may be susceptible to loss of seal and low force device displacement (<5 lbs force). Furthermore, if the anchor portion of the device is longer than 25 cm in length, the device cannot easily be place around the anatomic bends of the intestine and positioned in the intended area of anchoring that is above (proximal in the bowel) the level of the area of bowel to be protected. For applications in other tissue cavities that require less pull out strength, such as a duct or esophagus, for example, the device may be shorter than 3 cm in length. Further, the embodiments of the invention are not limited to a flexible sleeve, and a stent-like sleeve surrounded in foam may also be used.

### Expandable Sealing Mechanisms

The device 100 includes expandable sealing mechanisms 108, 110 disposed at each end of the sleeve body 102 and/or along a proximal end of the sleeve 116. According to an embodiment, the expandable sealing mechanisms 108, 110 comprise two soft, inflatable elastomeric balloons that provide atraumatic sealing surfaces to both facilitate negative pressure anchoring and provide a fluid/air tight seal against fecal contamination distal to the anchoring system 100. According to one embodiment, the expandable sealing mechanisms 108, 100 are expanded by injecting a fluid or inflation media (e.g., normal saline, mineral oil, and/or a dye-based (e.g., iodine-based) contrast solution) through a syringe connected to distal and/or proximal ports of the anchoring system 100. The expandable sealing mechanisms 108, 110 contact the inner surface of the tissue cavity in which the sleeve body 102 and/or the sleeve 116 is inserted. The expandable sealing mechanisms 108, 110 serve at least two functions. First, once expanded, they create seals between the proximal and distal ends of the external surface of the sleeve body 102 with the bowel wall to create the negative pressure space where the foam 120 can suck down to the bowel wall and create anchoring forces. Second, the seals create a fluid- and air-tight seal with the inner surface of the tissue cavity at either end of the sleeve body 102 when the expandable sealing mechanisms 108, 110 are expanded and negative pressure is applied to the outer surface of the sleeve body 102 that diverts GI content through the lumen 106 of the sleeve body 102 and into the same lumen 106 of the sleeve 116 attached to the sleeve body 102. The expansion of the expandable sealing mechanisms 108, 110 minimizes the risk of fecal forward or back flow from causing disruption of the seal as fecal flow is directed towards the central lumen 106 of the sleeve body 102 by the expandable sealing mechanisms 108, 110.

The exact height of each expandable sealing mechanism 108, 110 is less important than the relationship of the sealing mechanisms to the external diameter of the foam 120 covering. The expandable sealing mechanisms 108, 110 of the system in some embodiments extend beyond the height of the foam 120 at rest so that when expansion and/or negative pressure is applied and the bowel wall collapses, a seal can be formed easily between the expandable sealing mechanisms 108, 110 and bowel wall without interference by the foam 120. Thus, the annular diameter of the expandable sealing elements 108, 110 is greater than the annular diameter of the foam 120 dispersed on the sleeve body 102 at rest when no expansion and/or negative pressure is applied. In some embodiments, the expandable sealing mechanisms 108, 110 extend at least 1 mm beyond the height of the foam 120 at rest.

The expandable sealing mechanisms 108, 110 are made of a soft, inflatable, and flexible material that allows them to be expanded and to conform to the surface of the bowel. This is important because the peristaltic forces of bowel contraction can cause potentially harmful pressure points without this flexibility. For example, the expandable sealing mechanisms 108, 110 can comprise thermoplastic elastomer, silicone, polyurethane, rubber, or other rubber-like materials or polymers. The Shore A hardness of the material can range between about 20 A and about 70 A. Similar to the low durometer of the sleeve body 102, the low durometer of the expandable sealing mechanisms 108, 110 allows for compression and conformation to the bowel lumen during the sealing process when expansion and negative pressure is applied and during bowel peristalsis. The conformability, flexibility, and compressibility of the expandable sealing mechanisms 108, 110 in a similar fashion to the flexibility of the sleeve body allow decreased displacement during peristalsis and easier device placement and removal.

The expandable sealing mechanisms 108, 110 extend radially beyond the external diameter of the sleeve body 102 and/or the sleeve 116 to form seals at each end of the sleeve body 102 and/or along a proximal end of the sleeve 116. The expandable sealing elements 180, 110 extend radially toward the inner surface of the tissue cavity. In some embodiments, the expandable sealing elements 108, 110 extend beyond the foam 120 radially, before and/or after expansion, allowing for sealing to occur at the ends of the sleeve body 102 and/or the sleeve 116 without interference from the foam 120. Each expandable sealing mechanism 108, 110 can be a single sealing element or multiple sealing elements. An expandable sealing element is an expandable element that provides for both air and fluid tight sealing and is configured to conform to the GI tissue to create one or more local air and fluid tight seals.

The expandable sealing mechanisms 108, 110 may be configured to be concentrically attached around the outer surface of the sleeve body 102 and/or the sleeve 116 or may be integrated into the wall of the sleeve body 102 and/or the sleeve 116. Specifically, this relates to the manufacturing process used to create the anchor portion, as the expandable sealing elements may be made in one mold with the sleeve body 102 and/or the sleeve 116 or they may be separately molded and adhered to the sleeve body 102 and/or the sleeve 116. According to some embodiments, the expandable sealing elements 108, 110 and sleeve body 102 and/or the sleeve 116 are created as a single molded part as both elements of the device have similar material property requirements of strength, flexibility, and conformability. In some embodiments, the sleeve body 102 and/or the sleeve 116 and the expandable sealing mechanisms 108, 110 are made from a single mold using the same material.

In some embodiments, the sleeve body 102 and/or the sleeve 116 is divided into multiple anchoring portion segments having an independent negative pressure supply. An anchoring portion segment is a section along the sleeve that independently anchors the sleeve. In some embodiments, the sleeve body 102 and/or the sleeve 116 is divided into one or more additional anchoring portion elements to create two or more sealed off areas along the sleeve that independently anchor to the bowel wall. Foam is placed between each sealed off section to distribute pressure and interface with the bowel wall. Negative pressure is applied to the spaces between the seals, while each expandable sealing mechanism is expanded, to create redundant areas of anchoring along the length of the sleeve body and/or sleeve. In some configurations, negative pressure is applied to each segment from independent negative pressure sources, while each sealing mechanism is expanded from one or more expansion and/or inflation sources. In some configurations, the segments share the same negative pressure source and/or the same inflation source. This embodiment, similar to having multiple anchoring elements, provides redundancy in the anchoring system. The advantage of this design is that if the seal is broken in one segment, there are still adhesive/anchoring forces at another segment or segments.

### Sleeve, Extension Tubing, and Collection Bag

The device 100 includes a sleeve 116 that, in some embodiments, is in fluid communication with the sleeve body 102 by being connected to, coextensive with, and/or disposed within the sleeve body 102. According to some embodiments, the sleeve 116 is directly connected to the sleeve body 102. According to some embodiments, the sleeve 116 is indirectly connected to the sleeve body 102. For example, the sleeve 116 may be connected to the second expandable sealing mechanism 110 at the distal end of the sleeve body 102. In some embodiments, the sleeve and sleeve body are a single tubular structure. For example, the sleeve 116 can also be the sleeve body 102 by having the two expandable sealing elements 108, 110 and foam 120 dispersed on the proximal end of the sleeve 116 without a separate sleeve body. The sleeve 116 shares the same lumen 106 as the sleeve body 102. The expandable sealing mechanisms 108, 110 divert GI content into the sleeve body 102. When the GI content reaches the distal end of the sleeve body 102, it continues through the lumen 106 of the sleeve 116. The sleeve 116 can have a length that is sufficient to extend from the distal end of the sleeve body 102 to a patient's anal canal, and outside the patient's body. Thus, once the GI content enters the sleeve body 102, it is directed into the sleeve 116, and is completely isolated from the inner surface of the patent's bowel distal to the sleeve body 102. The sleeve 116 forms a barrier between the GI fecal flow content and the bowel wall, thereby protecting this portion of bowel. To isolate the bowel wall from fecal flow content, the sleeve 116 should be substantially fluid impermeable. Secondarily, the sleeve 116 also mechanically shields the bowel wall from mechanical expansion forces of GI flow contents.

According to some embodiments, the sleeve 116 is bonded to the sleeve body 102 or the distal (second) expandable sealing mechanism 110. The sleeve 116 can have molded fixation attachments that are configured to lock into the sleeve body 102 or the distal sealing mechanism 110. According to some embodiments, the sleeve 116 is made of non-degradable biocompatible materials. For example, the sleeve 116 can be made of silicone, polyurethane, thermoplastic elastomer, rubber, or other polymer, though the embodiments of the invention are not limited to these materials. The sleeve 116 should be substantially impermeable to fluid and bacteria.

The sleeve 116 is configured so that its diameter allows it to dwell within the GI tract without obstructing the flow of GI flow material through it. In some embodiments, the sleeve 116 has a cross-sectional diameter of between about 10 mm and about 60 mm. The sleeve is made of an appropriate material and is thin and compliant enough so that the sleeve 116 is compressible by the bowel wall and does not eliminate the effects of peristaltic motion on fecal flow. Unlike a semi-rigid drainage tube designed primarily to maintain patency and depend on gravity and gastrointestinal flow pressures for movement of GI contents down the tube, the sleeve according to some embodiments is deformable during peristalsis to allow for serial compressions to move GI contents down the sleeve. This allows for placement of the device more proximally in the bowel, as gravity and GI flow pressure is inadequate to move material through a longer length of sleeve because resistance to flow increases with sleeve length. Furthermore, this compliance and associated flexibility allows for navigation around bowel curvatures, improves patient comfort, decreases the chance of bowel wall damage/erosion, and prevents sleeve clogging. Some embodiments of the sleeve 116 have a wall thickness of between about 50 microns and 5 mm. In some embodiments, the length of the sleeve 116 is sufficient for it to extend beyond the GI tract out of the anal canal after device placement. In some embodiments, the sleeve 116 is between about 8 inches and 72 inches in length. In some embodiments, the device is configured so that traction on the sleeve 116 from outside the body can be used to remove the device from the body cavity. The sleeve 116 must be strong enough to withstand longitudinal traction force without tearing of at least 10 lbs of force so that the sleeve can be used to extract the device after treatment is completed. The sleeve 116 in some embodiments is marked with indicators along its length that show the length of sleeve 116 residing inside of the GI tract or tissue cavity after placement in bowel or other tissue cavity. A user can use the indicators to determine whether the anchor section is migrating. The sleeve 116 according to some embodiments has a fixed length. According to some embodiments, the length of the sleeve 116 can be adjusted by cutting the sleeve 116.

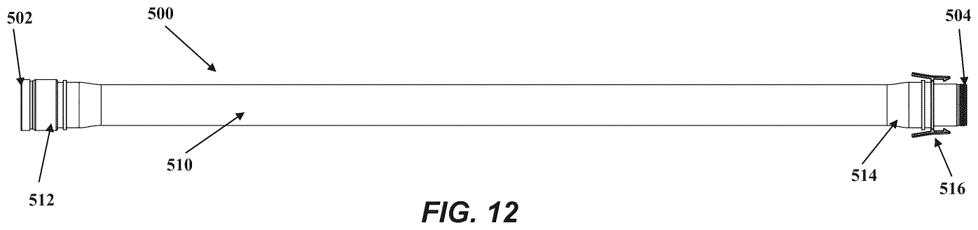

According to an embodiment, the sleeve 116 is connected or anchored to an optional extension tubing that can be attached for bedside use. FIG. 12 illustrates an extension tubing 500 according to one embodiment of the invention. As shown in FIG. 12, the extension tubing 500 includes an inlet or proximal end 502 having an inlet connection 512 that can be used to connect to the distal end of the sleeve 116. The extension tubing 500 further includes a tube 510 that extends from the inlet or proximal end 502 to an outlet or distal end 504. The outlet or distal end 504 comprises an outlet connection 514 and one or more connecting members 516 for attaching the extension tubing 500 to another member and/or a collection bag. According to one embodiment, the extension tubing 500 extends the length of the device sleeve 116 to a collection bag (as discussed below) for bedside use. According to one embodiment, the extension tubing 500 allows for the ICD device to be easily removed at bedside or in an outpatient clinic without the need for an additional invasive procedure.

According to some embodiments of the invention, a collection bag is disposed at the distal end of the sleeve 116 and/or the extension tubing 500 to manage the enteric (or GI) contents carried from the sleeve 116. The collection bag is air-tight and leakproof, and has sufficient volume to accommodate at least one day of use and a provision to empty the contents. The collection bag attaches to the sleeve 116 and/or the extension tubing (see, e.g., extension tubing 500 of FIG. 12), and is easily replaced using a quick-connect fitting(s). The collection bag is concealable, and for mobile patients, can be worn on the lower extremities of the body when used with, e.g., elastic leg straps, or can be attached to the bedside when used with infirmed patients. The collection bag is made from materials that are resistant to GI contents, provide an odor barrier, and are biocompatible for direct skin contact use. The collection bag is provided non-sterile in a single-use peel open pouch.

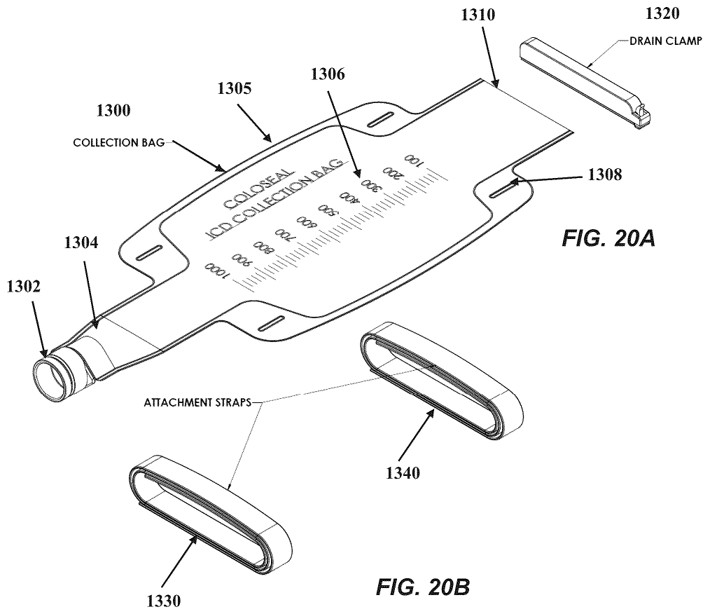

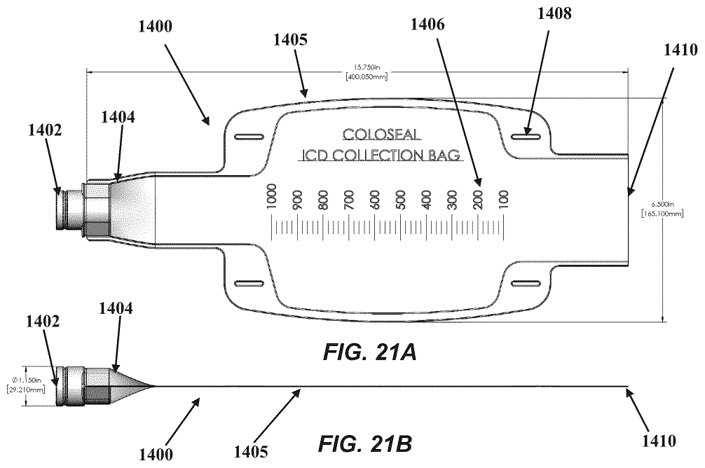

According to one embodiment, as shown in FIGS. 20A-20B and 21A-21B, the collection bag 1300, 1400, which can be attached to an end of the sleeve 116 and/or the distal end 504 of the extension tubing 500, includes an inlet end or connection 1302, 1402 that attaches to the end of the sleeve 116 and/or the distal end 504 of the extension tubing 500. The inlet end or connection 1302, 1402 of the collection bag 1300, 1400 connects to a neck portion 1304, 1404 that in turn attaches to a bag portion 1305, 1405 of the collection bag 1300, 1400 that collects GI content that flows through the sleeve body 102 and the sleeve 116. The bag portion 1305, 1405 transitions to an outlet end 1310, 1410 at which the collected GI content can be emptied from the collection bag 1300, 1400. As shown in the embodiment of FIGS. 20A-20B and 21A-21B, the bag portion 1305, 1405 tapers to the outlet end 1310, 1410. The collection bag 1300, 400 can further include a plurality of markings 1306, 1406 to measure the amount of GI content that has been collected. In addition, the collection bag 1300, 1400 can include one or more openings 1308, 1408 that allow for attaching the collection bag 1300, 1400 to the patient and/or a hospital bed.

FIG. 20A further illustrates a drain clamp 1320 that can be used to clamp or seal the outlet end 1310 of the collection bag 1300, until the collected GI content is to be emptied from the collection bag 1300 via the outlet end 1310. FIG. 20B illustrates a pair of attachment straps 1330, 1340 that can be positioned within the one or more openings 1308, 1408 of the bag portion 1305, 1405 of the collection bag 1300, 1400, in order to attach the collection bag 1300, 1400 to the patient and/or a hospital bed.

The collection bag 1300, 1400 should be substantially impermeable to air and fluid. The collection bag 1300, 1400 collects GI content that flows through the sleeve body 102 and the sleeve 116. In some configurations, the sleeve 116 ends in a port that can be kept closed for continence and opened to be emptied. In other configurations, the sleeve 116 is flexible enough to allow the anal sphincter to compress the sleeve and provide continence. In this configuration, a collection bag may not be used. According to some embodiments, the collection bag can be detached and replaced as needed. In some embodiments, the collection bag can be configured with a connection attachment that allows for cutting of the length of the sleeve and re-establishing a seal to the bag. In some embodiments, the collection bag has markings (see, e.g., markings 1306, 1406) such that the volume of effluence can be determined. In some embodiments, the collection bag has a leg strap (see, e.g., attachment straps 1330, 1340) for attaching the collection bag to the patient's body. In some embodiments, the collection bag can also contain a port to prevent any excess buildup of gasses. According to some embodiments, the external collection bag contains a one-way valve that prevents collected GI contents from flowing back into the sleeve. In some embodiments, the collection bag has elastic leg straps (see, e.g., attachment straps 1330, 1340) that fasten the collection bag to the patient's body

Foam and/or Air Conducting Rough Surface Material

The device 100 includes foam 120 that is disposed on the outer surface of the sleeve body 102. The foam 120 or foam-like material (e.g., air conducting rough surface material) serves a critical role in both increasing anchoring strength and preventing damage to the bowel. The foam 120 provides a critical friction force to hold the sleeve body 102 in place when suction is applied to the outer surface of the sleeve body 102. In addition, the foam 120 distributes negative pressure and forces to minimize pressure points that might damage the bowel.

The foam 120 dispersed on the sleeve body 102 and/or the sleeve 116 provides a high friction coefficient material with a maximum surface area where adhesion is created by the normal force created with negative pressure. Foam and/or an air conducting rough surface material is an optimal material for distributing negative pressure in this application and providing an effective coefficient of friction when negative pressure is applied. One could envision a device that uses a membrane with a series of holes placed in close proximity to form a porous membrane to distribute negative pressure. However, the normal force generated by a membrane-based device is limited by the open surface area created by the holes. In addition, the porous membrane has a much lower coefficient of friction than the rough surface of the foam and/or the air conducting rough surface material. The foam and/or the air conducting rough surface material also has a larger surface area of effective contact with the bowel due to its open cell structure and multiple pores for distributing negative pressure throughout its substance. To maintain a comparable pullout strength without foam (or an air conducting rough surface material), the magnitude of negative pressure required would have to increase and place significant point stresses on the bowel.

The foam 120 and/or the air conducting rough surface material comprises a material that is chosen to produce particular compression characteristics and coefficients of friction to prevent migration of the sleeve body 102 and/or the sleeve 116. The foam 120 and/or the air conducting rough surface material can comprise a material having a pore size that allows negative pressure to be distributed throughout the foam, while preventing ingrowth of tissue into the foam. This allows the foam 120 and/or the air conducting rough surface material to be easily dislodged from the inner surface of the tissue cavity when normal pressure is restored. In order to have the characteristics required to distribute negative pressure and create a high friction force, some embodiments of the foam 120 and/or the air conducting rough surface material have an average foam pore size between about 50 microns to about 1000 microns in diameter. The average pore size of the foam 120 and/or the air conducting rough surface material in some embodiments is between about 100 and 300 microns. The average pore size of the foam 120 and/or the air conducting rough surface material in some embodiments is between about 200 and 400 microns. The average pore size of the foam 120 and/or the air conducting rough surface material in some embodiments is between about 300 and 600 microns. Too small a pore size and the foam 120 and/or the air conducting rough surface material loses some of its friction ability and too large a pore size and the material may have tissue ingrowth and has a lower tear strength. In some embodiments, the density and material composition of the foam 120 and/or the air conducting rough surface material must allow for an overall tensile strength of the foam to be at least about 50 Kpa. This allows deforming forces and traction on the sleeve body 102 to not shear or tear the foam 120 and/or the air conducting rough surface material. Because the foam 120 and/or the air conducting rough surface material is bearing the shear force exerted on the anchoring system 100 the foam must have a high tear force that can withstand about 50 Kpa of shear force and must be fixed to the sleeve body 102 in a fashion that can withstand about 50 Kpa of distraction force without separation. The level of forces exerted on the device both from the peristaltic and expulsive forces on the sleeve body 102 and sleeve 116 are much higher than for keeping in place a piece of foam to treat a small wound area as might be done with negative pressure wound therapy.

The foam 120 and/or the air conducting rough surface material in some embodiments is comprised of a material that is hydrophilic, which can prevent the foam from drying out the surface tissue with which it comes into contact, though a hydrophobic material can also be used in some embodiments. According to some embodiments, the foam 120 and/or the air conducting rough surface material comprises polyvinyl alcohol. In some embodiments, the foam 120 and/or the air conducting rough surface material is made of polyurethane, another polymer, or organic fiber mesh. In some embodiments, the open-cell foam 120 comprises a single tubular piece of foam.

The foam 120 and/or the air conducting rough surface material covers the outer surface of the sleeve body 102 and/or the sleeve 116, and creates a friction force when negative pressure is applied to the outer surface that resists motion of the sleeve body 102 and/or the sleeve 116 with respect to the bowel. The porosity of the foam 120 and/or the air conducting rough surface material allows air to be evacuated from the region between the outer surface of the sleeve body 102 and the inner surface of the tissue without strong suction being applied to any single point. This creates a frictional force that is evenly distributed across the outer surface of the foam 120 and/or the air conducting rough surface material. The foam 120 and/or the air conducting rough surface material under negative pressure also creates a large surface area where frictional forces are created to resist dislodgement. The foam 120 and/or the air conducting rough surface material is designed to be compressible to minimize the amount of force exerted on any single point of the bowel when negative pressure is applied, and to maximize the surface area contact to the bowel wall by conforming to the shape of the bowel wall.

In some embodiments, the foam 120 and/or the air conducting rough surface material dispersed over the sleeve body 102 and/or the sleeve 116 must have a thickness or height that allows for dispersion of negative pressure around the sleeve body 102 and/or the sleeve 116 but does not extend beyond the height of the radial edge of the expandable sealing mechanisms 108, 110 at rest and/or after expansion, or results in narrowing of the sleeve lumen 106 to the point of obstructing GI content flow. If the foam 120 and/or the air conducting rough surface material is too thin, it will collapse or clog and not have enough open pores to evenly distribute negative pressure around the sleeve body 102 and/or the sleeve 116. If the foam and/or the air conducting rough surface material is too thick, it will prevent air tight seals from initiating at the expandable sealing mechanisms 108, 110 and constrict the diameter of the sleeve lumen 106. In some embodiments, the thickness of the foam 120 and/or the air conducting rough surface material disposed around the sleeve body 102 and/or the sleeve 116 is between 2 mm and 1.5 cm.

According to some embodiments, the foam 120 and/or the air conducting rough surface material can be segmented into separate subunits. In some embodiments, multiple pieces of foam are dispersed around each anchoring segment. As described above, these segments can be separated by multiple serial sealing elements. In these embodiments, negative pressure can be applied to all of the subunits in parallel or separately through independent negative pressure supplies.

Alternatives to foam may be used in some embodiments of the disclosed invention to form the interface with the bowel wall. These foam-like alternatives, which include, e.g., air conducting rough surface material, must distribute negative pressure evenly through the material, create a significant friction force to resist displacement when negative pressure is applied, have biocompatibility with the tissues of the GI tract, and compressibility and deformational properties that resist expulsion and pressure induced tissue damage. Some potential polymer-based alternatives or air conducting rough surface material are stacked mesh matrices that are wrapped around the sleeve or sleeve body, a honey-comb lattice of interconnected channels oriented in a radial fashion around the sleeve or sleeve body, or 3-D woven synthetic fabric material. Natural fiber alternatives include gauze, naturally occurring sponges, or woven fabric. However, some embodiments of this device utilize open-cell reticulated foam. According to one embodiment, the air conducting rough surface material have an average pore size of between about 200 and 400 microns. According to some embodiments, the air conducting rough surface material have a higher coefficient of friction than that of the sleeve 116 and/or the sleeve body 102.

### Pneumatic System

According to one embodiment, the device 100 includes a pressure tube (not shown) that is in fluid connection with the outer surface of the sleeve body 102 and/or the sleeve 116. The pressure tube is connected to a negative pressure source such as an air pump or vacuum that sucks air out of the tube in a controlled fashion. This pump maintains either constant or variable negative pressure at a level of pressure that allows for adequate anchoring so that the anchor portion does not become dislodged, but does not harm the bowel. The configuration of device allows for physiologically safe pressures of up to −200 mmHg, though pressures of −50 to −150 mmHg may be the preferred range of negative pressure delivery. Negative pressure is applied to the tube, after a seal is formed by expansion of the expandable sealing mechanisms 108, 110 at either end of the sleeve body 102. As the pressure tube connected to a negative pressure source continues to apply negative pressure, the inner walls of the tissue cavity are pulled toward the outer surface of the sleeve body 102 and/or the sleeve 116, bringing the tissue into contact with the foam 120. The normal force created by the negative pressure sucking down the tissue against the foam 120 creates a friction force that resists motion of the sleeve body 102. The pressure tube is configured to resist occlusion from wall collapse when negative pressure is applied. In some embodiments, there are more than one pressure tube to provide redundancy in case of kinking or clogging of any one pressure tube. In some embodiments with a plurality of pressure tubes, more flexible and compliant tubing material can be utilized due to the redundancy of negative pressure delivery. Each of these pressure tubes are individually in fluid communication with the foam 120 to allow for negative pressure delivery. In some embodiments, where there are multiple anchoring portion elements or in cases where there are multiple anchoring portion segments, there may be separate pressure tubes connected to each anchoring portion element or anchoring portion segment. The plurality of pressure tubes can be connected to a single negative pressure source such as a single pump or individually to a plurality of pressure sources such as multiple pumps.

According to an embodiment, the pressure tube (not shown) extends from the sleeve body 102 beyond the anus. The pressure tube can be disposed within the wall of the sleeve 116 or be separate. According to some embodiments, the sleeve 116 defines an additional lumen in which the pressure tube is disposed such that it is isolated from the GI content traveling through the sleeve 116. Alternatively, the pressure tube can be situated alongside the sleeve 116, either attached to the outside of the sleeve 116, inside the sleeve 116, or detached from the sleeve 116. In another embodiment, an additional lumen in the sleeve 116 is the pressure tube (see, e.g., negative pressure lumen 145 of FIG. 2C).

The proximal end of the pressure tube can be connected to the distal end of the sleeve body 102 or to the second expandable sealing mechanism 110 disposed at the distal end of the sleeve body 102.

According to some embodiments, the pressure tube is part of a pneumatic system that controls the pressure on the outer surface of the sleeve body 102 and/or the sleeve 116. The pneumatic system includes a pump that pulls air out of the pressure tube and maintains near constant and/or variable negative pressure at a set pressure level in the range of −50 mmHg to −200 mmHg. Our benchtop testing has demonstrated that −50 mmHg negative pressure to the system can produce >10 lbs longitudinal pullout force. The pneumatic pump may also in some configurations be capable of applying positive pressure, for example to assist in removal of the device from the patient's bowel. The pneumatic pump can maintain negative pressure through an electric pump mechanism or mechanical pump mechanism. The pneumatic system may include an indicator that allows the user to determine whether sufficient negative pressure has been achieved and maintained. For example, the pressure gauge can be an indicator that demonstrates that sealing is maintained as suction force is measured within the pneumatic system.

In some embodiments, the pressure tube has an adaptor that can be used to attach a syringe so that the pressure tube can be flushed and the foam 120 irrigated with fluid. This can be helpful with removal of the device from the bowel wall during the removal procedure or for flushing GI contents away from the foam interface that might clog the pneumatic system.

### Insertion and Removal

During insertion into a patient's bowel, the device 100 is introduced into the anal canal and moved past the anastomosis site, so that the second expandable sealing mechanism 110 disposed at the distal end of the sleeve body 102 is proximal to the anastomosis. The method of deployment depends on the level of the anastomosis. For low anastomosis, the device can be deployed through a capsule sheath system that is positioned manually. For higher anastomosis, an endoscope can be used to assist in the deployment. The device can be placed over the outside of an endoscope and affixed such that a user can position and deploy the device in the desired location.

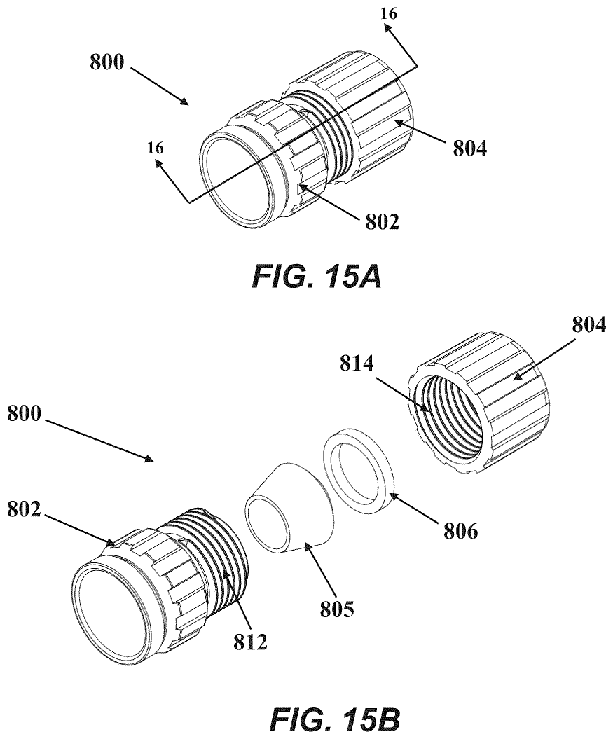

According to some embodiments, the anchoring system 100 is configured to be placed into position by an endoscope. The device 100 can have a suture or tab present that can be grasped by an endoscope grasper to pull the sleeve body 102 in place using an endoscope. In some embodiments, the device is attached to a releasable clip on the end of the endoscope that can release the device from the end of endoscope from outside the body. Alternatively, an endoscope may be used to hold a flexible member such as a wire or string attached to the anchor that is looped out of the patient's body and pulled around the fixed end of the endoscope within the bowel to pull the device into the bowel and into the desired position. As discussed in further detail below, the device 100 can be connected to an endoscope via a scope adapter (see, e.g., FIGS. 15A to 18).

In some embodiments, the sleeve 116 can be attached to the endoscope using a releasable mechanism from outside the body clamping mechanism. In some embodiments, the sleeve 116 can be attached to semi-rigid tubing that fits over the endoscope. This tubing is configured to push the anchoring system 100 into place over the endoscope and then to release from the anchoring system 100. In other embodiments, the introducing member is a first semi-rigid tube that contains the proximal portion of device. This first semi-rigid tube is advanced into the bowel through the anus, and after reaching the desired position, a second semi-rigid pushing tube that encircles the sleeve and is smaller in diameter than the first semi-rigid tube is used to hold the device in place while the first semi-rigid tube is removed. The second semi-rigid pushing tube is then removed after negative pressure anchoring of the anchoring system 100 is initiated.

FIGS. 10A-10E illustrates an ICD delivery system 300 according to one embodiment of the invention. As shown in FIG. 10A, the ICD delivery system 300 includes a handle 306, a guide shaft 303, an outer protective sheath (see, e.g., sheath 320 in FIG. 10B), and a sheath pull handle 307, which allows a physician or surgeon to place the ICD device at the time of, e.g., colon resection surgery. The ICD delivery system 300 facilitates placement of the ICD device by maintaining a small anchor profile during insertion and protecting the expandable sealing mechanisms (i.e., the expandable elastomeric balloons) from potential damage when inserting the device past a stapled anastomosis. To facilitate placement past the pelvic flexure, the guide shaft 303 may in some embodiments have a slight curve at the leading insertional end. The ICD device is inserted, advanced, and positioned with the ICD delivery system 300 transanally. An aqueous based lubricant such as, e.g., KY-Jelly is used to facilitate placement. The anchor portion is positioned 5 cm to 10 cm above (more proximal in the GI tract) the area requiring protection from fecal flow (such as the anastamosis after rectal cancer surgery). Importantly, the anchor portion of the device is designed to be placed in healthy bowel and not at the region of damaged bowel where protection is required. After positioning of the anchor, the outer sheath is removed by withdrawing it using the sheath pull handle 307. The device is then ready for deployment.

FIGS. 10B and 10C illustrate the ICD delivery system 300 of FIG. 10A in combination with the anchoring system 100 as previously described. As shown in FIGS. 10B and 10C, the anchoring system 100 includes the sleeve body 102, the first and second expandable sealing mechanisms 108, 110 disposed on the sleeve body 102, the foam 120 dispersed on the sleeve body 102, and the sleeve 116 connected to the sleeve body 102, with the sheath 320 of the ICD delivery system 300 being disposed over the proximal end of the anchoring system 100 or the sleeve body 102 in order to protect the sleeve body 102 during insertion of the anchoring system 100 into the patient. The guide shaft 303, which is attached to the handle 306, is positioned inside of the sleeve 116 and the sleeve body 102, in order to push the anchoring system 100 into position within the anal canal.

FIG. 10D illustrates a partial, expanded, cross-sectional view of the distal end or handle portion of the ICD delivery system 300 of FIGS. 10A-10C. As shown in FIG. 10D, the guide shaft 303 is attached to the handle 306, with the handle 306 having a plurality of ridges 316 to aid in the gripping of the handle 306 by a user or physician. The sheath pull handle 307 interacts with the handle 306 and is further attached to an inner portion 322 of the sheath 320 (see, e.g., FIGS. 10B and 10C) in order to remove the sheath 320 from the proximal end of the anchoring system 100 or the sleeve body 102 by pulling the sheath pull handle 307 in a distal direction.

FIG. 10E illustrates a partial, expanded, cross-sectional view of the proximal end of the ICD delivery system 300 of FIGS. 10A-10C. As shown in FIG. 10E, the sheath 320 is disposed over the proximal end of the anchoring system 100 or the sleeve body 102, which includes the first and second expandable sealing mechanisms 108, 110 and the foam 120. The guide shaft 303, which is attached to the handle 306, is positioned inside of the sleeve 116 and the sleeve body 102, in order to push the anchoring system 100 into position within the anal canal. As further shown in FIG. 10E, the sheath 320 extends to an attachment portion 325 and an inner portion 322, which are attached to the sheath pull handle 307 in order to remove the sheath 320 from the proximal end of the anchoring system 100 or the sleeve body 102 by pulling the sheath pull handle 307 in a distal direction, as discussed above. As the sheath pull handle 307 is pulled in a distal direction, the sheath 320 is removed from the outer surface of the sleeve body 102, including the first and second expandable sealing mechanisms 108, 110 and the foam 120, and pulled through the lumen 106 of the sleeve body 102 and the sleeve 116, and thereafter away from the device. This removal of the sheath 320 generally occurs once the anchoring system 100 is placed into its location, via the guide shaft 303, within the anal canal.

FIGS. 10F-10J illustrate cross-sectional views of the anchoring system with the delivery system as shown in FIG. 10E taken along various lines of FIG. 10E according to an embodiment of the invention. For example, FIG. 10F is a cross-sectional view of the anchoring system with the delivery system as shown in FIG. 10E taken along line A-A of FIG. 10E. As shown in the embodiment of FIG. 10F, this end of the anchoring system 100 with the delivery system 300 includes the sheath 320 positioned around the first expandable sealing mechanism 108, the sleeve body 102, the attachment portion 325 of the sheath 320, and the guide shaft 303.

FIG. 10G is a cross-sectional view of the anchoring system with the delivery system as shown in FIG. 10E taken along line B-B of FIG. 10E. In the embodiment of FIG. 10G, a portion of the anchoring system 100 with the delivery system 300 is illustrated that includes the sheath 320 positioned around the first expandable sealing mechanism 108, the sleeve body 102, the sleeve 116, the attachment portion 325 of the sheath 320, and the guide shaft 303.

FIG. 10H is a cross-sectional view of the anchoring system with the delivery system as shown in FIG. 10E taken along line C-C of FIG. 10E. In the embodiment of FIG. 10H, a portion of the anchoring system 100 with the delivery system 300 is illustrated that includes the sheath 320 positioned around the foam 120, the sleeve body 102, the sleeve 116, which includes an outlet 330 in one of the sleeve lumens, one of the fluid lumens 345, the inner portion 322 of the sheath 320, and the guide shaft 303.

10I is a cross-sectional view of the anchoring system with the delivery system as shown in FIG. 10E taken along line D-D of FIG. 10E. In the embodiment of FIG. 10I, a portion of the anchoring system 100 with the delivery system 300 is illustrated that includes the sheath 320 positioned around the second expandable sealing mechanism 110, the sleeve body 102, the sleeve 116, one of the fluid lumens 345, the inner portion 322 of the sheath 320, and the guide shaft 303.

10J is a cross-sectional view of the anchoring system with the delivery system as shown in FIG. 10E taken along line E-E of FIG. 10E. In the embodiment of FIG. 10J, a portion of the anchoring system 100 with the delivery system 300 is illustrated that includes the sleeve 116, one of the fluid lumens 345, the inner portion 322 of the sheath 320, and the guide shaft 303.

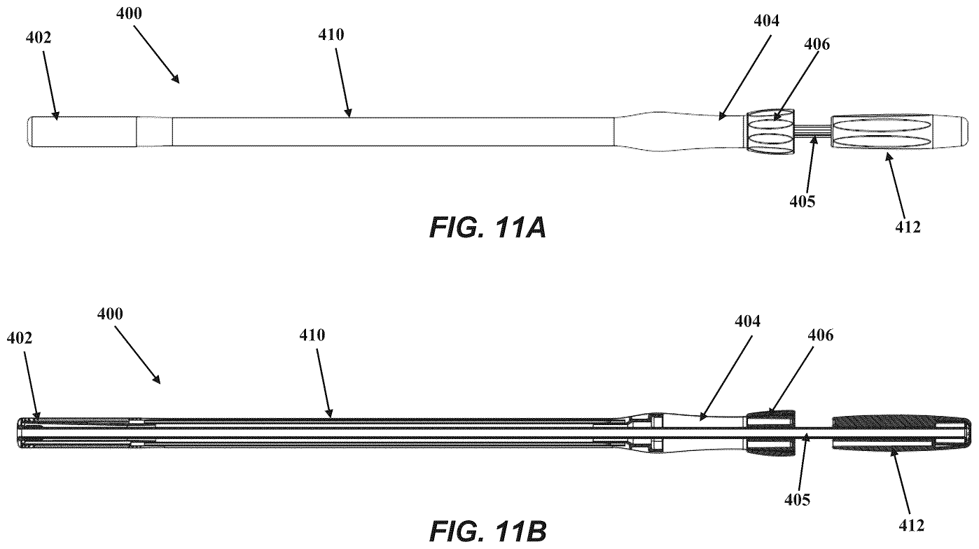

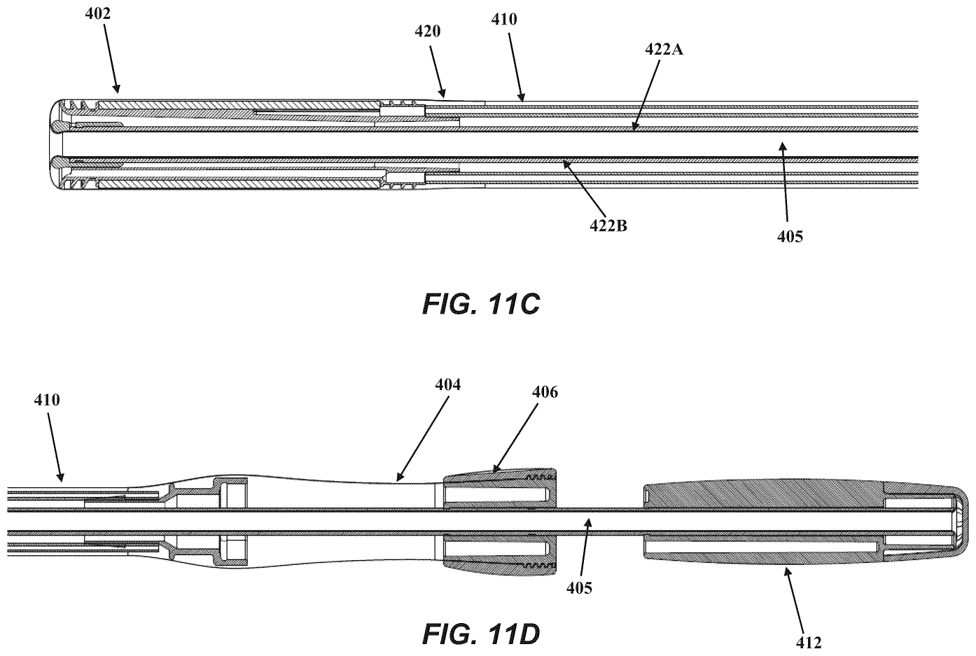

FIGS. 11A-11D illustrate an ICD delivery system 400 according to another embodiment of the invention. As shown in FIGS. 11A and 11B, the ICD delivery system 400 includes a handle portion 404, a guide shaft 405, an outer protective sheath (not shown) that extends the length of the anchoring system and/or the ICD delivery system 400 (see, e.g., sheath 420 in FIG. 11C), a connector member 406 at a distal end of the ICD delivery system 400, and a sheath pull handle 412 connected to the guide shaft 405, which allows a physician or surgeon to place the ICD device at the time of, e.g., colon resection surgery. The ICD delivery system 400 facilitates placement of the ICD device by maintaining a small anchor profile during insertion and protecting the expandable sealing mechanisms (i.e., the expandable elastomeric balloons) from potential damage when inserting the device past a stapled anastomosis. This embodiment anchors the open end of the outer sheath to the delivery handle to stretch the outer sheath to compress the anchor portion of the device. Traction on the connecting member 406 stretches the attached outer sheath and compresses the anchor portion of the device. After positioning of the device, the outer sheath is released from the connecting member 406 and withdrawn through the central lumen of the guide shaft 405. The ICD device is inserted, advanced, and positioned with the ICD delivery system 400 transanally. An aqueous based lubricant such as, e.g., KY-Jelly is used to facilitate placement. The anchor portion is positioned 5 cm to 10 cm above (more proximal in the GI tract) the area requiring protection from fecal flow (such as the anastamosis after rectal cancer surgery). Importantly, the anchor portion of the device is designed to be placed in healthy bowel and not at the region of damaged bowel where protection is required. After positioning of the anchor, the outer sheath is removed by withdrawing it using the sheath pull handle 412. The device is then ready for deployment.

FIGS. 11A and 11B further illustrate portions of an anchoring system, which engages with the ICD delivery system 400. For example, as shown in FIGS. 11A and 11B, the anchoring system includes a sleeve body 402 and a sleeve 410 connected to the sleeve body 402, with the sleeve 410 being further connected to the handle portion 404 of the ICD delivery system 400. According to one embodiment, a sheath (see, e.g., sheath 420 in FIG. 11C) of the ICD delivery system 400 is disposed over the proximal end, as well as the entire length, of the anchoring system or the sleeve body 402 and the sleeve 410 in order to protect the sleeve body 402 and the sleeve 410, as well as the first and second expandable sealing mechanisms (not shown) disposed on the sleeve body 402 and the foam (not shown) dispersed on the sleeve body 402, during insertion of the anchoring system into the patient. The guide shaft 405, which is attached to the sheath pull handle 412, is positioned inside of the sleeve 410 and the sleeve body 402, in order to push the anchoring system into position within the anal canal.

FIG. 11C illustrates a partial, expanded, cross-sectional view of the proximal end of the ICD delivery system 400 of FIGS. 11A and 11B. As shown in FIG. 11C, a sheath 420 is disposed over at least the proximal end of the anchoring system or the sleeve body 402 and the sleeve 410, which includes the first and second expandable sealing mechanisms (not shown) and the foam (not shown). The guide shaft 405, which is attached to the sheath pull handle 412, is positioned inside of the sleeve 410 and the sleeve body 402, in order to push the anchoring system into position within the anal canal. As further shown in FIG. 11C, the sheath 420 extends to an upper portion 422A and a lower portion 422B, which are attached to the sheath pull handle 412 in order to remove the sheath 420 from the anchoring system or the sleeve body 402 and the sleeve 410 by pulling the sheath pull handle 412 in a distal direction. As the sheath pull handle 412 is pulled in a distal direction, the sheath 420 is removed from the outer surface of the sleeve body 402 and the sleeve 410, including the first and second expandable sealing mechanisms (not shown) and the foam (not shown), and pulled through the lumen of the sleeve body 402 and the sleeve 410, and thereafter away from the device. This removal of the sheath 420 generally occurs once the anchoring system is placed into its location, via the guide shaft 405, within the anal canal.

FIG. 11D illustrates a partial, expanded, cross-sectional view of the distal end or handle portion of the ICD delivery system 400 of FIGS. 11A and 11B. As shown in FIG. 11D, the guide shaft 405 is attached to the sheath pull handle 412, with the guide shaft 405 being configured to slide into and out of the sleeve 410 of the anchoring system, as well as the handle portion 404 and the connector member 406 at the distal end of the ICD delivery system 400. As further shown in FIG. 11D, the handle portion 404 is configured to attach to (i) the sleeve 410 of the anchoring system at a proximal end of the handle portion 404, and (ii) the connector member 406 at a distal end of the handle portion 404.

According to some embodiments, the ICD device is deployed by (i) expanding the expandable sealing mechanisms 108, 110 (e.g., expandable balloons) by injecting a fluid or inflation media (e.g., normal saline, mineral oil, and/or a dye-based (e.g., iodine-based) contrast solution) through a syringe connected to distal and proximal ports of the device, respectively, (ii) connecting the negative pressure source (e.g., vacuum source) to a port (e.g., vacuum port) to anchor the device and/or activate negative pressure, and (iii) withdrawing the handle 306, 412 and the guide shaft 303, 405 of the ICD delivery system 300, 400 from the ICD device. According to one embodiment, a dye-based (e.g., iodine-based) contrast solution is used to fill the expandable sealing mechanisms 108, 110 (e.g., expandable balloons) as the osmotic gradient helps to maintain the expandable sealing mechanisms 108, 110 (e.g., expandable balloons) in an expanded form better and also allows for radiographic analysis of the anchor location within the body. According to an embodiment, removal of the ICD device is accomplished by (i) disconnecting the negative pressure source (e.g., vacuum source) and releasing the negative pressure or vacuum, (ii) collapsing the expandable sealing mechanisms 108, 110 (e.g., expandable balloons) by completely removing fluid through a syringe connected to distal and proximal ports of the device, respectively, (iii) injecting a small amount of normal saline through a port (e.g., vacuum port) to break the anchoring seal, and (iv) removing an external dressing (discussed in further detail below) and gently pulling the ICD device sleeve until the ICD device is fully removed from the anal verge.

In some embodiments, there is a releasable, fluid-tight, and detachable connector that allows for removal of a length of the sleeve outside of the body to allow for more easy delivery of the device. In some embodiments, the connector is located at 8 inches to 36 inches from the closest sealing mechanism. In other embodiments, the sleeve is directly connected to the effluence bag or left open at 8 inches to 36 inches from the closest sealing mechanism 110.

According to some embodiments, the device 100 has a removal system that allows it to be removed as needed. Fluid (e.g., saline) or positive pressure can be delivered down the pressure tube (not shown) and/or the negative pressure lumen 145 to reduce the adhesive force created to anchor the device 100. The device 100 can then be safely removed from the patient. In some embodiments, the device 100 is configured with a port so that fluid (e.g., a saline solution) can be used to infiltrate tubing in communication with the foam and detach the sleeve body 102 from the bowel wall. The fluid can be introduced into the pressure tube (not shown) or the device 100 can have a separate tube that extends outside the patient's body to provide irrigation (see also, e.g., flushing lumen 144 of FIG. 2C). It may be preferable to use the pressure tubing for both negative pressure delivery and irrigation. In some embodiments, the irrigation system is in fluid connection with the pressure tube, wherein the irrigation system introduces a fluid into the pressure tube for irrigation. The irrigation through the tube can be used to wash out abdominal contents that may have leaked around the proximal, expandable sealing mechanism 108 and to detach the device 100 from the patient's bowel wall. By use of one or more of these removal methods, the pullout force becomes negligible and the device 100 can be removed without damaging the surrounding tissue.

### Scope Adapter

According to some embodiments, the system includes an ICD scope adapter that can be used in conjunction with the ICD device. The scope adapter allows for insertion and deployment of the ICD device with a sigmoidoscope, endoscope, or colonoscopy scope.

FIGS. 15A and 15B illustrate an ICD scope adapter according to one embodiment of the invention. As shown in FIGS. 15A and 15B, the scope adapter 800 includes a main body 802, a compression nut 804, a compression sleeve 805, and a compression washer 806. As further shown in FIG. 15B, the main body 802 includes a plurality of threads 812 configured to engage with a plurality of grooves 814 provided along an interior surface of the compression nut 804.

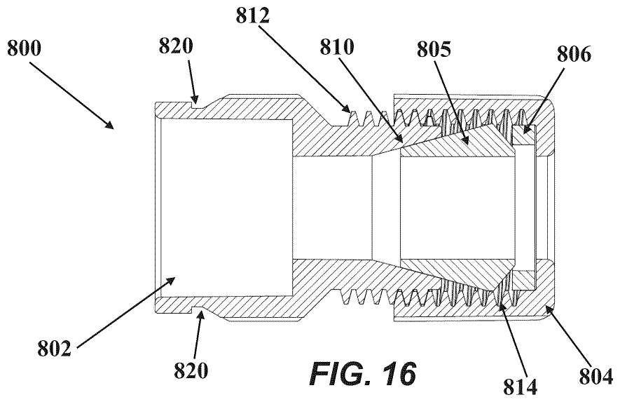

FIG. 16 illustrates a cross-sectional view of the scope adapter shown in FIGS. 15A and 15B taken along line 16-16 of FIG. 15A according to an embodiment of the invention. As shown in FIG. 16, the main body 802 of the scope adapter 800 engages with the compression nut 804 via the plurality of threads 812 provided on the main body 802 engaging with the plurality of grooves 814 provided on the compression nut 804. As further shown in FIG. 16, the compression sleeve 805 has a sliding taper 810 that allows for the compression sleeve 805 to easily fit inside of the main body 802. The compression washer 806 is disposed within the compression nut 804 and presses against the compression sleeve 805. In the embodiment of FIG. 16, the main body 802 further includes a recess 820 on each side of the main body 802 that allows for connection with a sigmoidoscope, endoscope, or colonoscopy scope, as discussed in more detail below.

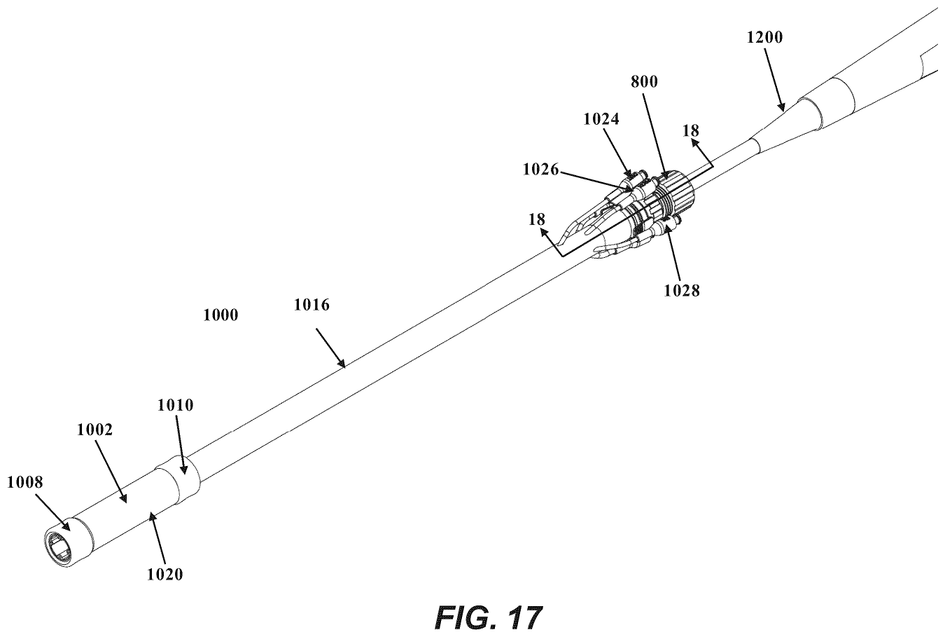

FIG. 17 is a schematic illustration of an anchoring system attached to an endoscope via a scope adapter according to some embodiments of the invention. As shown in the embodiment of FIG. 17, an anchoring system 1000 is provided that includes a first expandable sealing mechanism 1008 disposed at a proximal end of the sleeve body 1002, a second expandable sealing mechanism 1010 disposed at a distal end of the sleeve body 1002, an open cell foam 1020 disposed on the outer surface of the sleeve body 1002, and a sleeve 1016 that is in fluid communication with the distal end of the sleeve body 1002. The anchoring system 1000 further includes a plurality of connectors or ports 1024, 1026, and 1028 for providing a fluid or inflation media, e.g., saline, mineral oil, and/or a dye-based (e.g., iodine-based) contrast solution, for expansion of the first and second expandable sealing mechanisms 1008, 1010, irrigation, drugs (such as antibiotics, anti-inflammatory drugs, or chemotherapy agents), and/or radiologic contrast, as discussed above with respect to FIG. 1A. As further shown in FIG. 17, the scope adapter 800 of the embodiment of FIGS. 15A, 15B, and 16 is provided to connect an end (i.e., distal end) of the anchoring system 1000 to an endoscope 1200.

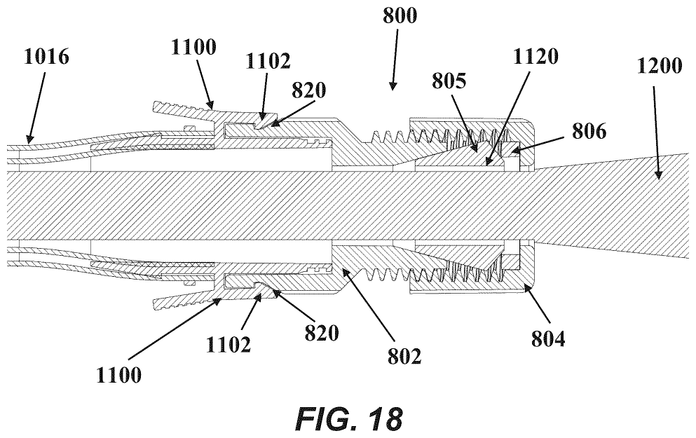

FIG. 18 is a cross-sectional view of the scope adapter shown in FIG. 17 taken along line 18-18 of FIG. 17 according to an embodiment of the invention. As shown in FIG. 18, the sleeve 1016 of the anchoring system 1000 of FIG. 17 attaches to the scope adapter 800 via a quick connector latch point or fitting, which includes an extension member 1100 having a latching member 1102 that is provided on the sleeve 1016 which latches with the recesses 820 provided on each side of the main body 802 of the scope adapter 800. According to one embodiment, the ICD scope adapter 800 attaches to the quick connector latch point or fitting of the ICD device (e.g., anchoring system 1000), such that the ICD device attaches to the ICD scope adapter 800 by sliding over the shaft of the endoscope 1200 (or sigmoidoscope or colonoscopy scope) (see, e.g., scope-shaft interface 1120 of FIG. 18) and is thereafter positioned according to physician needs or preference. When positioned at the desired location, the main body 802 of the ICD scope adapter 800 is held firmly with one hand, while the compression nut 804 is turned in a clockwise direction with the other hand. The compression nut 804 of the ICD scope adapter 800 is turned until the compression sleeve 805 grips the outside of the shaft of the endoscope 1200 (or sigmoidoscope or colonoscopy scope) to provide a non-slip attachment (see, e.g., scope-shaft interface 1120 of FIG. 18).

According to an embodiment, the ICD device (e.g., anchoring system 1000 of FIG. 17) is prepared for placement within a patient. If the provider wishes to advance the end of the endoscope 1200 more distally or retract it more proximally, the provider simply loosens the compression nut 804 of the ICD scope adapter 800 and can adjust the position of the compression sleeve 805 of the ICD scope adapter 800 along the endoscope 1200 to the desired position prior to retightening.

### ICD Dressing

According to some embodiments, the system includes an ICD retention dressing that provides additional means to secure the ICD device to the patient and minimize migration. The ICD retention dressing can be provided in an easy open cold-seal pouch.

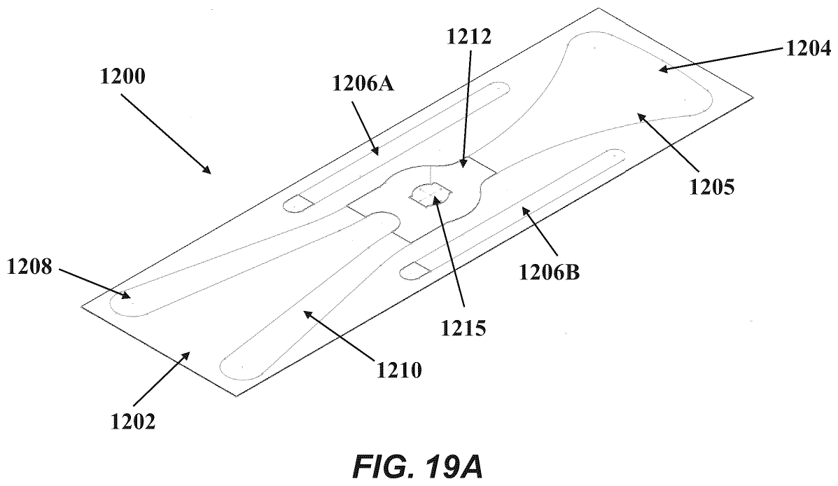

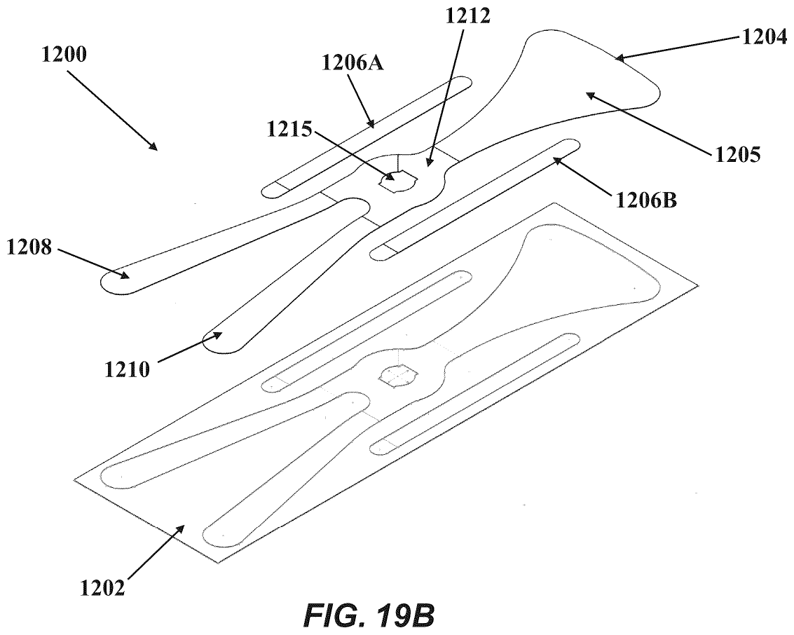

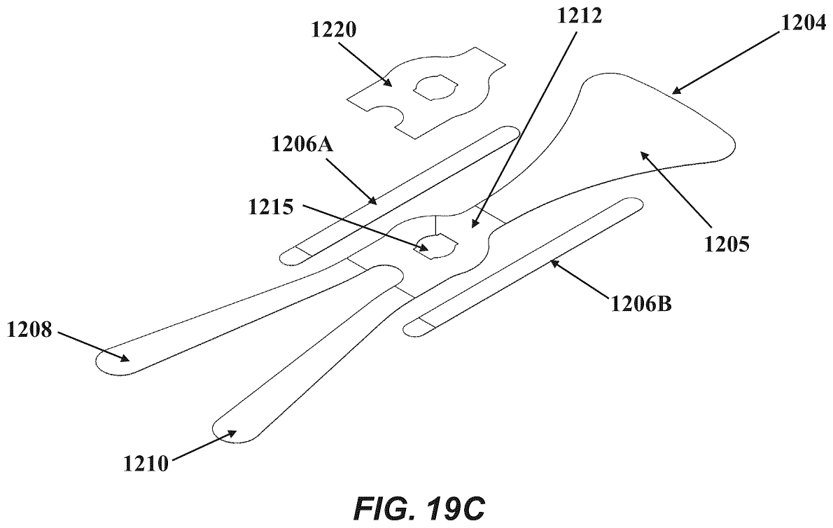

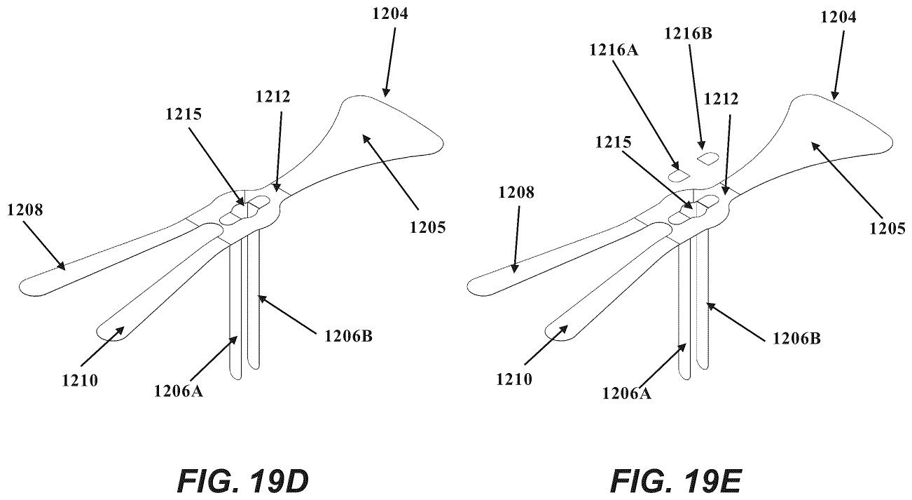

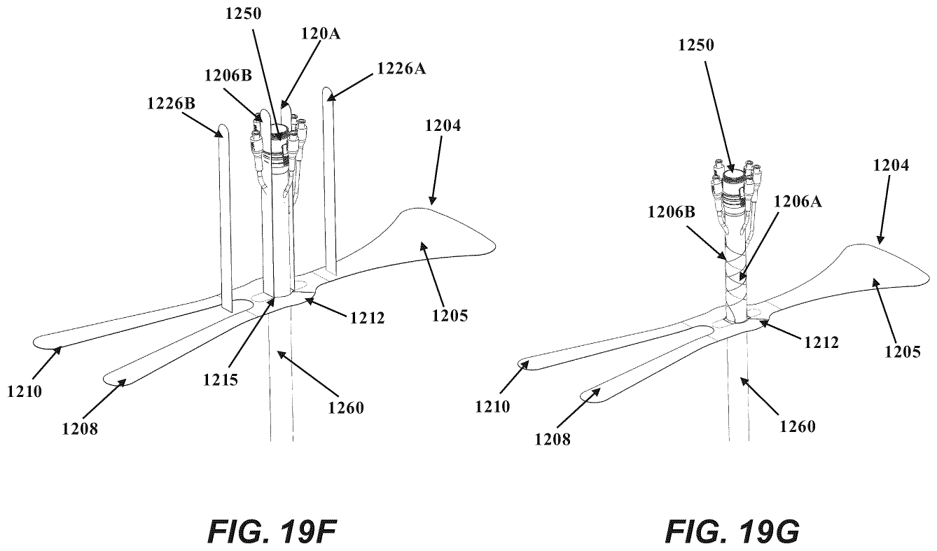

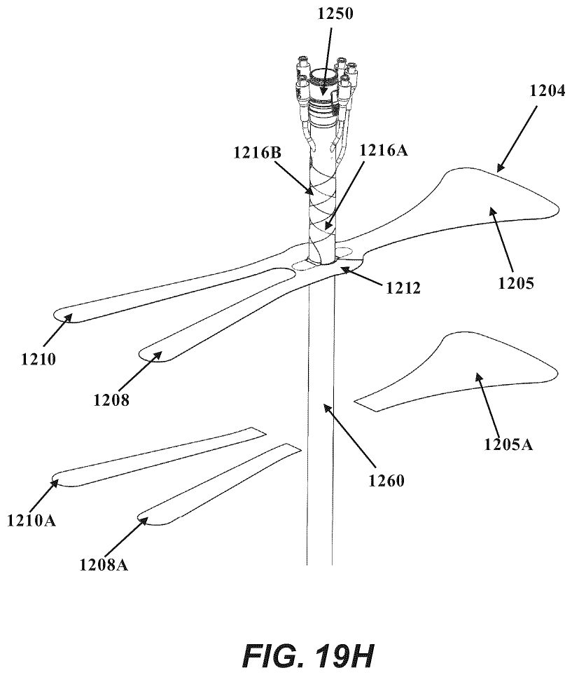

FIGS. 19A-19H illustrate an ICD retention dressing according to one embodiment of the invention. As shown in FIG. 19A, the ICD retention dressing 1200 includes a retention dressing 1204 and two retention dressing straps 1206A, 1206B. The retention dressing 1204 and retention dressing straps 1206A, 1206B are precut on a carrier sheet 1202 that can be prepared from, e.g., mylar, and further include pre-cut removable liner paper to aid in attachment (as discussed in further detail below). As further shown in FIG. 19A, the retention dressing 1204 includes a body portion 1205, a middle section 1212 having a circular opening 1215, a first leg member 1208, and a second leg member 1210.

FIGS. 19B-19H illustrate a method to attach the ICD retention dressing shown in FIG. 19A to an anchoring system according to an embodiment of the invention. In a first step, as shown in FIG. 19B, the ICD retention dressing 1200, which includes the retention dressing 1204 and the two retention dressing straps 1206A, 1206B, is removed from the carrier sheet 1202. In a second step, as shown in FIG. 19C, a first pre-cut removable liner 1220 is removed from the middle section 1212 of the retention dressing 1204, which includes the circular opening 1215. Thereafter, in a third step, as shown in FIG. 19D, the two retention dressing straps 1206A, 1206B are positioned within the circular opening 1215 of the middle section 1212 of the retention dressing 1204. In a fourth step, as shown in FIG. 19E, second and third pre-cut removable liners 1216A, 1216B are removed from ends of the two retention dressing straps 1206A, 1206B, respectively, in order to attach the two retention dressing straps 1206A, 1206B to the circular opening 1215 of the middle section 1212. In a fifth step, as shown in FIG. 19F, fourth and fifth pre-cut removable liners 1226A, 1226B are removed from sides of the two retention dressing straps 1206A, 1206B, respectively. In addition, in the fifth step, as shown in FIG. 19F, an anchoring device or ICD device 1260 is inserted into the circular opening 1215 of the middle section 1212 of the retention dressing 1204, such that an end 1250 of the anchoring device or ICD device 1260 is positioned between the two retention dressing straps 1206A, 1206B. In a sixth step, as shown in FIG. 19G, the two retention dressing straps 1206A, 1206B are wrapped (e.g., spiral wrapped) around the anchoring device or ICD device 1260. The two opposed retention dressing straps 1206A, 1206B spiral wrap and adhere to the anchoring device or ICD device 1260, in particular the sleeve body of the anchoring device or ICD device 1260, in order to secure the anchoring device or ICD device 1260 to the ICD retention dressing 1200. Finally, in a last step, as shown in FIG. 19H sixth and seventh pre-cut removable liners 1208A, 1210A are removed from the first leg member 1208 and the second leg member 1210 of the retention dressing 1204, respectively, and an eighth pre-cut removable liner 1205A is removed from the body portion 1205 of the retention dressing 1204. At this point, the ICD retention dressing 1200 is applied to a patient's inner buttocks via the body portion 1205 of the retention dressing 1204, and wraps around the patient to the lower back and to the front groin area. According to one embodiment, the first leg member 1208 and the second leg member 1210 of the retention dressing 1204 attach to a front side of the patient and are disposed on either side of their front groin area, while the middle section 1212 of the retention dressing 1204 having the circular opening 1215 is positioned around the anus of the patient.

According to an embodiment, the material of the ICD retention dressing is a biocompatible, clear polyurethane film laminated to a spunlaced polyester non-woven fabric, such that the inner surface is coated with a medical grade pressure sensitive acrylic adhesive and supplied on a removable paper liner. The medical grade adhesive can be designed for extended-wear skin contact use. In addition, the outer spunlaced polyester surface can have a thin non-absorbent coating.

According to another embodiment, the ICD retention dressing is designed to be attached to the skin and worn by the patient for up to, at least, 14 days.

### ICD Device Column Strength

According to some embodiments of the invention, the ability of the anchoring section of the ICD device to move forward against an external resistance is due to the combined effect of (i) the column strength for the diameter, wall thickness, shape, and material type of the outer sleeve, and (ii) the internal support provided by the diameter, wall thickness, shape, and material type for the internal supporting member (i.e., guide shaft). In this case, the relevant yield strength for column buckling is the compressive yield strength of the chosen material. The column strength of the outer sleeve can be calculated and tailored to the application by proper selection of the material type and material properties. In addition to column strength of the outer sleeve, the internal support (guide shaft) prevents collapse and buckling both axially and laterally. The added support allows use of a softer and/or thinner wall material, while providing the strength needed to overcome the forward resistance needed to insert the device.

According to some embodiments, the outer sleeve (e.g., sleeve 116) has a column strength of 4.0 lbs., when supported by an internal support member. According to another embodiment, the column strength may range from 3.0 lbs. to 6.0 lbs.

According to some embodiments, the outer sleeve (e.g., sleeve 116) has a wall thickness of 0.035 inches, although the wall thickness may range from 0.020 inches to 0.080 inches in thickness. According to one embodiment, the outer sleeve has a hardness of Shore 60 A durometer, although the hardness may range from Shore 40 A to Shore 90 A.

According to some embodiments, the internal support member (guide shaft) has a diameter of 0.500 inches, although the diameter may range from 0.375 inches to 0.625 inches. According to an embodiment, the internal support member has a wall thickness of 0.06 inches, although the wall thickness may range from 0.020 inches to 0.080 inches in thickness. According to an embodiment, the internal support member material has a stiffness of 1,600 MPa Tensile E modulus, although the stiffness may range from 220 to 2,500 MPa Tensile E modulus.

The embodiments of the invention described herein provide an anchoring device that is designed to have redundant stool bypass, even if negative pressure fails, because the expandable sealing mechanisms (e.g., balloons) still will block most fecal flow and the device will stay in place with the external fixation due to the column strength of the sleeve and/or the sleeve body. In addition, the use of the device on an endoscope requires that the sleeve and/or the sleeve body will not collapse in order for the device to be pushed into the patient's body. Thus, a certain column strength is necessary in order to ensure the delivery system using the scope adapter will work.

### Other Uses

The embodiments of the invention described herein may have uses outside of protection of damaged bowel or anastomosis protection. For example, the disclosed device and method may also be used for continence control in settings like an Intensive Care Unit. In these settings, fecal contamination of the perineum can result in significant skin irritation and breakdown. Existing continence control devices for diverting fecal flow into a collection bag often result in complications such as fecal leaks, displacement of fecal tubes, and erosion into the bowel wall. In contrast, the device and method described here can anchor a fecal collection sheath/sleeve within the rectum of a patient with an anchoring mechanism that is non-traumatic, sealed off from leakage, not easily dislodged, and easily reversible. The anchoring methods described herein may also be used to fixate other sheaths/sleeves or drug delivery devices within the bowel. For example, sheaths/sleeves for limiting absorption used for treating metabolic disorders, diabetes, or obesity may be anchored using the described technique. Specialized sheaths/sleeves designed to elute drugs may also be anchored using the described technique. For example, a sheath/sleeve attached to the anchor device described herein can contain controlled release anti-inflammatory drugs to treat inflammatory bowel disease. Moreover, as described above, a second anchor element can be placed distally to create a sealed space between the treated segment of bowel, the two anchor elements, and the sleeve. This space can be filled with therapeutic solutions such as antibiotics, anti-inflammatory drugs, or chemotherapeutic agents for cancer. This allows for controlled local delivery to a segment of bowel wall isolated between the two anchor elements. Also, as previously mentioned, sleeves may be anchored that may help with diverting flow from a damaged segment of bowel such as a perforation within the bowel, ischemic bowel, bowel contused by blunt trauma, or bowel that is inflamed or dilated such as in cases of inflammatory bowel disease.

Further aspects of the present disclosure are provided by the subject matter of the following clauses.

An anchoring system comprising a sleeve having an inner surface defining a lumen, a first expandable sealing mechanism disposed along a proximal end of the sleeve, a second expandable sealing mechanism disposed along the proximal end of the sleeve, and an open-cell foam disposed on the outer surface of the sleeve, wherein expansion of the first and second expandable sealing mechanisms and application of negative pressure to the anchoring system, causes a seal to form between the first and second expandable sealing mechanisms, the outer surface of the sleeve, and an inner surface of a tissue cavity.

The anchoring system of any preceding clause, further comprising a sleeve body, wherein (i) the first expandable sealing mechanism is disposed at a proximal end of the sleeve body and (ii) the second expandable sealing mechanism is disposed at a distal end of the sleeve body.

The anchoring system of any preceding clause, wherein the distal end of the sleeve body is connected to the proximal end of the sleeve.

The anchoring system of any preceding clause, wherein the sleeve body is coextensive with the sleeve.

The anchoring system of any preceding clause, wherein the sleeve body is disposed on the proximal end of the sleeve.

The anchoring system of any preceding clause, wherein the sleeve includes a plurality of fluid lumens.

The anchoring system of any preceding clause, wherein the plurality of fluid lumens includes one or more of (i) a distal fluid lumen to provide a fluid to the first expandable sealing mechanism, (ii) a proximal fluid lumen to provide a fluid to the second expandable sealing mechanism, (iii) a flushing lumen, (iv) a contrast dye lumen, and (v) a negative pressure lumen.

The anchoring system of any preceding clause, wherein the application of negative pressure creates a frictional force that resists displacement of the sleeve.

The anchoring system of any preceding clause, wherein the application of negative pressure brings the open-cell foam disposed on the outer surface of the sleeve into contact with the inner surface of the tissue cavity thereby creating the frictional force that resists displacement of the sleeve.

The anchoring system of any preceding clause, wherein the first and second expandable sealing mechanisms are expanded by providing a non-compressible fluid to expand the first and second expandable sealing mechanisms.

The anchoring system of any preceding clause, wherein the non-compressible fluid is at least one of saline, mineral oil, and a dye-based contrast solution.

The anchoring system of any preceding clause, wherein, once expanded, the first and second expandable sealing mechanisms form a substantially airtight and fluid-tight seal with the inner surface of the tissue cavity.

The anchoring system of any preceding clause, wherein the sleeve protects the inner surface of the tissue cavity from fecal flow.

The anchoring system of any preceding clause, wherein the lumen has a diameter between approximately 1 cm and approximately 6 cm.

The anchoring system of any preceding clause, herein the sleeve comprises a flexible material having a Shore A hardness between about 20 A and about 70 A.

The anchoring system of any preceding clause, wherein the open-cell foam comprises polyvinyl alcohol, polyurethane foam, or other synthetic polymer.

The anchoring system of any preceding clause, wherein the open-cell foam has a tensile strength of at least 50 kpa.

The anchoring system of any preceding clause, wherein the open-cell foam has a thickness of between 2 mm and 150 mm.

The anchoring system of any preceding clause, wherein the first and second expandable sealing mechanisms each comprises an expandable elastomeric balloon.

The anchoring system of any preceding clause, wherein the first and second expandable sealing mechanisms each comprises multiple expandable elastomeric balloons.

The anchoring system of any preceding clause, wherein (i) the first expandable sealing mechanism comprises a single expandable elastomeric balloon, and (ii) the second expandable sealing mechanism comprises multiple expandable elastomeric balloons.

The anchoring system of any preceding clause, wherein the first and second expandable sealing mechanisms have an annular diameter that is greater than an annular diameter of the open-cell foam dispersed around the sleeve.

The anchoring system of any preceding clause, wherein the sleeve has a column strength of about 3.0 lbs. to 6.0 lbs.

The anchoring system of any preceding clause, further comprising a collection bag that is disposed at a distal end of the sleeve and configured to manage enteric contents carried from the anchoring system.

The anchoring system of any preceding clause, further comprising a retention dressing to attach the anchoring system to skin.

The anchoring system of any preceding clause, further comprising an extension tubing connected to a distal end of the sleeve, the extension tubing configured to extend the anchoring system outside of a patient.

The anchoring system of any preceding clause, further comprising a scope adapter configured to connect the sleeve to at least one of an endoscope, a sigmoidoscope, or a colonoscope.

The anchoring system of any preceding clause, wherein the scope adapter comprises a main body, a compression nut, a compression sleeve, and a compression washer.

The anchoring system of any preceding clause, wherein the main body includes a plurality of threads configured to engage with a plurality of grooves provided along an interior surface of the compression nut.

The anchoring system of any preceding clause, further comprising a connector latch point configured to connect the sleeve to at least one of an endoscope, a sigmoidoscope, or a colonoscopy scope via the scope adapter.

The anchoring system of any preceding clause, further comprising a negative pressure source, wherein negative pressure is applied to the anchoring system by the negative pressure source to maintain one of a constant negative pressure or a variable negative pressure at a level between −50 mmHg and −200 mmHg.

The anchoring system of any preceding clause, further comprising a negative pressure lumen configured to provide negative pressure to the sleeve.

The anchoring system of any preceding clause, wherein the sleeve has a length that allows it to extend outside the tissue cavity.

The anchoring system of any preceding clause, wherein the lumen and the first and second expandable sealing mechanisms are compressible by normal peristaltic forces of a patient's bowel.

The anchoring system of any preceding clause, wherein the sleeve has a length that is between about 3 cm and about 25 cm.

The anchoring system of any preceding clause, wherein the sleeve and/or the sleeve body are comprised of one or more of silicone, polyurethane, thermoplastic elastomer, rubber, rubber-like material, or other polymer.

The anchoring system of any preceding clause, wherein the sleeve and the sleeve body are comprised of one continuous tubular extrusion.

The anchoring system of any preceding clause, wherein the proximal end of the sleeve is in sealed fluid communication with, and extending distal to, the distal end of the sleeve body and the second expandable sealing mechanism, wherein the proximal end of the sleeve, extending distal to the distal end of the sleeve body, is configured to cover and protect a damaged area of tissue of the tissue cavity from content flowing through the lumen of the sleeve and the sleeve body.

A delivery system comprising an outer protective sheath that encases the anchoring system of any preceding clause, a handle, a guide shaft, and a sheath pull handle, wherein the anchoring system is configured to be pushed into position by advancing the guide shaft into a patient's bowel.

A delivery system comprising a handle, a guide shaft connected to the handle, an outer protective sheath configured to cover and protect a proximal end of an anchoring system having at least one expandable sealing mechanism, and a sheath pull handle attached to the outer protective sheath.

The delivery system of any preceding clause, wherein the outer protective sheath is disposed within a central lumen of the guide shaft and is configured to extend from the central lumen in order to cover and protect the proximal end of the anchoring system having the at least one expandable sealing mechanism.

The delivery system of any preceding clause that is used with an anchoring system having a sleeve with a column strength that is high enough to prevent collapse of the system during insertion via the delivery system

A method for anchoring a sleeve in a tissue cavity, the sleeve having an outer surface comprising foam for contacting an inner wall of the tissue cavity, and an expandable sealing mechanism for isolating a portion of the tissue cavity adjacent to the sleeve from a remainder of the tissue cavity, the method comprising inserting the sleeve in the tissue cavity, expanding the expandable sealing mechanism to create a seal between the expandable sealing mechanism and the inner wall of the tissue cavity, and applying a negative pressure to a region between the outer surface of the sleeve and an inner surface of the isolated portion of the tissue cavity to create a frictional force between the foam of the sleeve and the inner surface of the tissue cavity.

The method of any preceding clause, wherein the step of expanding the expandable sealing mechanism is conducted by injecting an inflation media into the expandable sealing mechanism.

The method of any preceding clause, wherein the inflation media comprises at least one of saline, mineral oil, and/or a dye-based contrast solution.

The method of any preceding clause, wherein the step of inserting the sleeve in the tissue cavity is conducted using a delivery system that includes a handle and a guide shaft.

The method of any preceding clause, further comprising a step of withdrawing the handle and the guide shaft of the delivery system from the sleeve.

The method of any preceding clause, further comprising removing the sleeve from the tissue cavity by: releasing the negative pressure, collapsing the expandable sealing mechanism, and injecting an amount of saline through a port to break the seal.

A device of any preceding clause that utilizes two redundant methods of anchoring within the bowel, wherein (i) the first method is to utilize a negative pressure based-friction anchor as described in any preceding clause, and (ii) the second method is to fixate the external portion of the device to skin with a column strength to the sleeve that is high enough to hold the anchor portion of the device in place even if there is failure of the first method of anchoring; and such that the first method of fixation is sufficient to hold the anchor portion of the device in place if there is failure of the second method.

A method of inserting the anchoring system according to any preceding clause, using an endoscope that is connected to the anchoring system.

The method of any preceding clause, wherein the anchoring system is inserted using direct visualization via the endoscope.

An anchoring system comprising a sleeve having an inner surface defining a lumen, a first expandable sealing mechanism disposed along a proximal end of the sleeve, a second expandable sealing mechanism disposed along the proximal end of the sleeve, and an air conducting rough surface material disposed on an outer surface of the sleeve, wherein (i) expansion of the first and second expandable sealing mechanisms and (ii) application of negative pressure to the anchoring system, causes a seal to form between the first and second expandable sealing mechanisms, the outer surface of the sleeve, and an inner surface of a tissue cavity.

The anchoring system of any preceding clause, further comprising a sleeve body, wherein (i) the first expandable sealing mechanism is disposed at a proximal end of the sleeve body and (ii) the second expandable sealing mechanism is disposed at a distal end of the sleeve body.

The anchoring system of any preceding clause, wherein the air conducting rough surface material is at least one of a stacked mesh matrix, a honey-comb lattice of interconnected channels, gauze, fabric, or a three-dimensional woven material.

The anchoring system of any preceding clause, wherein the distal end of the sleeve body is connected to the proximal end of the sleeve.

The anchoring system of any preceding clause, wherein the sleeve body is coextensive with the sleeve.

The anchoring system of any preceding clause, wherein the sleeve body is disposed on the proximal end of the sleeve.

The anchoring system of any preceding clause, wherein the sleeve includes a plurality of fluid lumens.

The anchoring system of any preceding clause, wherein the plurality of fluid lumens includes one or more of (i) a distal fluid lumen to provide a fluid to the first expandable sealing mechanism, (ii) a proximal fluid lumen to provide a fluid to the second expandable sealing mechanism, (iii) a flushing lumen, (iv) a contrast dye lumen, and (v) a negative pressure lumen.

The anchoring system of any preceding clause, wherein the application of negative pressure creates a frictional force that resists displacement of the sleeve.

The anchoring system of any preceding clause, wherein the application of negative pressure brings the air conducting rough surface material disposed on the outer surface of the sleeve into contact with the inner surface of the tissue cavity thereby creating the frictional force that resists displacement of the sleeve.

The anchoring system of any preceding clause, wherein the first and second expandable sealing mechanisms are expanded by providing a non-compressible fluid to expand the first and second expandable sealing mechanisms.

The anchoring system of any preceding clause, wherein the non-compressible fluid is at least one of saline, mineral oil, and a dye-based contrast solution.

The anchoring system of any preceding clause, wherein, once expanded, the first and second expandable sealing mechanisms form a substantially airtight and fluid-tight seal with the inner surface of the tissue cavity.

The anchoring system of any preceding clause, wherein the sleeve protects the inner surface of the tissue cavity from fecal flow.

The anchoring system of any preceding clause, wherein the lumen has a diameter between approximately 1 cm and approximately 6 cm.

The anchoring system of any preceding clause, herein the sleeve comprises a flexible material having a Shore A hardness between about 20 A and about 70 A.

The anchoring system of any preceding clause, wherein the air conducting rough surface material has a tensile strength of at least 50 kpa.

The anchoring system of any preceding clause, wherein the air conducting rough surface material has a thickness of between 2 mm and 150 mm.

The anchoring system of any preceding clause, wherein the first and second expandable sealing mechanisms each comprises an expandable elastomeric balloon.

The anchoring system of any preceding clause, wherein the first and second expandable sealing mechanisms each comprises multiple expandable elastomeric balloons.

The anchoring system of any preceding clause, wherein (i) the first expandable sealing mechanism comprises a single expandable elastomeric balloon, and (ii) the second expandable sealing mechanism comprises multiple expandable elastomeric balloons.

The anchoring system of any preceding clause, wherein the first and second expandable sealing mechanisms have an annular diameter that is greater than an annular diameter of the air conducting rough surface material dispersed around the sleeve.

The anchoring system of any preceding clause, wherein the sleeve has a column strength of about 3.0 lbs. to 6.0 lbs.

The anchoring system of any preceding clause, further comprising a collection bag that is disposed at a distal end of the sleeve and configured to manage enteric contents carried from the anchoring system.

The anchoring system of any preceding clause, further comprising a retention dressing to attach the anchoring system to skin.

The anchoring system of any preceding clause, further comprising an extension tubing connected to a distal end of the sleeve, the extension tubing configured to extend the anchoring system outside of a patient.

The anchoring system of any preceding clause, further comprising a scope adapter configured to connect the sleeve to at least one of an endoscope, a sigmoidoscope, or a colonoscope.

The anchoring system of any preceding clause, wherein the scope adapter comprises a main body, a compression nut, a compression sleeve, and a compression washer.

The anchoring system of any preceding clause, wherein the main body includes a plurality of threads configured to engage with a plurality of grooves provided along an interior surface of the compression nut.

The anchoring system of any preceding clause, further comprising a connector latch point configured to connect the sleeve to at least one of an endoscope, a sigmoidoscope, or a colonoscopy scope via the scope adapter.

The anchoring system of any preceding clause, further comprising a negative pressure source, wherein negative pressure is applied to the anchoring system by the negative pressure source to maintain one of a constant negative pressure or a variable negative pressure at a level between −50 mmHg and −200 mmHg.

The anchoring system of any preceding clause, further comprising a negative pressure lumen configured to provide negative pressure to the sleeve.

The anchoring system of any preceding clause, wherein the sleeve has a length that allows it to extend outside the tissue cavity.

The anchoring system of any preceding clause, wherein the lumen and the first and second expandable sealing mechanisms are compressible by normal peristaltic forces of a patient's bowel.

The anchoring system of any preceding clause, wherein the sleeve has a length that is between about 3 cm and about 25 cm.

The anchoring system of any preceding clause, wherein the sleeve and/or the sleeve body are comprised of one or more of silicone, polyurethane, thermoplastic elastomer, rubber, rubber-like material, or other polymer.

The anchoring system of any preceding clause, wherein the sleeve and the sleeve body are comprised of one continuous tubular extrusion.

The anchoring system of any preceding clause, wherein the proximal end of the sleeve is in sealed fluid communication with, and extending distal to, the distal end of the sleeve body and the second expandable sealing mechanism, wherein the proximal end of the sleeve, extending distal to the distal end of the sleeve body, is configured to cover and protect a damaged area of tissue of the tissue cavity from content flowing through the lumen of the sleeve and the sleeve body.

An anchoring system configured to be anchored within the bowel of a patient, the anchoring device comprising a sleeve configured to be positioned within the bowel of the patient, an external portion configured to extend externally from the bowel of the patient, and two redundant methods of anchoring the device within the bowel, wherein a first method of anchoring the device utilizes a negative pressure-based system that applies negative pressure to the device to create a frictional force that resists displacement of the sleeve of the device from the bowel, wherein a second method of anchoring the device fixates the external portion of the device to the skin of the patient, and wherein at least one of (i) a column strength of the sleeve is high enough to hold the device in place even if there is failure of the first method of anchoring, or (ii) the first method of anchoring is sufficient to hold the device in place even if there is failure of the second method of anchoring.

The anchoring system of any preceding clause, further comprising a third redundant method of anchoring the device, wherein the third method of anchoring utilizes first and second expandable sealing mechanisms, wherein expansion of the first and second expandable sealing mechanisms causes a seal to form between the first and second expandable sealing mechanisms and an inner surface of a tissue cavity, wherein the third method of anchoring is sufficient to hold the device in place even if there is failure of the first and/or the second method of anchoring.

The following documents are incorporated by reference herein:
Morks, A. N., Havenga, K., Ploeg, R. J., “Can intraluminal devices prevent or reduce colorectal anastomotic leakage: A review,” World J. Gastroenterol., 2011; 17(40): 4461-4469.
D'Urso, A., Komen, N., Lefevre, J. H., “Intraluminal flexible sheath for the protection of low anastomosis after anterior resection: results from a First-In-Human trial on 15 patients,” Surg. Endosc., 2019.
Kim, J. H., Kim, S., Jung, S. H., “Fecal diverting device for the substitution of defunctioning stoma: preliminary clinical study,” Surg. Endosc., 2019; 33(1): 333-340.
Reshef, A., Ben-Arie, G., Pinsk, I., “Protection of colorectal anastomosis with an intraluminal bypass device for patients undergoing an elective anterior resection: a pilot study,” Tech. in Coloproctology, 2019; 23(6): 565-571.
Kang, S. I., Kim, S. H., Jung, S. H., Kim, J. H., “The effectiveness of a fecal diverting device for prevention of septic complications in a dog model of ischemic bowel anastomosis,” Asian J. Surg., 2020; 43: 251-256 (available online Apr. 11, 2019).
Bakker, I. S., Morks, A. N., Ten Cate Hoedemaker, H. O., et al., “Randomized clinical trial of biodegradeable intraluminal sheath to prevent anastomotic leak after stapled colorectal anastomosis,” BJS Society Ltd., 2017.
   The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. An anchoring system comprising:
a sleeve having an inner surface defining a lumen;
a first expandable sealing mechanism disposed along a proximal end of the sleeve;
a second expandable sealing mechanism disposed along the proximal end of the sleeve; and
open-cell foam disposed on an outer surface of the sleeve,
wherein (i) expansion of the first and second expandable sealing mechanisms and (ii) application of negative pressure to the anchoring system, causes a seal to form between the first and second expandable sealing mechanisms, the outer surface of the sleeve, and an inner surface of a tissue cavity.

2. An anchoring system according to claim 1, further comprising a sleeve body, wherein (i) the first expandable sealing mechanism is disposed at a proximal end of the sleeve body and (ii) the second expandable sealing mechanism is disposed at a distal end of the sleeve body.

3. An anchoring system according to claim 2, wherein the distal end of the sleeve body is connected to the proximal end of the sleeve.

4. An anchoring system according to claim 2, wherein the sleeve body is coextensive with the sleeve.

5. An anchoring system according to claim 2, wherein the sleeve body is disposed on the proximal end of the sleeve.

6. An anchoring system according to claim 1, wherein the sleeve includes a plurality of fluid lumens.

7. An anchoring system according to claim 6, wherein the plurality of fluid lumens includes one or more of (i) a distal fluid lumen to provide a fluid to the first expandable sealing mechanism, (ii) a proximal fluid lumen to provide a fluid to the second expandable sealing mechanism, (iii) a flushing lumen, (iv) a contrast dye lumen, and (v) a negative pressure lumen.

8. An anchoring system according to claim 1, wherein the application of negative pressure creates a frictional force that resists displacement of the sleeve.

9. An anchoring system according to claim 8, wherein the application of negative pressure brings the open-cell foam disposed on the outer surface of the sleeve into contact with the inner surface of the tissue cavity thereby creating the frictional force that resists displacement of the sleeve.

10. An anchoring system according to claim 1, wherein the first and second expandable sealing mechanisms are expanded by providing a non-compressible fluid to expand the first and second expandable sealing mechanisms.

11. An anchoring system according to claim 10, wherein the non-compressible fluid is at least one of saline, mineral oil, and a dye-based contrast solution.

12. (canceled)

13. (canceled)

14. (canceled)

15. (canceled)

16. An anchoring system according to claim 1, wherein the open-cell foam comprises polyvinyl alcohol, polyurethane foam, or other synthetic polymer.

17. (canceled)

18. An anchoring system according to claim 1, wherein the open-cell foam has a thickness of between 2 mm and 150 mm.

19. An anchoring system according to claim 1, wherein the first and second expandable sealing mechanisms each comprises an expandable elastomeric balloon.

20. (canceled)

21. (canceled)

22. An anchoring system according to claim 1, wherein the first and second expandable sealing mechanisms have an annular diameter that is greater than an annular diameter of the open-cell foam dispersed around the sleeve.

23. An anchoring system according to claim 1, wherein the sleeve has a column strength of about 3.0 lbs. to 6.0 lbs.

24. An anchoring system according to claim 1, further comprising a collection bag that is disposed at a distal end of the sleeve and configured to manage enteric contents carried from the anchoring system.

25. (canceled)

26. (canceled)

27. An anchoring system according to claim 1, further comprising a scope adapter configured to connect the sleeve to at least one of an endoscope, a sigmoidoscope, or a colonoscope.

28. (canceled)

29. (canceled)

30. (canceled)

31. An anchoring system according to claim 1, further comprising a negative pressure source, wherein negative pressure is applied to the anchoring system by the negative pressure source to maintain one of a constant negative pressure or a variable negative pressure at a level between −50 mmHg and −200 mmHg.

32. An anchoring system according to claim 1, further comprising a negative pressure lumen configured to provide negative pressure to the sleeve.

33. An anchoring system according to claim 1, wherein the sleeve has a length that allows it to extend outside the tissue cavity.

34. An anchoring system according to claim 1, wherein the lumen and the first and second expandable sealing mechanisms are compressible by normal peristaltic forces of a patient's bowel.

35. (canceled)

36. An anchoring system according to claim 1, wherein the sleeve is comprised of one or more of silicone, polyurethane, thermoplastic elastomer, rubber, rubber-like material, or other polymer.

37. (canceled)

38. An anchoring system according to claim 2, wherein the proximal end of the sleeve is in sealed fluid communication with, and extending distal to, the distal end of the sleeve body and the second expandable sealing mechanism, wherein the proximal end of the sleeve, extending distal to the distal end of the sleeve body, is configured to cover and protect a damaged area of tissue of the tissue cavity from content flowing through the lumen of the sleeve and the sleeve body.

39-42. (canceled)

43. A method for anchoring a sleeve in a tissue cavity, the sleeve having an outer surface comprising foam for contacting an inner wall of the tissue cavity, and an expandable sealing mechanism for isolating a portion of the tissue cavity adjacent to the sleeve from a remainder of the tissue cavity, the method comprising:
inserting the sleeve in the tissue cavity;
expanding the expandable sealing mechanism to create a seal between the expandable sealing mechanism and the inner wall of the tissue cavity; and
applying a negative pressure to a region between the outer surface of the sleeve and an inner surface of the isolated portion of the tissue cavity to create a frictional force between the foam of the sleeve and the inner surface of the tissue cavity.

44. A method according to claim 43, wherein the step of expanding the expandable sealing mechanism is conducted by injecting an inflation media into the expandable sealing mechanism.

45. A method according to claim 44, wherein the inflation media comprises at least one of saline, mineral oil, and/or a dye-based contrast solution.

46. A method according to claim 43, wherein the step of inserting the sleeve in the tissue cavity is conducted using a delivery system that includes a handle and a guide shaft.

47. A method according to claim 46, further comprising a step of withdrawing the handle and the guide shaft of the delivery system from the sleeve.

48. A method according to claim 43, further comprising removing the sleeve from the tissue cavity by:
releasing the negative pressure;
collapsing the expandable sealing mechanism; and
injecting an amount of saline through a port to break the seal.

49-90. (canceled)
